(19) United States
(12) Patent Application Publication
(10) Pub. No.: US 2008/0176830 A1
(43) Pub. Date: Jul. 24, 2008

(54) Compounds, Compositions, and Methods

(76) Inventors: Nicholas D. Adams, Collegeville, PA (US); Michael Gerard Darcy, Collegeville, PA (US); Dashyant Dhanak, Collegeville, PA (US); Kevin J. Duffy, Collegeville, PA (US); Duke M. Fitch, Collegeville, PA (US); Steven David Knight, Collegeville, PA (US); Kenneth Allen Newlander, Collegeville, PA (US); Antony N. Shaw, Collegeville, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/910,731

(22) PCT Filed: Apr. 13, 2006
(86) PCT No.: PCT/US2006/014062

§ 371 Date: Oct. 4, 2007

Related U.S. Application Data
(60) Provisional application No. 60/671,299, filed on Apr. 14, 2005.

(51) Int. Cl. A61K 31/551; C07D 285/16; A61K 31/549; C07D 311/76; A61K 31/352; A61K 31/498; A61P 35/04; A61P 37/00; A61P 29/00; A61P 9/00; C07D 281/10; C07D 239/80; C07D 215/00; A61K 31/47; C07D 243/10

(52) U.S. Cl. 514/211.05; 544/11; 514/222.8; 549/399; 514/457; 546/158; 514/312; 540/500; 544/286; 514/266.31; 540/490

(57) ABSTRACT

Compounds useful for treating cellular proliferative diseases and disorders by modulating the activity of KSP are disclosed.

### FIELD OF THE INVENTION

This invention relates to compounds which are inhibitors of the mitotic kinesin KSP and are useful in the treatment of cellular proliferative diseases, for example cancer, hyperplasias, restenosis, cardiac hypertrophy, immune disorders, fungal disorders, and inflammation.

### BACKGROUND OF THE INVENTION

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426), and a fragment of the KSP gene (TRIP5) has been described (Lee, et al., Mol. Endocrinol., 9:243-54 (1995); GenBank accession number L40372). Xenopus KSP homologs (Eg5), as well as Drosophila KLP61 F/KRP1 30 have been reported.

Mitotic kinesins are attractive targets for the discovery and development of novel anti-mitotic chemotherapeutics. Accordingly, it is an object of the present invention to provide compounds, compositions and methods useful in the inhibition of KSP, a mitotic kinesin.

### SUMMARY OF THE INVENTION

In accordance with the objects outlined above, the present invention provides compounds, compositions and methods that can be used to treat diseases of proliferating cells. The compounds are KSP inhibitors, particularly human KSP inhibitors.

In one aspect, the invention relates to methods for treating cellular proliferative diseases, for treating disorders by modulating the activity of KSP, and for inhibiting KSP kinesin. The methods employ compounds represented by Formula I, II or III:

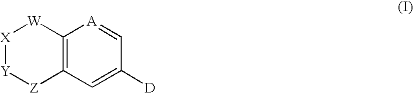

where

W is NR¹, O, CH₂, or CH(OH); where

R¹ is H, C₁₋₄alkyl, C₁₋₄alkylaryl, CO₂Bu^{t}, CO₁₋₄alkyl, CH₂CONMe₂, or CO₂CH₂Ph;

X is C═O, C═S, C═NOH, SO₂, CH₂, or CH(OH),

provided that, when W is CH(OH), X is not SO₂;

Y-Z is V—CHR²; where

### V is O, NR³, or CHR⁴;

R² is H or C₁₋₄alkyl;
R³ is H, C₁₋₂alkylOH, or C₁₋₂alkyl; and
R⁴ is H, C₁₋₄alkyl, COSEt, NH₂, OH, NHCHO, NHCOC₁₋₄alkyl, NHSO₂C₁₋₄alkyl, CO₂H,

### CH₂OH, or CONH₂;

or Y-Z is V²═CR⁵, where

### V² is N or CH; and

### R⁵ is H, Me or NH₂;

or Y-Z is V³—U, where

### V³ is CMe₂, CO or CHR⁴;

### U is NR⁷, O, S, or SO₂;

### R⁷ is H, CHO, or CH₂R⁸, and

### R³ is H, CN, CO₂Me, CONH₂, CO₂H, or CH₂OH;

or Y-Z is CH═N;

A is N or CR¹⁰, where

### R¹⁰ is H, F, CO₂H, NH₂, or NO₂ and

D is selected from:

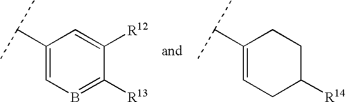

where

### B is N or CR¹¹;

### R¹¹ is H or F;

R¹² is H, halogen, Me, NH₂, NHAc, NO₂, CF₃, 1-pyrryl, or CH₂CN;
R¹³ is H, CF₃, CN, SO₂CF₃, SO₂NMe₂, SO₂C₁₋₃alkyl, SC₁₋₃alkyl, halogen, 1-indolyl, Prⁱ,

### Bu^{t}, NMe₂, or NO₂;

or R¹² and R¹³ taken together are OCF₂O;
provided that, when R¹³ is H, B and R¹² are CH and CF₃ respectively or CF and F respectively; and
R¹⁴ is CF₃ or C₂₋₅alkyl.

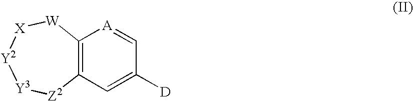

where W, X, A and D are as defined in Formula (I), and

Y² is O, NR³, CHR⁴, or CMe₂;

Y³ is CH₂, O, S, or NH;

Z² is CHR², NR⁷, O, S, or SO₂;

or Y³-Z² taken together is N═CH when Y² is CHR⁴;

provided that, when Y² is O or NR³, Y³ is CH₂ and Z² is CHR²; and
provided that, when Y³ is O, S, or NH, Z² is CHR² and Y² is CHR⁴; and
provided that, when Z² is NR⁷, O, S, or SO₂, Y² is CHR⁴, or CMe₂ and Y³ is CH₂.

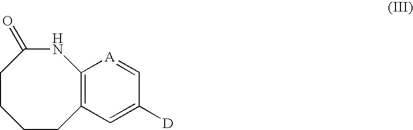

where
A and D are defined as in Formula I.

Formulas I, II and III are intended to include single stereoisomers and mixtures of stereoisomers of the compounds of these formulas.

Also included in the present invention are pharmaceutically acceptable derivatives (including salts) and solvates of compounds of Formulas I, II and III.

In one aspect, the invention relates to methods for treating cellular proliferative diseases and other disorders that can be treated by modulating KSP kinesin activity and for inhibiting KSP by the administration of a therapeutically effective amount of a compound of Formula I, II or III, or a pharmaceutically acceptable derivative or solvate thereof. Diseases and disorders that respond to therapy with compounds of the invention include cancer, hyperplasia, restenosis, cardiac hypertrophy, immune disorders, fungal disorders and inflammation.

In another aspect, the invention relates to compounds useful in inhibiting KSP kinesin. The compounds have the structures represented by Formula I, II or III, or a derivative or solvate thereof. The invention also relates to a pharmaceutical composition containing a therapeutically effective amount of a compound of Formula I, II or III, or a pharmaceutically acceptable derivative or solvate thereof admixed with at least one pharmaceutically acceptable excipient.

In an additional aspect, the present invention provides methods of screening for compounds that will bind to a KSP kinesin, for example compounds that will displace or compete with the binding of the compounds of the invention. The methods comprise combining a labeled compound of the invention, a KSP kinesin, and at least one candidate agent and determining the binding of the candidate bioactive agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate bioactive agent on the KSP kinesin activity.

### BRIEF DESCRIPTION OF THE DRAWINGS

FIG. 1 shows representative compounds of the invention.

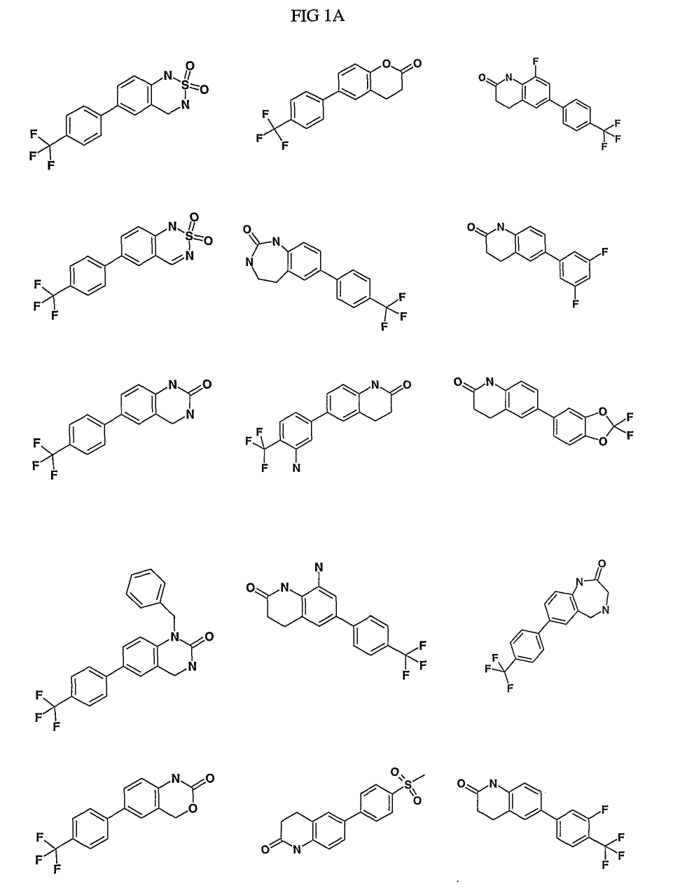

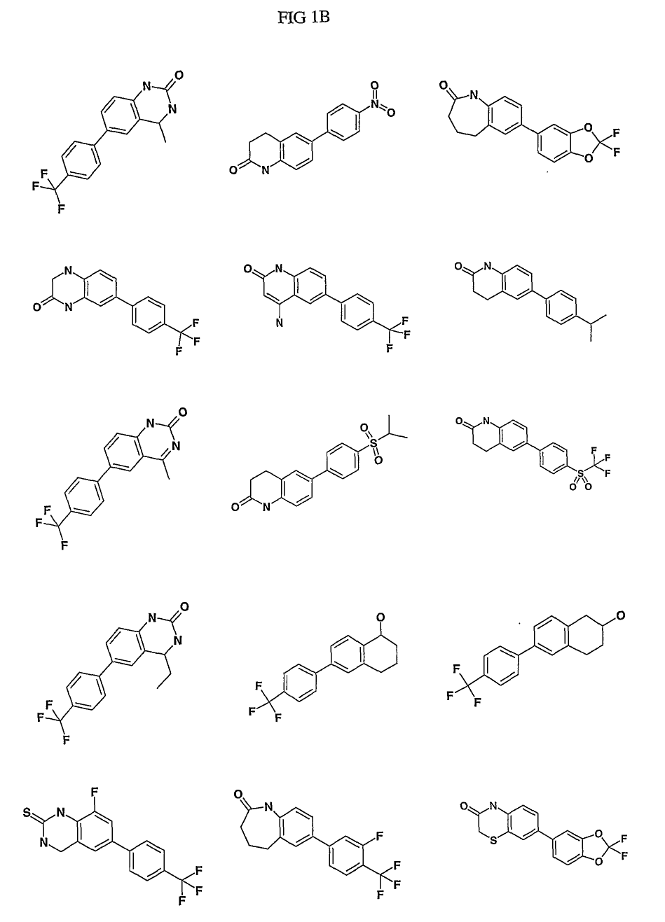

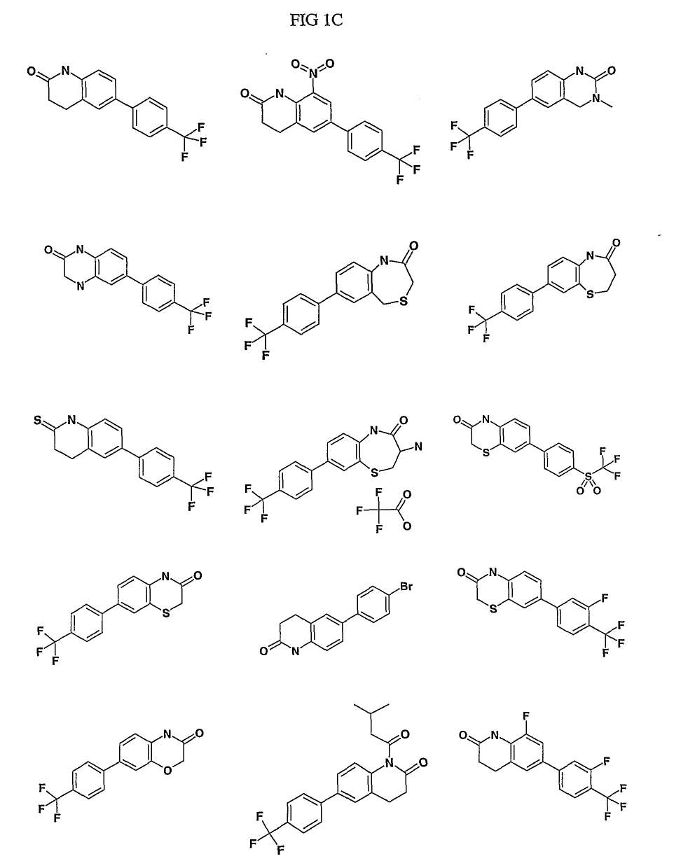

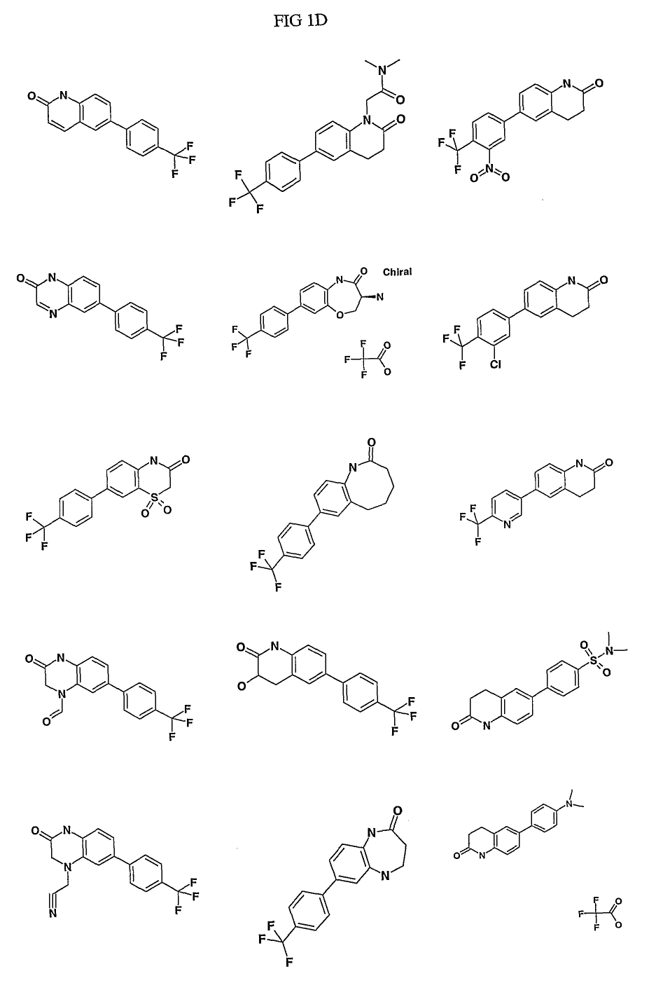

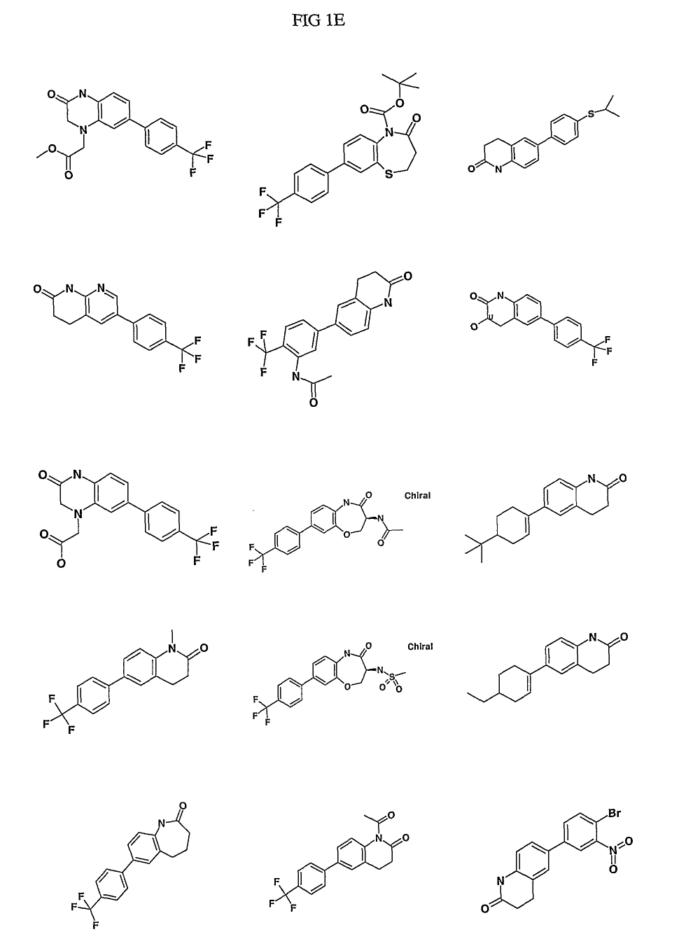

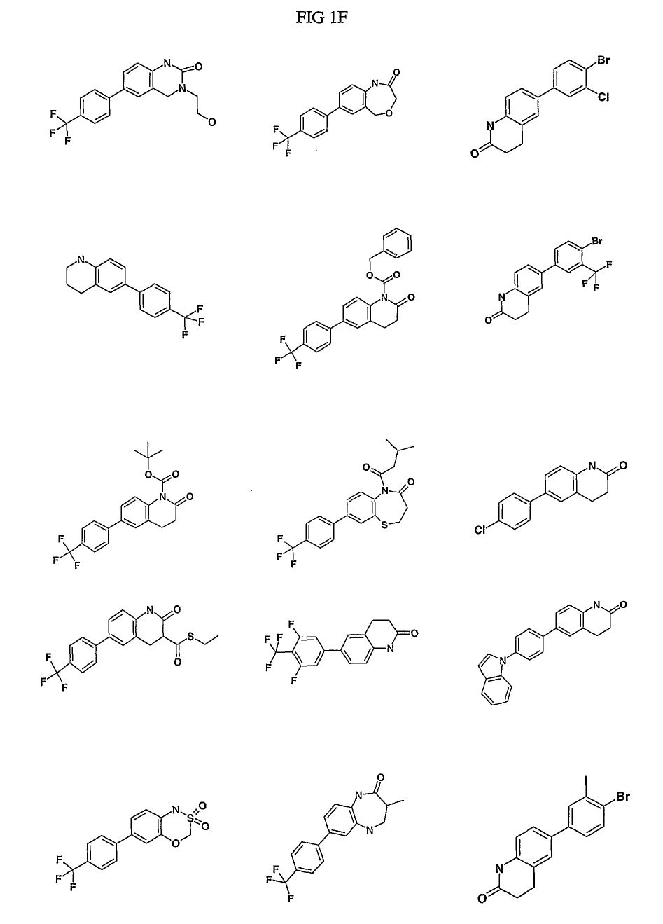

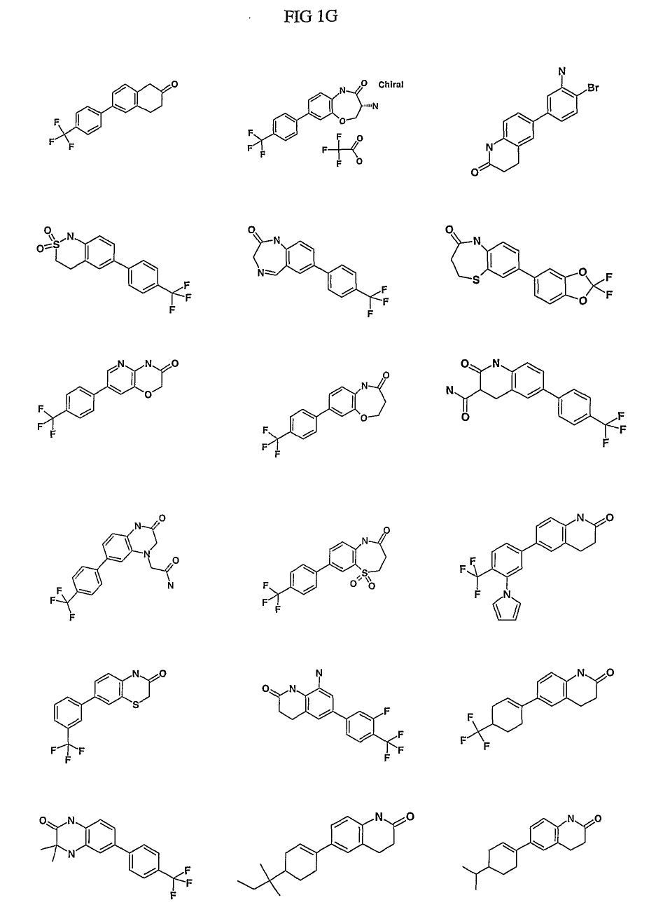

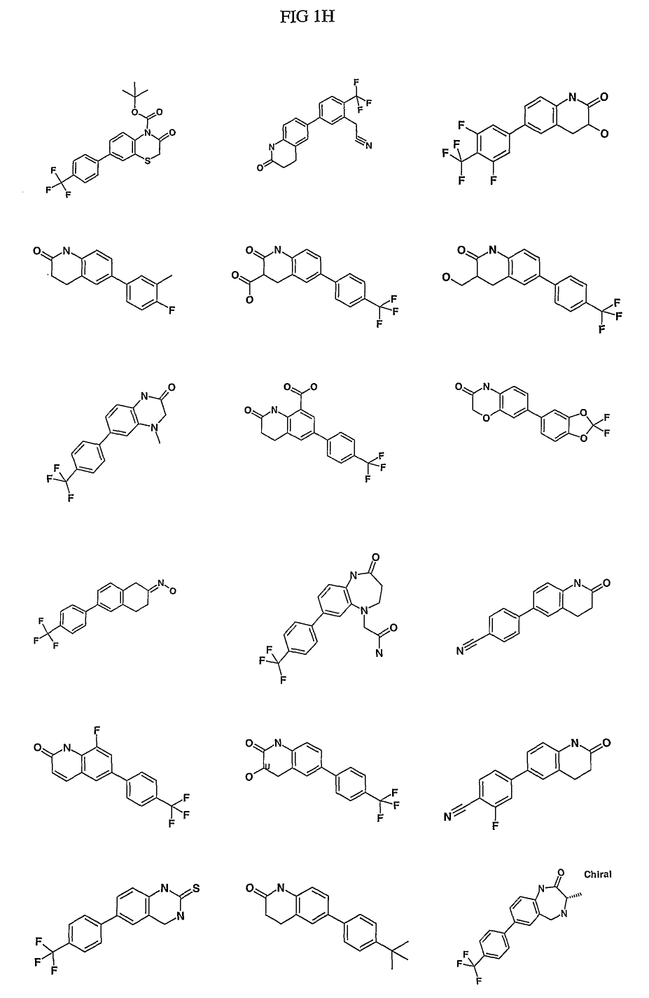

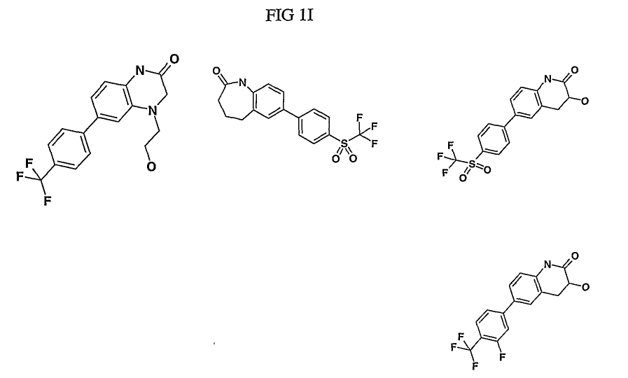

### DETAILED DESCRIPTION OF THE INVENTION

### Definitions

“Alkyl” is intended to include linear, branched, or cyclic hydrocarbon structures and combinations thereof. “Lower alkyl” refers to alkyl groups of from one to five (e.g., one to four or one to three), carbon atoms. Examples of lower alkyl groups include methyl, ethyl, propyl, isopropyl, butyl, s- and t-butyl and the like. Preferred alkyl groups are those of C₂₀ or below. More preferred alkyl groups are those of C₁₃ or below. Most preferred are alkyl groups of C₄ or below. “Cycloalkyl” is a subset of alkyl and includes cyclic hydrocarbon groups of from 3 to 13 carbon atoms. Examples of cycloalkyl groups include c-propyl, c-butyl, c-pentyl, norbornyl, adamantyl and the like. In this application, alkyl refers to alkanyl, alkenyl and alkynyl residues; it is intended to include cyclohexylmethyl, vinyl, allyl, isoprenyl and the like. “Alkylene” is another subset of alkyl, referring to the same residues as alkyl, but having two points of attachment. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl. In particular embodiments, alkyl is linear or branched hydrocarbon, more particularly lower alkyl, most particularly C₁₋₄ alkyl.

“Antimitotic” refers to a drug for inhibiting or preventing mitosis, for example, by causing metaphase arrest. Some antitumour drugs block proliferation and are considered antimitotics.

“Aryl” means a 6-membered aromatic; or a bicyclic 9- or 10-membered aromatic; or a tricyclic 12- to 14-membered aromatic. The aromatic 6- to 14-membered carbocyclic rings include, e.g., phenyl, naphthyl, indanyl, tetralinyl, and fluorenyl. In particular embodiments, aryl is phenyl or naphthyl.

“Alkylaryl” refers to a residue in which an aryl moiety is attached to the parent structure via an alkyl residue. Examples include benzyl, phenethyl, phenylvinyl, phenylallyl and the like.

“Halogen” or “halo” refers to fluorine, chlorine, bromine or iodine. Fluorine, chlorine and bromine are preferred.

It will be understood that “optional” or optionally” means that the subsequently described event or circumstance may or may not occur, and that the description includes instances where said event or circumstance occurs and instances in which it does not. It will be understood that when a group or moiety is “optionally substituted,” the group or moiety may be unsubstituted or may be substituted by one or more of the substituents defined herein, where each substituent is selected independently. It will be further understood by those skilled in the art with respect to any groups containing one or more substituents that such groups are not intended to introduce any substituent or substitution patterns that are sterically impractical and/or synthetically non-feasible and/or inherently unstable.

The alkyl and aryl groups (including alkylaryl groups) may be optionally substituted, e.g., with one or more (e.g., one to three) hydrogen atoms being replaced by substituent groups selected from lower alkyl, halo, hydroxyl (—OH), amino (NH₂), alkylamino (NHR where R is lower alkyl), or dialkylamino (NRR where each R is independently selected from lower alkyl).

“Solvate” refers to the compound formed by the interaction of a solvent and a compound of Formula I, II or III, or a pharmaceutically acceptable derivative thereof. Suitable solvates are those formed with pharmaceutically acceptable solvents, including hydrates (i.e., wherein the solvent is water). It will be understood that phrases such as “a compound of Formula I, II or III, or a pharmaceutically acceptable derivative (e.g., salt) or solvate thereof” are intended to encompass the compound of Formula I, II, or III, a pharmaceutically acceptable derivative (e.g., salt) of the compound, a solvate of the compound and a solvate of a pharmaceutically acceptable derivative (e.g., salt) of the compound.

“Pharmaceutically acceptable derivatives” of Formula I, II or III include any pharmaceutically acceptable salt, ester, or salt of such ester, of a compound of Formula I, II or III which, upon administration to the recipient is capable of providing (directly or indirectly) a compound of Formula I, II or III, or an active metabolite or residue thereof. For simplicity, in certain instances herein reference is made specifically to salts of a compound of Formula I, II or Ill. It is appreciated that other pharmaceutically acceptable derivatives, such as esters, of a compound of Formula I, II or III, are also suitable for use in the present invention in the manner specifically disclosed for salts, as though expressly set forth herein.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. In a particular embodiment ammonium, potassium, sodium, calcium, or magnesium salts are used. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Compounds described herein which contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

When desired, the R- and S-isomers may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

### Compounds of the Present Invention

The present invention is directed to a class of novel compounds that are modulators, particularly inhibitors, of mitotic kinesins. By inhibiting or modulating mitotic kinesins, but not other kinesins (e.g., transport kinesins), specific inhibition of cellular proliferation is accomplished. Thus, the present invention capitalizes on the finding that perturbation of mitotic kinesin function causes malformation or dysfunction of mitotic spindles, frequently resulting in cell cycle arrest and cell death. The methods of inhibiting a human KSP kinesin comprise contacting an inhibitor of the invention with a KSP kinesin, particularly human KSP kinesins, including fragments and variants of KSP. The inhibition can be of the ATP hydrolysis activity of the KSP kinesin and/or the mitotic spindle formation activity, such that the mitotic spindles are disrupted. Meiotic spindles may also be disrupted.

An object of the present invention is to develop inhibitors and modulators of mitotic kinesins, in particular KSP, for the treatment of disorders associated with cell proliferation. Traditionally, dramatic improvements in the treatment of cancer, one type of cell proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds, compositions and methods described herein can differ in their selectivity and are preferably used to treat diseases of proliferating cells, including, but not limited to cancer, hyperplasias, restenosis, cardiac hypertrophy, immune disorders, fungal disorders and inflammation.

Accordingly, the present invention relates to methods employing compounds represented by Formula I, II or III:

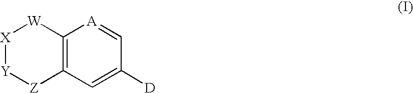

where

W is NR¹, O, CH₂, or CH(OH); where

R¹ is H, C₁₋₄alkyl, C₁₋₄alkylaryl, CO₂Bu^{t}, CO₁₋₄alkyl, CH₂CONMe₂, or CO₂CH₂Ph;

X is C═O, C═S, C═NOH, SO₂, CH₂, or CH(OH),

provided that, when W is CH(OH), X is not SO₂;

Y-Z is V—CHR²; where

### V is O, NR³, or CHR⁴;

R² is H or C₁₋₄alkyl;
R³ is H, C₁₋₂alkylOH, or C₁₋₂alkyl; and
R⁴ is H, C₁₋₄alkyl, COSEt, NH₂, OH, NHCHO, NHCOC₁₋₄alkyl, NHSO₂C₁₋₄alkyl, CO₂H, CH₂OH, or CONH₂;

or Y-Z is V²═CR⁵, where

### V² is N or CH; and

### R⁵ is H, Me or NH₂;

or Y-Z is V³—U, where

### V³ is CMe₂, CO or CHR⁴;

### U is NR⁷, O, S, or SO₂;

### R⁷ is H, CHO, or CH₂R⁸, and

### R⁸ is H, CN, CO₂Me, CONH₂, CO₂H, or CH₂OH;

or Y-Z is CH═N;

A is N or CR¹⁰, where

### R¹⁰ is H, F, CO₂H, NH₂, or NO₂ and

D is selected from:

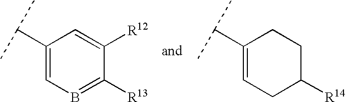

where

### B is N or CR¹¹;

### R¹¹ is H or F;

R¹² is H, halogen, Me, NH₂, NHAc, NO₂, CF₃, 1-pyrryl, or CH₂CN;
R¹³ is H, CF₃, CN, SO₂CF₃, SO₂NMe₂, SO₂C₁₋₃alkyl, SC₁₋₃alkyl, halogen, 1-indolyl, Prⁱ, Bu^{t}, NMe₂, or NO₂;
or R¹² and R¹³ taken together are OCF₂O;
provided that, when R¹³ is H, B and R¹² are CH and CF₃ respectively or CF and F respectively; and
R¹⁴ is CF₃ or C₂₋₅alkyl.

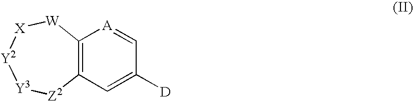

where W, X, A and D are as defined in Formula (I), and

Y² is O, NR³, CHR⁴, or CMe₂;

Y³ is CH₂, O, S, or NH;

Z² is CHR²NR⁷, O, S, or SO₂;

or Y³-Z² taken together is N═CH when Y² is CHR⁴;

provided that, when Y² is O or NR³, Y³ is CH₂ and Z² is CHR²; and
provided that, when Y³ is O, S, or NH, Z² is CHR² and Y² is CHR⁴; and
provided that, when Z² is NR⁷, O, S, or SO₂, Y² is CHR⁴, or CMe₂ and Y³ is CH₂.

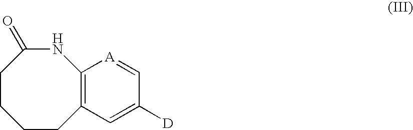

where
A and D are defined as in Formula I.

In one embodiment of Formula (I), W is NR¹. In a particular embodiment of such Formula (I), R¹ is H, i.e., a compound represented by the following formula:

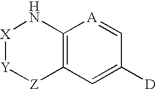

In one embodiment of such Formula (I):

### X is CO, CS, or SO₂;

Y-Z is V—CHR² wherein V is O, NR³, or CHR⁴, R³ is H, and R⁴ is H, OH, or CO₂H;

### Z is CHR² and R² is H;

### A is CR¹⁰ or N, and R¹⁰ is H or F;

### D is

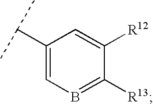

### B is CR¹¹;

R¹² is H, F, Cl, CF₃, 1-pyrryl, or NO₂; and

### R¹³ is CF₃, Cl, Br, SO₂CF₃, Prⁱ, or Bu^{t}; or

R¹² and R¹³ together are OCF₂O.

In a more particular embodiment of such Formula (I), X is C═O.

In a further particular embodiment of such Formula (I), V is CHR⁴; R⁴ is H, OH, or CO₂H; R¹² is H, F, CF₃ or NO₂; and R¹³ is CF₃ or SO₂CF₃; or R¹² and R¹³ together are OCF₂O.

In another embodiment of Formula (I):

### X is CO, CS, or SO₂;

Y-Z is V³—U wherein V³ is CMe₂, CO or CHR⁴, and R⁴ is H or CO₂H;

### U is O, S, or NR⁷ and R⁷ is H;

### A is CR¹⁰ or N, and R¹⁰ is H or F;

### D is

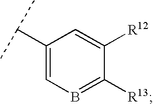

### B is CR¹¹;

R¹² is H, F, Cl, CF₃, 1-pyrryl, or NO₂; and

### R¹³ is CF₃, Cl, Br, SO₂CF₃, Prⁱ, or Bu^{t}; or

R¹² and R¹³ together are OCF₂O.

In a more particular embodiment of such Formula (I), X is C═O.

In a further particular embodiment of such Formula (I), V³ is CHR⁴; R⁴ is H or CO₂H; U is S; R¹² is H, F, CF₃ or NO₂; and R¹³ is CF₃ or SO₂CF₃; or R¹² and R¹³ together are OCF₂O.

Regarding compounds of Formula (II), in one embodiment W is NR¹. In a particular embodiment of such Formula (II), R¹ is H, i.e., a compound represented by the following formula:

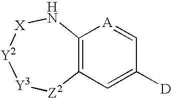

In a particular embodiment of Formula (II):

### X is CO, CS, or SO₂;

### Y² is CHR⁴ and R⁴ is H, OH or CO₂OH;

### Z² is CHR², O or S, and R² is H;

### A is CR¹⁰ or N, and R¹⁰ is H or F;

### D is

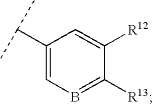

### B is CR¹¹;

R¹² is H, F, Cl, CF₃, 1-pyrryl, or NO₂; and

### R¹³ is CF₃, Cl, Br, SO₂CF₃, Prⁱ, or Bu^{t};

or R¹² and R¹³ together are OCF₂O.

In a particular embodiment of such Formula (II), X is C═O and Y² is CH₂, i.e., a compound represented by the following formula:

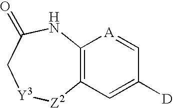

In a further particular embodiment of such Formula (II), R¹² is H, F, CF₃, or NO₂; and R¹³ is CF₃ or SO₂CF₃; or R¹² and R¹³ together are OCF₂O.

Formulas I, II and III are intended to include single stereoisomers and mixtures of stereoisomers of the compounds of these formulas.

Also included in the present invention are pharmaceutically acceptable derivatives (including salts) and solvates of compounds of Formulas I, II and III.

### Nomenclature

The compounds of Formula I, II and III can be named and numbered (e.g., using ACD/Name add-in for ISIS/Draw version 6.02) as described below.

For example, the compound of Formula I where W is NH, X is C═S, Y is NH, Z is CH₂, A is CH, and D is 4-trifluoromethyl phenyl can be named 6-[4-(trifluoromethyl)phenyl]-3,4-dihydro-2(1H)-quinazolinethione.

Similarly, the compound of Formula I where W is NH, X is C═O, Y is CH₂, Z is CH₂, A is CH, and D is 4-trifluoromethyl phenyl can be named 6-(4-Trifluoromethyl-phenyl)-3,4-dihydro-1H-quinolin-2-one.

The compound of Formula I where W is NH, X is SO₂, Y is NH, Z is CH₂, A is CH, and D is 4-trifluoromethyl phenyl can be named 6-[4-(trifluoromethyl)phenyl]-3,4-dihydro-1H-2,1,3-benzothiadiazine 2,2-dioxide.

The compound of Formula I where W is NH, X is C═O, Y is NH, Z is CH₂, A is CH and D is 4-trifluoromethyl phenyl can be named 6-[4-(trifluoromethyl)phenyl]-3,4-dihydro-2(1H)-quinazolinone.

The compound of Formula I where W is NH, X is C═O, Y is O, Z is CH₂, A is CH, and D is 4-trifluoromethyl phenyl can be named 6-[4-(trifluoromethyl)phenyl]-1,4-dihydro-2H-3,1-benzoxazin-2-one.

The compound of Formula II where W is NH, X is C═O, Y², Y³ and Z² are each CH₂, A is CH, and D is 3-fluoro, 4-trifluoromethyl phenyl can be named 7-(fluoro-trifluoromethyl-phenyl)-1,2,4,5-tetrahydro-benzo[b]azepin-2-one.

The compound of Formula II where W is NH, X is C═O, Y² and Z² are each CH₂, Y³ is S, A is CH, and D is 4-trifluoromethyl phenyl can be named 7-[4-(trifluoromethyl)phenyl]-1,5-dihydro-4,1-benzothiazepin-2(3H)-one.

The compound of Formula III where A is CH and D is 4-trifluoromethyl phenyl can be named 8-[4-(trifluoromethyl)phenyl]-3,4,5,6-tetrahydro-1-benzazocin-2(1H)one.

Particular examples of compounds of the present invention include:
6-[4-(trifluoromethyl)phenyl]-3,4-dihydro-1H-2,1,3-benzothiadiazine 2,2-dioxide
6-[4-(trifluoromethyl)phenyl]-1H-2,1,3-benzothiadiazine 2,2-dioxide
6-[4-(trifluoromethyl)phenyl]-3,4-dihydro-2(1H)-quinazolinone
1-(phenylmethyl)-6-[4-(trifluoromethyl)phenyl]-3,4-dihydro-2(1H)-quinazolinone
6-[4-(trifluoromethyl)phenyl]-3,4-dihydro-2(1H)-quinazolinethione
6-[4-(trifluoromethyl)phenyl]-1,4-dihydro-2H-3,1-benzoxazin-2-one
4-methyl-6-[4-(trifluoromethyl)phenyl]-3,4-dihydro-2(1H)-quinazolinone
7-[4-(Trifluoromethyl)phenyl]-3,4-dihydro-2(1H)-quinoxalinone
4-methyl-6-[4-(trifluoromethyl)phenyl]-2(1H)-quinazolinone
4-ethyl-6-[4-(trifluoromethyl)phenyl]-3,4-dihydro-2(1H)-quinazolinone
8-fluoro-6-[4-(trifluoromethyl)phenyl]-3,4-dihydro-2(1H)-quinazolinethione
6-(4-Trifluoromethyl-phenyl)-3,4-dihydro-1H-quinolin-2-one
6-[4-(Trifluoromethyl)phenyl]-3,4-dihydro-2(1H)-quinoxalinone
6-[4-(Trifluoromethyl)phenyl]-3,4-dihydro-2(1H)-quinolinethione
7-[4-(trifluoromethyl)phenyl]-2H-1,4-benzothiazin-3(4H)-one
7-[4-(trifluoromethyl)phenyl]-2H-1,4-benzoxazin-3(4H)-one
6-[4-(Trifluoromethyl)phenyl]-2(1H)-quinolinone
6-[4-(Trifluoromethyl)phenyl]-2(1H)-quinoxalinone
7-[4-(trifluoromethyl)phenyl]-2H-1,4-benzothiazin-3(4H)-one 1,1-dioxide
3-Oxo-7-[4-(trifluoromethyl)phenyl]-3,4-dihydro-1(2H)-quinoxalinecarbaldehyde
[3-Oxo-7-[4-(trifluoromethyl)phenyl]-3,4-dihydro-1(2H)-quinoxalinyl]acetonitrile
Methyl [3-oxo-7-[4-(trifluoromethyl)phenyl]-3,4-dihydro-1(2H)-quinoxalinyl]acetate
6-[4-(trifluoromethyl)phenyl]-3,4-dihydro-1,8-naphthyridin-2(1H)-one
[3-Oxo-7-[4-(trifluoromethyl)phenyl]-3,4-dihydro-1(2H)-quinoxalinyl]acetic acid
1-methyl-6-[4-(trifluoromethyl)phenyl]-3,4-dihydro-2(1H)-quinolinone
7-[4-(trifluoromethyl)phenyl]-1,3,4,5-tetrahydro-2H-1-benzazepin-2-one
3-(2-hydroxyethyl)-6-[4-(trifluoromethyl)phenyl]-3,4-dihydro-2(1H)-quinazolinone
6-[4-(Trifluoromethyl)phenyl]-1,2,3,4-tetrahydroquinoline
1,1-dimethylethyl 2-oxo-6-[4-(trifluoromethyl)phenyl]-3,4-dihydro-1(2H)-quinolinecarboxylate
S-ethyl 2-oxo-6-[4-(trifluoromethyl)phenyl]-1,2,3,4-tetrahydro-3-quinolinecarbothioate
6-[4-(trifluoromethyl)phenyl]-1H-4,2,1-benzoxathiazine 2,2-dioxide
6-[4-(trifluoromethyl)phenyl]-3,4-dihydro-2(1H)-naphthalenone
6-[4-(Trifluoromethyl)phenyl]-3,4-dihydro-1H-2,1-benzothiazine 2,2-dioxide
7-[4-(trifluoromethyl)phenyl]-2H-pyrido[3,2-b][1,4]oxazin-3(4H)-one
2-[3-oxo-7-[4-(trifluoromethyl)phenyl]-3,4-dihydro-1(2H)-quinoxalinyl]acetamide
7-[3-(trifluoromethyl)phenyl]-2H-1,4-benzothiazin-3(4H)-one
3,3-Dimethyl-6-[4-(trifluoromethyl)phenyl]-3,4-dihydro-2(1H)-quinoxalinone
1,1-dimethylethyl 3-oxo-7-[4-(trifluoromethyl)phenyl]-2,3-dihydro-4H-1,4-benzothiazine-4-carboxylate
6-(4-Fluoro-3-methyl-phenyl)-3,4-dihydro-1H-quinolin-2-one
4-(2-hydroxyethyl)-6-[4-(trifluoromethyl)phenyl]-3,4-dihydro-2(1H)-quinoxalinone
4-methyl-6-[4-(trifluoromethyl)phenyl]-3,4-dihydro-2(1H)-quinoxalinone
6-[4-(trifluoromethyl)phenyl]-3,4-dihydro-2(1H)-naphthalenone oxime
8-fluoro-6-[4-(trifluoromethyl)phenyl]-2(1H)-quinolinone
8-fluoro-6-[4-(trifluoromethyl)phenyl]-3,4-dihydro-2(1H)-quinolinone
6-(3,5-Difluoro-phenyl)-3,4-dihydro-1H-quinolin-2-one
6-(2,2-Difluoro-benzo[1,3]dioxol-5-yl)-3,4-dihydro-1H-quinolin-2-one
7-[4-(trifluoromethyl)phenyl]-1,3,4,5-tetrahydro-2H-1,4-benzodiazepin-2-one
6-(Fluoro-trifluoromethyl-phenyl)-3,4-dihydro-1H-quinolin-2-one
7-(2,2-Difluoro-benzo[1,3]dioxol-5-yl)-1,3,4,5-tetrahydro-benzo[b]azepin-2-one
6-(4-Isopropyl-phenyl)-3,4-dihydro-1H-quinolin-2-one
6-(4-Trifluoromethanesulfonyl-phenyl)-3,4-dihydro-1H-quinolin-2-one
±6-[4-(trifluoromethyl)phenyl]-1,2,3,4-tetrahydro-2-naphthalenol
7-(2,2-Difluorobenzo[1,3]dioxol-5-yl)-4H-benzo[1,4]thiazin-3-one
3-methyl-6-[4-(trifluoromethyl)phenyl]-3,4-dihydro-2(1H)-quinazolinone
8-[4-(trifluoromethyl)phenyl]-2,3-dihydro-1,5-benzothiazepin-4(5H)-one
7-(4-Trifluoromethanesulfonyl-phenyl)-4H-benzo[1,4]thiazin-3-one
7-(Fluoro-trifluoromethyl-phenyl)-4H-benzo[1,4]thiazin-3-one
8-fluoro-6-[3-fluoro-4-(trifluoromethyl)phenyl]-3,4-dihydro-2(1H)-quinolinone
6-[3-nitro-4-(trifluoromethyl)phenyl]-3,4-dihydro-2(1H)-quinolinone
6-[3-chloro-4-(trifluoromethyl)phenyl]-3,4-dihydro-2(1H)-quinolinone
6-[6-(trifluoromethyl)-3-pyridinyl]-3,4-dihydro-2(1H)-quinolinone
N,N-dimethyl-4-(2-oxo-1,2,3,4-tetrahydro-6-quinolinyl)benzenesulfonamide
6-[4-(dimethylamino)phenyl]-3,4-dihydro-2(1H)-quinolinone
6-(4-tert-Butyl-phenyl)-3,4-dihydro-1H-quinolin-2-one
6-{4-[(1-methylethyl)thio]phenyl}-3,4-dihydro-2(1H)-quinolinone
6-[4-(trifluoromethyl)phenyl]-3,4-dihydro-2H-chromen-2-one
7-[4-(trifluoromethyl)phenyl]-1,3,4,5-tetrahydro-2H-1,3-benzodiazepin-2-one
6-[3-amino-4-(trifluoromethyl)phenyl]-3,4-dihydro-2(1H)-quinolinone
8-amino-6-[4-(trifluoromethyl)phenyl]-3,4-dihydro-2(1H)-quinolinone
7-(4-Trifluoromethanesulfonyl-phenyl)-1,3,4,5-tetrahydro-benzo[b]azepin-2-one
6-[4-(methylsulfonyl)phenyl]-3,4-dihydro-2(1H)-quinolinone
6-(4-nitrophenyl)-3,4-dihydro-2(1H)-quinolinone
4-Amino-6-[4-(trifluoromethyl)phenyl]-2(1H)-quinolinone
6-{4-[(1-methylethyl)sulfonyl]phenyl}-3,4-dihydro-2(1H)-quinolinone
6-[4-(trifluoromethyl)phenyl]-1,2,3,4-tetrahydro-1-naphthalenol
7-(Fluoro-trifluoromethyl-phenyl)-1,3,4,5-tetrahydro-benzo[b]azepin-2-one
8-nitro-6-[4-(trifluoromethyl)phenyl]-3,4-dihydro-2(1H)-quinolinone
7-[4-(trifluoromethyl)phenyl]-1,5-dihydro-4,1-benzothiazepin-2(3H)-one
3-amino-8-[4-(trifluoromethyl)phenyl]-2,3-dihydro-1,5-benzothiazepin-4(5H)-one trifluoroacetate
6-(4-bromophenyl)-3,4-dihydro-2(1H)-quinolinone
1-(3-methylbutanoyl)-6-[4-(trifluoromethyl)phenyl]-3,4-dihydro-2(1H)-quinolinone
N,N-dimethyl-2-[2-oxo-6-[4-(trifluoromethyl)phenyl]-3,4-dihydro-1(2H)-quinolinyl]acetamide
(3S)-3-amino-8-[4-(trifluoromethyl)phenyl]-2,3-dihydro-1,5-benzoxazepin-4(5H)-one trifluoroacetate
8-[4-(trifluoromethyl)phenyl]-3,4,5,6-tetrahydro-1-benzazocin-2(1H)-one
±3-Hydroxy-6-[4-(trifluoromethyl)phenyl]-3,4-dihydro-2(1H)-quinolinone
7-[4-(trifluoromethyl)phenyl]-1,3,4,5-tetrahydro-2H-1,5-benzodiazepin-2-one
1,1-dimethylethyl 4-oxo-8-[4-(trifluoromethyl)phenyl]-3,4-dihydro-1,5-benzothiazepine-5(2H)-carboxylate
N-[5-(2-oxo-1,2,3,4-tetrahydro-6-quinolinyl)-2-(trifluoromethyl)phenyl]acetamide
N-{(3S)-4-oxo-8-[4-(trifluoromethyl)phenyl]-2,3,4,5-tetrahydro-1,5-benzoxazepin-3-yl}acetamide
N-{(3S)-4-oxo-8-[4-(trifluoromethyl)phenyl]-2,3,4,5-tetrahydro-1,5-benzoxazepin-3-yl}methanesulfonamide
1-acetyl-6-[4-(trifluoromethyl)phenyl]-3,4-dihydro-2(1H)-quinolinone
7-[4-(trifluoromethyl)phenyl]-1,5-dihydro-4,1-benzoxazepin-2(3H)-one
phenylmethyl 2-oxo-6-[4-(trifluoromethyl)phenyl]-3,4-dihydro-1(2H)-quinolinecarboxylate
5-(3-methylbutanoyl)-8-[4-(trifluoromethyl)phenyl]-2,3-dihydro-1,5-benzothiazepin-4(5H)-one
6-[3,5-difluoro-4-(trifluoromethyl)phenyl]-3,4-dihydro-2(1H)-quinolinone
3-methyl-7-[4-(trifluoromethyl)phenyl]-1,3,4,5-tetrahydro-2H-1,5-benzodiazepin-2-one
(3R)-3-amino-8-[4-(trifluoromethyl)phenyl]-2,3-dihydro-1,5-benzoxazepin-4(5H)-one trifluoroacetate
7-[4-(Trifluoromethyl)phenyl]-1,3-dihydro-2H-1,4-benzodiazepin-2-one
8-[4-(trifluoromethyl)phenyl]-2,3-dihydro-1,5-benzoxazepin-4(5H)-one
8-[4-(trifluoromethyl)phenyl]-2,3-dihydro-1,5-benzothiazepin-4(5H)-one 1,1-dioxide
8-amino-6-[3-fluoro-4-(trifluoromethyl)phenyl]-3,4-dihydro-2(1H)-quinolinone
6-[4-(1,1-dimethylpropyl)-1-cyclohexen-1-yl]-3,4-dihydro-2(1H)-quinolinone
[5-(2-oxo-1,2,3,4-tetrahydro-6-quinolinyl)-2-(trifluoromethyl)phenyl]acetonitrile
2-Oxo-6-[4-(trifluoromethyl)phenyl]-1,2,3,4-tetrahydro-3-quinolinecarboxylic acid
(3S)-3-methyl-7-[4-(trifluoromethyl)phenyl]-1,3,4,5-tetrahydro-2H-1,4-benzodiazepin-2-one
2-Oxo-6-[4-(trifluoromethyl)phenyl]-1,2,3,4-tetrahydro-8-quinolinecarboxylic acid
2-{4-oxo-8-[4-(trifluoromethyl)phenyl]-2,3,4,5-tetrahydro-1H-1,5-benzodiazepin-1-yl}acetamide
3-Hydroxy-6-[4-(trifluoromethyl)phenyl]-3,4-dihydro-2(1H)-quinolinone
3-Hydroxy-6-[4-(trifluoromethyl)phenyl]-3,4-dihydro-2(1H)-quinolinone
6-[4-(1,1-dimethylethyl)-1-cyclohexen-1-yl]-3,4-dihydro-2(1H)-quinolinone
6-(4-ethyl-1-cyclohexen-1-yl)-3,4-dihydro-2(1H)-quinolinone
6-(4-bromo-3-nitrophenyl)-3,4-dihydro-2(1H)-quinolinone
6-(4-bromo-3-chlorophenyl)-3,4-dihydro-2(1H)-quinolinone
6-[4-bromo-3-(trifluoromethyl)phenyl]-3,4-dihydro-2(1H)-quinolinone
6-(4-chlorophenyl)-3,4-dihydro-2(1H)-quinolinone
6-[4-(1H-indol-1-yl)phenyl]-3,4-dihydro-2(1H)-quinolinone
6-(4-bromo-3-methylphenyl)-3,4-dihydro-2(1H)-quinolinone
6-(3-amino-4-bromophenyl)-3,4-dihydro-2(1H)-quinolinone
8-(2,2-Difluoro-benzo[1,3]dioxol-5-yl)-2,3-dihydro-5H-benzo[b][1,4]thiazepin-4-one
2-Oxo-6-[4-(trifluoromethyl)phenyl]-1,2,3,4-tetrahydro-3-quinolinecarboxamide
6-[3-(1H-pyrrol-1-yl)-4-(trifluoromethyl)phenyl]-3,4-dihydro-2(1H)-quinolinone
6-[4-(trifluoromethyl)-1-cyclohexen-1-yl]-3,4-dihydro-2(1H)-quinolinone
6-[4-(1-methylethyl)-1-cyclohexen-1-yl]-3,4-dihydro-2(1H)-quinolinone
±6-[3,5-difluoro-4-(trifluoromethyl)phenyl]-3-hydroxy-3,4-dihydro-2(1H)-quinolinone
±3-(Hydroxymethyl)-6-[4-(trifluoromethyl)phenyl]-3,4-dihydro-2(1H)-quinolinone
7-(2,2-Difluoro-benzo[1,3]dioxol-5-yl)-4H-benzo[1,4]oxazin-3-one
4-(2-oxo-1,2,3,4-tetrahydro-6-quinolinyl)benzonitrile
2-fluoro-4-(2-oxo-1,2,3,4-tetrahydro-6-quinolinyl)benzonitrile
±3-hydroxy-6-{4-[(trifluoromethyl)sulfonyl]phenyl}-3,4-dihydro-2(1H)-quinolinone
±6-[3-fluoro-4-(trifluoromethyl)phenyl]-3-hydroxy-3,4-dihydro-2(1H)-quinolinone;
   and pharmaceutically acceptable derivatives and solvates thereof.

The compounds of the invention may be prepared as shown in the General Methods and as described below, utilizing techniques well known in the art. The starting materials for the schemes shown in the figures are commercially available, e.g., from Aldrich Chemical Company, Milwaukee, Wis. or may be readily prepared by those skilled in the art using commonly employed synthetic methodology.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Once made, the compounds of the invention find use in a variety of applications. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In a preferred embodiment, the compounds of the invention are used to modulate mitotic spindle formation, thus causing prolonged cell cycle arrest in mitosis. By “modulate” herein is meant altering mitotic spindle formation, including increasing and decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to and/or modulate the activity of a mitotic kinesin, KSP. In a preferred embodiment, the KSP is human KSP, although KSP kinesins from other organisms may also be used. In this context, modulate means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. Nos. 6,414,121 and 6,437,115, hereby incorporated by reference in their entirety. In addition, other mitotic kinesins may be used in the present invention. However, the compounds of the invention have been shown to have specificity for KSP.

For assay of activity, generally either KSP or a compound according to the invention is non-diffusably bound to an insoluble support having isolated sample receiving areas (e.g., a microtiter plate, an array, etc.). The insoluble support may be made of any composition to which the compounds can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the compound is not crucial so long as it is compatible with the reagents and overall methods of the invention, maintains the activity of the compound and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

The anti-mitotic agents of the invention may be used on their own to modulate the activity of a mitotic kinesin, particularly KSP. In this embodiment, the anti-mitotic agents of the invention are combined with KSP and the activity of KSP is assayed. Kinesin activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, Anal. Biochem. 242 (1):20-5; Gittes et al., 1996, Biophys. J. 70(1): 418-29; Shirakawa et al., 1995, J. Exp. Biol. 198:1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Preferably, solution based assays are utilized. U.S. patent application Ser. No. 09/314,464, filed May 18, 1999 (U.S. Pat. No. 6,410,254), hereby incorporated by reference in its entirety, describes such assays. Alternatively, conventional methods are used. For example, Pᵢ release from kinesin can be quantified. In one preferred embodiment, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules.

Different types of modulating agents can be detected in the above assays. In one embodiment, the effect of a modulating agent is independent of the concentration of microtubules and ATP. In another embodiment, the effect of the agents on kinesin ATPase can be decreased by increasing the concentrations of ATP, microtubules or both (i.e., the effect can be increased by decreasing the concentrations of ATP, microtubules or both). In yet another embodiment, the effect of the modulating agent is increased by increasing concentrations of ATP, microtubules or both.

Agents that modulate the biochemical activity of KSP in vitro may then be screened in vivo. Methods for such agents in vivo include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability. See for example, U.S. patent application “Methods of Screening for Modulators of Cell Proliferation and Methods of Diagnosing Cell Proliferation States,” filed Oct. 22, 1999, Ser. No. 09/428,156 (U.S. Pat. No. 6,617,115), hereby incorporated by reference in its entirety.

In addition to the assays described above, microscopic methods for monitoring spindle formation and malformation are well known to those of skill in the art (see, e.g., Whitehead and Rattner (1998), J. Cell Sci. 111:2551-61; Galgio et al, (1996) J. Cell biol., 135:399-414).

The compounds of the invention inhibit the KSP kinesin. One measure of inhibition is IC₅₀, defined as the concentration of the compound at which the activity of KSP is decreased by fifty percent relative to a control. Preferred compounds have IC₅₀'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and more preferably less than about 10 nM. Measurement of IC₅₀ is done using an ATPase assay.

Another measure of inhibition is Kᵢ. For compounds with IC₅₀'s less than 1 μM, the Kᵢ or K_{d} is defined as the dissociation rate constant for the interaction of the compounds described herein with KSP. Preferred compounds have Kᵢ's of less than about 100 μM, with preferred embodiments having Kᵢ's of less than about 10 μM, with particularly preferred embodiments having Kᵢ's of less than about 1 μM, and especially preferred embodiments having Kᵢ's of less than about 100 nM. The K for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Preferred compounds have GI₅₀'s of less than about 1 mM. The level of preferability of embodiments is a function of their GI₅₀: those having GI₅₀'s of less than about 20 μM are more preferred; those having GI₅₀'s of 10 μM more so; those having GI₅₀ of less than about 1 μM more so; those having GI₅₀'s of less than about 100 nM even more so. Measurement of GI₅₀ is done using a cell proliferation assay.

The compounds, compositions and methods of the invention are used to treat cellular proliferation diseases. Disease states which can be treated by the compounds, compositions and methods provided herein include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. Thus, in one embodiment, the invention herein includes application to cells or individuals afflicted or impending affliction with any one of these disorders or states.

The compounds, compositions and methods provided herein are particularly deemed useful for the treatment of cancer including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Accordingly, the compounds of the invention are administered to cells. By “administered” herein is meant administration of a therapeutically effective dose of the anti-mitotic agents of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

Anti-mitotic agents having the desired pharmacological activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-99.9 wt. %. The compounds of Formula I, II and III, and the pharmaceutically acceptable derivatives and solvates thereof can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of an active agent of the present invention.

In a preferred embodiment, the pharmaceutical compositions are in a water soluble form, such as pharmaceutically acceptable salts, which is meant to include both acid and base addition salts.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Pharmaceutical formulations include a compound of Formula I, II or III, or a pharmaceutically acceptable derivative or solvate thereof, and one or more pharmaceutically acceptable excipients. As is known in the art, pharmaceutical excipients are secondary ingredients that function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995). The concentration of a therapeutically active agent in a formulation can vary widely, from about 0.1 to 99.9 wt. %, depending on the nature of the formulation.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colorants, flavorants, sweetening agents, polymers, waxes or other solubility-modulating materials.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., 1/6 M; and
sterile water for injection
   The pH of such IV fluids may vary, and will typically be from 3.5 to 8 as known in the art.

The administration of the anti-mitotic agents of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the anti-mitotic agents may be directly applied as a solution or spray.

To employ the compounds of the invention in a method of screening for compounds that bind to KSP kinesin, the KSP is bound to a support, and a compound of the invention (which is an anti-mitotic agent) is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the anti-mitotic agent to KSP may be done in a number of ways. In a preferred embodiment, the anti-mitotic agent (the compound of the invention) is labeled, for example, with a fluorescent or radioactive moiety and binding determined directly. For example, this may be done by attaching all or a portion of KSP to a solid support, adding a labeled anti-mitotic agent (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the anti-mitotic agents.

The compounds of the invention may also be used as competitors to screen for additional drug candidates. “Candidate bioactive agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, preferred embodiments exclude molecules already known to bind to that particular protein, for example, polymer structures such as microtubules, and energy sources such as ATP. Preferred embodiments of assays herein include candidate agents which do not bind the cellular proliferation protein in its endogenous native state termed herein as “exogenous” agents. In another preferred embodiment, exogenous agents further exclude antibodies to KSP.

Candidate agents can encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding and lipophilic binding, and typically include at least an amine, carbonyl, hydroxyl, ether, or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Competitive screening assays may be done by combining KSP and a drug candidate in a first sample. A second sample comprises a anti-mitotic agent, KSP and a drug candidate. This may be performed in either the presence or absence of microtubules. The binding of the drug candidate is determined for both samples, and a change, or difference in binding between the two samples indicates the presence of an agent capable of binding to KSP and potentially modulating its activity. That is, if the binding of the drug candidate is different in the second sample relative to the first sample, the drug candidate is capable of binding to KSP.

In a preferred embodiment, the binding of the candidate agent is determined through the use of competitive binding assays. In this embodiment, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In one embodiment, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate agent. Displacement of the competitor is an indication the candidate agent is binding to KSP and thus is capable of binding to, and potentially modulating, the activity of KSP. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

It may be of value to identify the binding site of KSP. This can be done in a variety of ways. In one embodiment, once KSP has been identified as binding to the anti-mitotic agent, KSP is fragmented or modified and the assays repeated to identify the necessary components for binding.

Modulation is tested by screening for candidate agents capable of modulating the activity of KSP comprising the steps of combining a candidate agent with KSP, as above, and determining an alteration in the biological activity of KSP. Thus, in this embodiment, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native KSP, but cannot bind to modified KSP.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### General Methods

The compounds of this invention may be prepared according to the general process outlined in Schemes 1-25 and described in Examples 1-130 below.

The following abbreviations and terms have the indicated meanings throughout: Ac represents acetyl, Ar represents aryl, BNB represents 4-bromomethyl-3-nitrobenzoic acid, Boc represents t-butyloxy carbonyl, br represents broad, Bu represents butyl, c- represents cyclo, CBZ represents carbobenzoxy represents benzyloxycarbonyl, d represents doublet, DBU represents diazabicyclo[5.4.0]undec-7-ene, DCM represents dichloromethane represents methylene chloride represents CH₂Cl₂, DCE represents dichloroethylene, DEAD represents diethyl azodicarboxylate, DIBAL represents di-isobutylaluminium hydride, DIC represents diisopropylcarbodiimide, DIEA represents N,N-diisopropylethyl amine, DMAP represents 4-N,N-dimethylaminopyridine, DMF represents N,N-dimethylformamide, DMSO represents dimethyl sulfoxide, dppf)PdCl₂.CH₂Cl₂ represents [1,1′-bis(diphenylphosphino)ferrocene]dichloropalladium(II)dichloromethane adduct, DVB represents 1,4-divinylbenzene, EDC represents 1-[3-(dimethylamino)propyl]-3-ethylcarbodiimide hydrochloride, EEDQ represents 2-ethoxy-1-ethoxycarbonyl-1,2-dihydroquinoline, ESMS represents electrospray mass spectrometry, Et represents ethyl, EtOAc represents ethyl acetate, Fmoc represents 9-fluorenylmethoxycarbonyl, GC represents gas chromatography, HATU represents O-(7-Azabenzotriazol-1-yl)-1,1,3,3-tetramethyluronium, hexafluorophosphate, HMDS represents hexamethyldisilazane, HOAc or AcOH represents acetic acid, HOBt represents hydroxybenzotriazole, HPLC represents high pressure liquid chromatography, Lawesson's reagent represents 2,4-bis(4-methoxyphenyl)-1,3-dithia-2,4-diphosphetane-2,4-disulfide, LDA represents lithium di-isopropylamide, m represents multiplet, mCPBA or MCPBA represents 3-chloroperoxybenzoic acid, Me represents methyl, Ms represents methanesulfonyl, MHz represents megahertz, MS (ES) or ESMS represents electrospray mass spectrometry, MTBE represents methyl t-butyl ether, NBS represents N-bromosuccinimide, NMO represents N-methylmorpholine oxide, NMP represents N-methylpyrrolidinone, NMR represents nuclear magnetic resonance, o-Tol represents 2-methylphenyl, Pd/C represents palladium-on-charcoal, PEG represents polyethylene glycol, Ph represents phenyl, PhOH represents phenol, PfP represents pentafluorophenol, PPTS represents pyridinium p-toluenesulfonate, Py represents pyridine, PyBroP represents bromo-tris-pyrrolidino-phosphonium hexafluorophosphate, rt or RT represent room temperature, sat'd represents saturated, s represents singlet, s- represents secondary, t represents triplet, t- represents tertiary, TBDMS represents t-butyldimethylsilyl, TES represents triethylsilyl, TFA represents trifluoroacetic acid, THF represents tetrahydrofuran, TMOF represents trimethyl orthoformate, TMS represents trimethylsilyl, tosyl or Ts represents p-toluenesulfonyl, and Trt represents triphenylmethyl.

Compounds of formula (I), (II) and (III) can be readily prepared according to the following schemes.

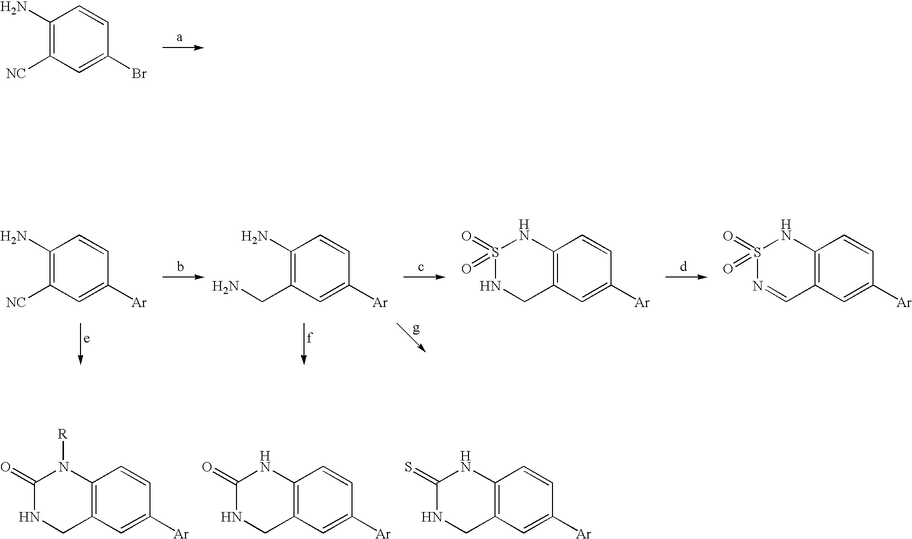

a) ArB(OH)₂, Pd catalyst, aq base, solvent, heat; b) LiAlH₄, THF; c) H₂NSO₂NH₂, pyridine, heat; d) KMnO₄, base; e) 1. NaH, RBr, THF, 2. LiAlH₄, THF, 3. Triphosgene, THF; f) Triphosgene, THF; g) EtOCS₂K, pyridine, heat

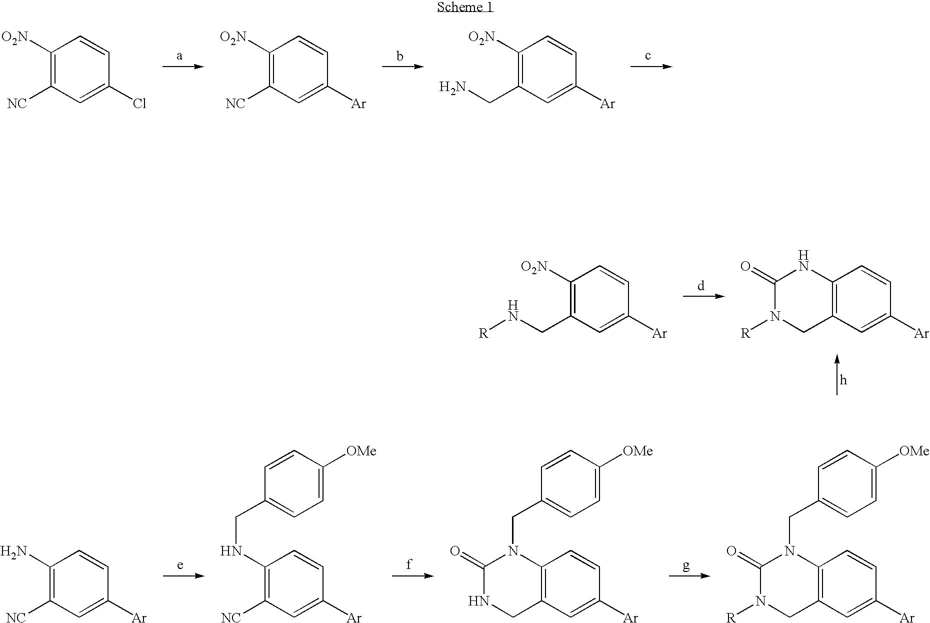

a) ArB(OH)₂, Pd catalyst, aq base, solvent, heat; b) BH₃.THF, THF; c) RCHO, NaHB(OAc)₃, EtOH; d) 1. H₂, Pd/C, MeOH, 2. Triphosgene, THF; e) 4-MeOPhCH₂Cl, Et₃N, MeCN, heat; f) 1. LiAlH₄, THF, 2. Triphosgene, THF; g) NaH, RX, DMF; h) TFA

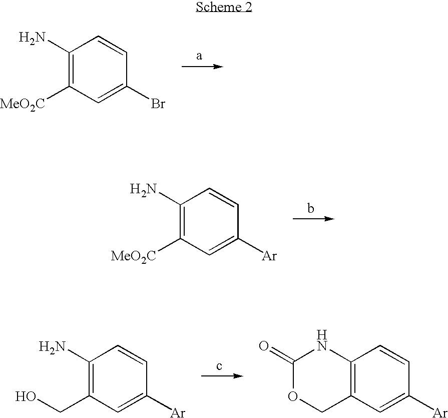

a) ArB(OH)₂, Pd catalyst, aq base, solvent, heat; b) DIBAL, THF; c) Triphosgene, THF

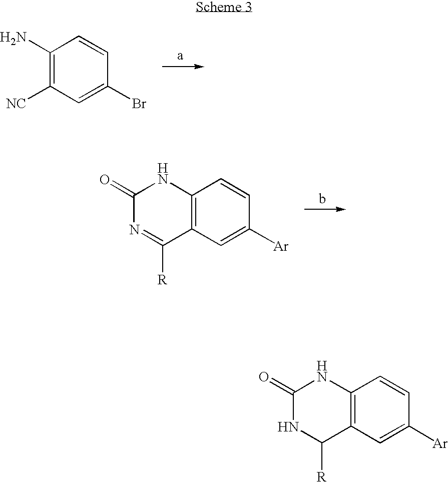

a) RMgBr, THF, then ClCO₂Me; b) NaBH₄, EtOH

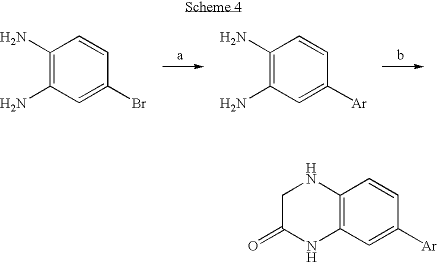

a) ArB(OH)₂, Pd catalyst, aq base, solvent, heat; b) ClCH₂CO₂H, aq (NH₄)₂CO₃, heat

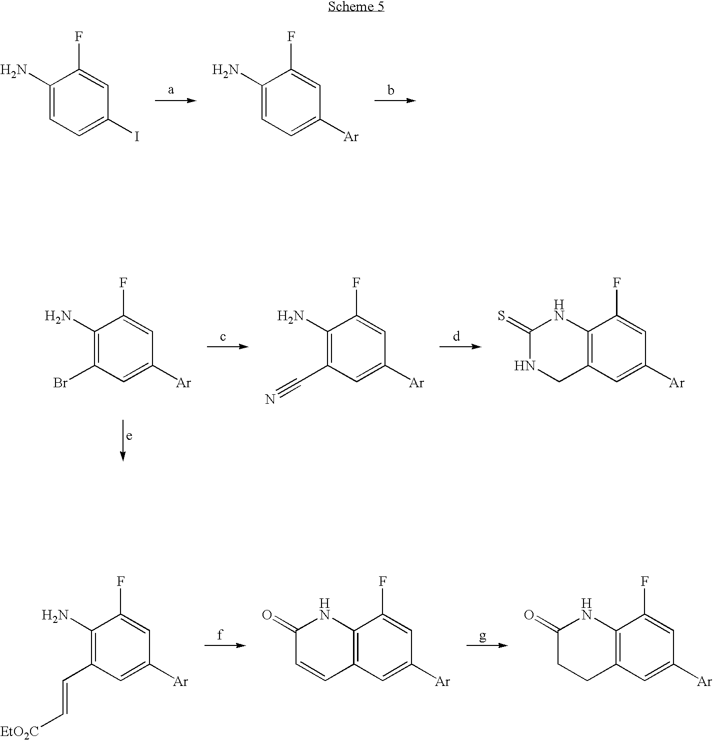

a) ArB(OH)₂, Pd catalyst, aq base, solvent, heat; b) Br₂, AcOH; c) Zn(CN)₂, Pd(PPh₃)₄, DMF, heat; d) 1. LiAlH₄, THF, 2. EtOCS₂K, pyridine, heat; e) Ethyl acrylate, EtNPrⁱ₂, Pd(OAc)₂, P(o-Tol)₃, EtCN, heat; f) Mg, MeOH, heat; g) H₂, Pd/C, EtOH

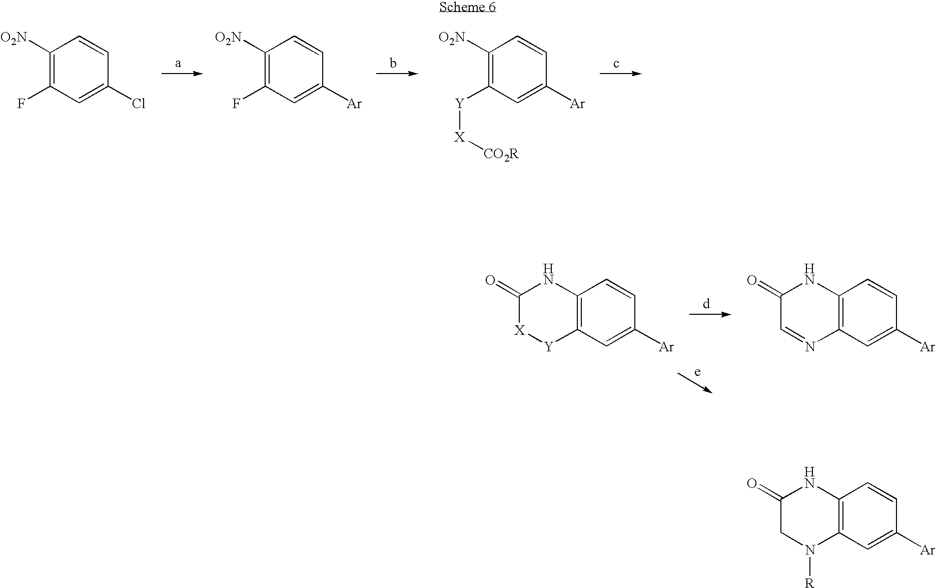

X═CMe₂, CH₂; Y═NH, S, O
   a) ArB(OH)₂, Pd catalyst, aq base, solvent, heat; b) RO₂CXYH, base, solvent, heat; c) H₂, Pd/C, solvent, then cyclise; d) (for X═CH₂, Y═NH) H₂O₂, aq NaOH, heat; e) (for R═CHO) HCO₂H, Ac₂O, heat or (for R═CH₂X) RX, base, heat.

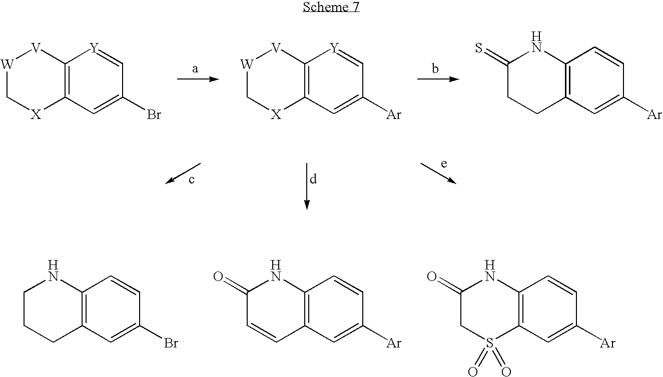

V═CH₂, NH, O; W═CO, SO₂; X═CH₂, S, O; Y═CH, N
   a) ArB(OH)₂, Pd catalyst, aq base, solvent, heat or (dppf)PdCl₂.CH₂Cl₂, ArBr, KOAc, solvent, heat, then starting material, (dppf)PdCl₂, aq base, solvent, heat; b) (for X═CH₂, W═CO, V═NH, Y═CH) Lawesson's reagent, PhMe, heat; c) (for X═CH₂, W═CO, V═NH, Y═CH) LiAlH₄, THF; d) (for X═CH₂, W═CO, V═NH, Y═CH) NBS, (BzO)₂, CHCl₃, heat; e) (for X═S, W═CO, V═NH, Y═CH) mCPBA, dioxane

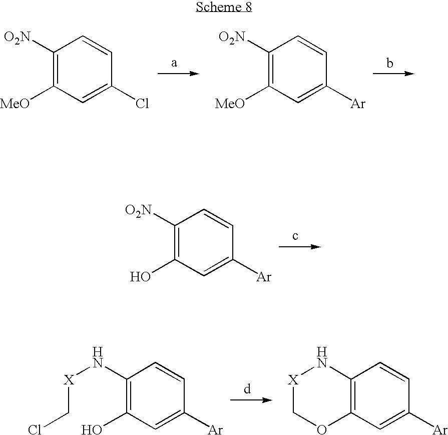

X═CO, SO₂
   a) ArB(OH)₂, Pd catalyst, aq base, solvent, heat; b) LiCl, DMF, reflux; c) 1. Zn, AcOH, 2. ClCH₂XCl, base, solvent; d) K₂CO₃, solvent, heat

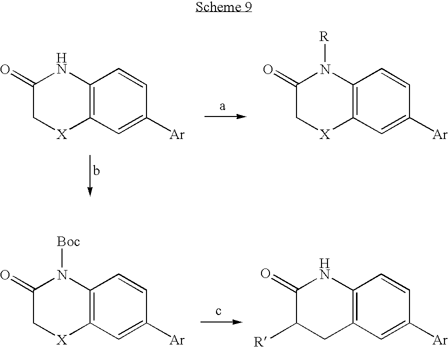

X═S, CH₂, CH₂S
   a) LDA, THF, then RX; b) BOC₂O, base, solvent; c) 1. LDA, THF, −78° C., then R′X, warm to RT, 2. TFA, solvent

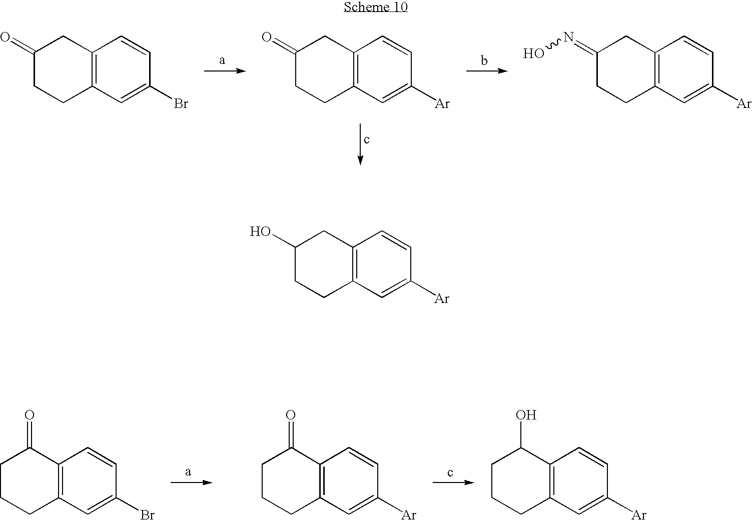

a) ArB(OH)₂, Pd catalyst, aq base, solvent, heat; b) NH₂OH.HCl, pyridine; c) NaBH₄, THF

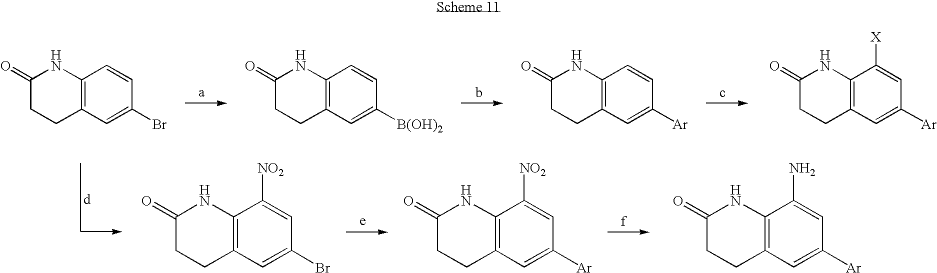

X═Br, CO₂H
   a) 1. NaH, THF, 0° C. 2. n-BuLi, −78° C., 3. B(OMe)₃, RT, 4. H₂O; b) ArBr, Pd catalyst, aq base, solvent, heat; c) 1. NBS, DMF, 60° C. (for X═Br) 2. NaH, THF, then n-BuLi, −78° C., 3. CO₂ (for X═CO₂H); d) conc HNO₃, H₂SO₄; e) ArB(OH)₂, Pd catalyst, aq base, solvent, heat; f) H₂, Pd/C, EtOH

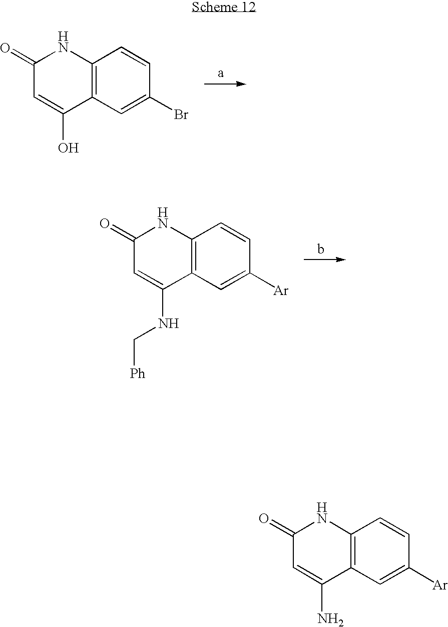

a) 1. PhCH₂NH₂, reflux, 2. ArB(OH)₂, Pd catalyst, aq base, solvent, heat; b) H₂, Pd/C, AcOH, aq HCl

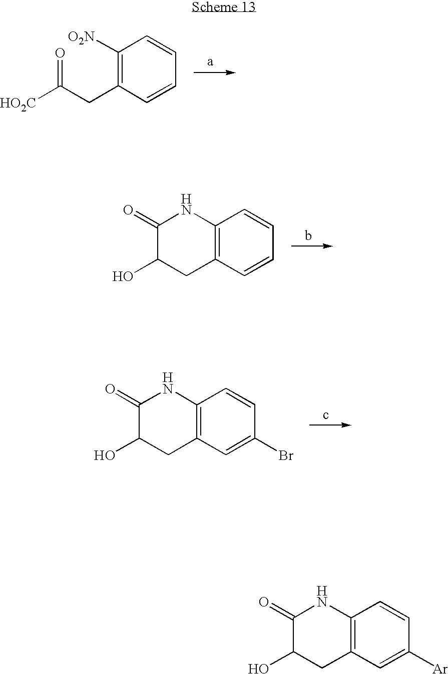

a) 1. NaBH₄, NaOH aq, 2. H₂, Pd/C, MeOH; b) NBS, DMF; c) ArB(OH) 2, Pd catalyst, aq base, solvent, heat

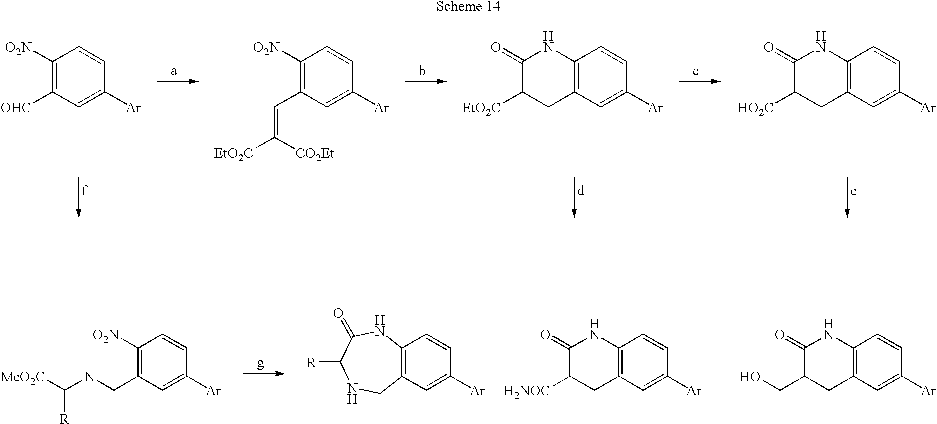

a) CH₂(CO₂Et)₂, TsOH, PhMe, 90° C.; b) 1. NaBH₃CN, conc aq HCl, EtOH/EtOAc, 50° C., 2. H₂, Pd/C, MeOH; c) NaOH, aq MeOH; d) NH₃, MeOH, 80° C.; e) ClCO₂Et, Et₃N, THF, then NaBH₄, aq THF; f) RCH(NH₂)CO₂Me.HCl, MeOH, AcOH, then NaBH₃CN; g) H₂, Pd/C, EtOAc, then Me₃Al, PhMe, 0° C.

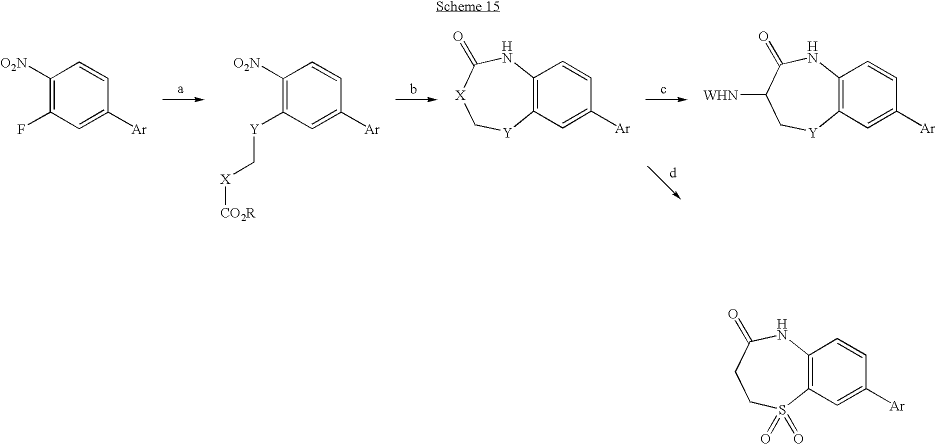

X═CH₂, CH(NHBOC), CH(Me); Y═NH, S, O; W═COR, SO₂R
   a) RO₂CXCH₂YH, base, solvent; b) Zn, AcOH or H₂, MeOH, Pd/C, then EDC, DMF or xylene, heat; c) (for X═CH(NHBOC) TFA/CH₂Cl₂, then EtNPrⁱ₂, WCl, CH₂Cl₂; d) (for Y═S) mCPBA, dioxane.
   For Y═NH, X═CH(Me): a) HO₂CCH(Me)CH₂NH₂, EtNPrⁱ₂, dioxane, heat; b) 1. N-hydroxysuccinimide, DIC, CH₂Cl₂, 2. H₂, MeOH, Pd/C, 3. EtNPrⁱ₂, CH₂Cl₂

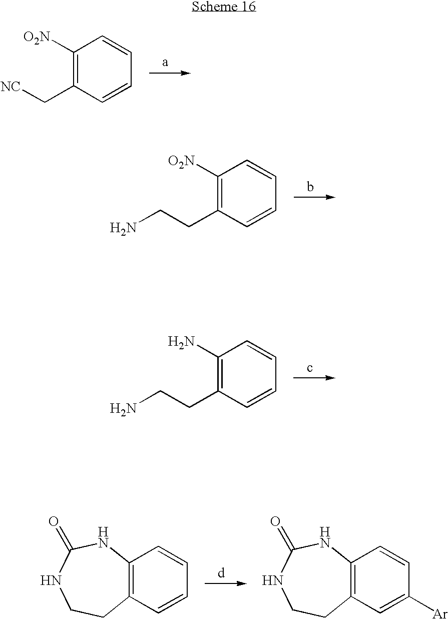

a) BH₃.THF, THF, room temperature; then HCl, 0° C. to room temperature; b) 10% Pd/C, H₂, methanol; c) 1,1′-carbonyldiimidazole, MeCN, 70° C.; d) NBS, DMF; then ArB(OH)₂,
Pd(PPh₃)₄, 2.0M aqueous K₂CO₃, DMF, 80° C.

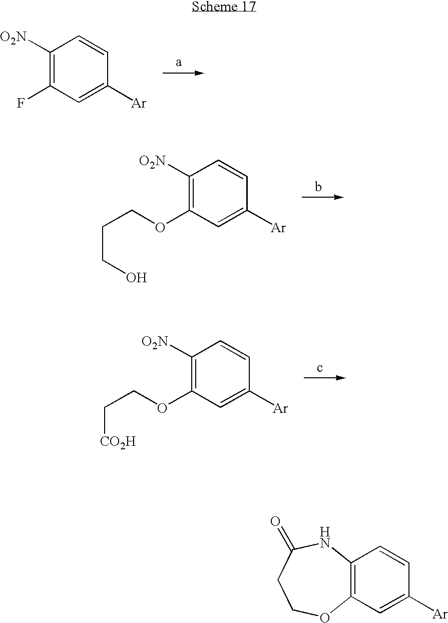

a) NaH, HO(CH₂)₃OH, DMF; b) CrO₃, aq H₂SO₄, acetone; c) H₂, EtOAc, Pd/C, then EDC, DMF

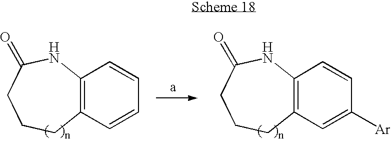

n=1, 2
   a) 1. Br₂, AcOH, H₂SO₄, 2. ArB(OH)₂, Pd catalyst, aq base, solvent, heat

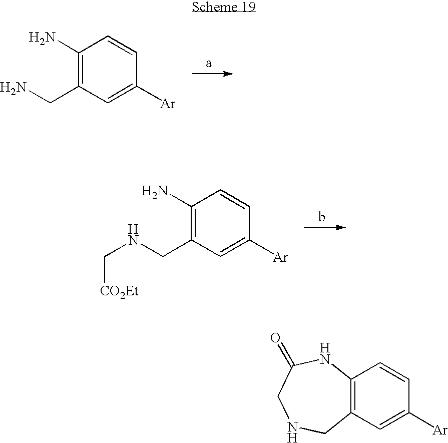

a) BrCH₂CO₂Et, Et₃N, DMF, heat; b) HOBt, PhMe, heat

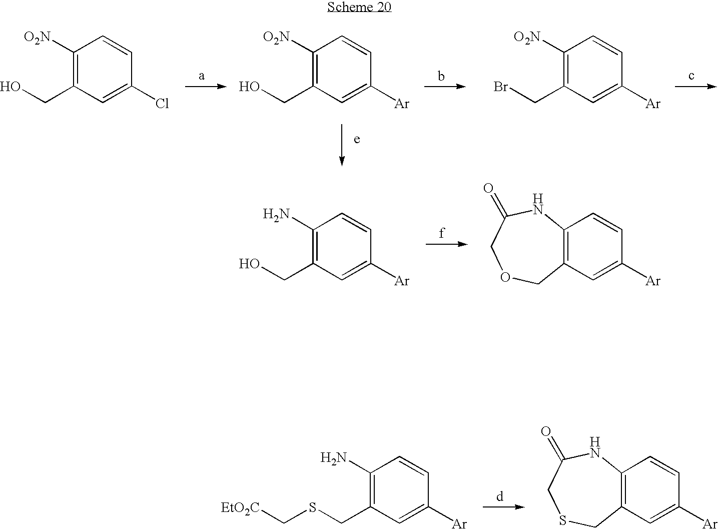

a) ArB(OH)₂, Pd catalyst, aq base, solvent, heat; b) PBr₃, pyridine; c) 1. EtO₂CCH₂SH, K₂CO₃, DMF, 2. Zn, AcOH; d) xylenes, reflux; e) Zn, AcOH; f) 1. ClCH₂COCl, Et₃N, Et₂O, 2. NaH, THF

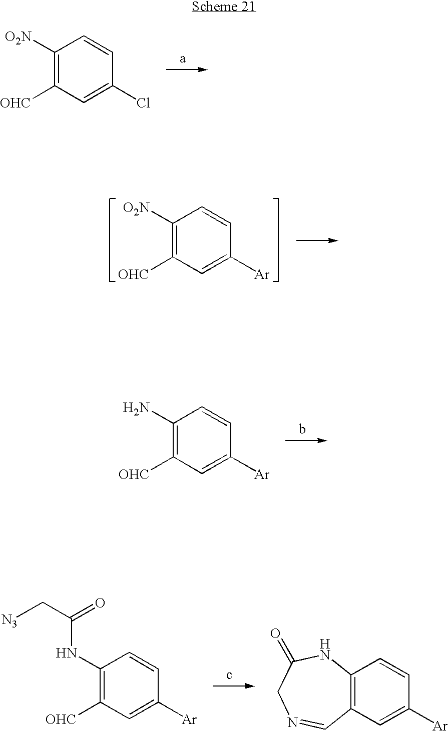

a) ArB(OH)₂, Pd catalyst, aq base, solvent, heat; b) 1. BrCH₂COBr, CH₂Cl₂, then NaOH, 2. NaN₃, acetone; c) PPh₃, aq THF

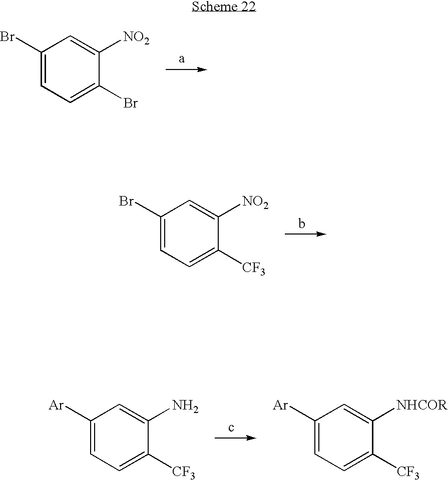

a) MeO₂CCF₂SO₂F, CuI, NMP, 80° C.; b) 1. ArB(OH)₂, Pd catalyst, aq base, solvent, heat, 2. Zn, AcOH; c) (for R═Me) Ac₂O, microwave heating

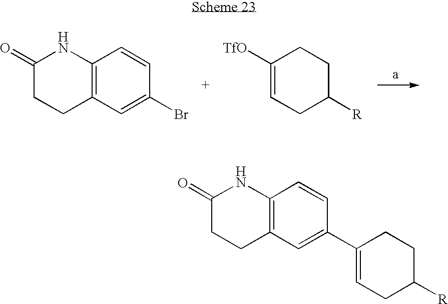

a) Bis(pinacolato)diboron, (dppf)PdCl₂.CH₂Cl₂, aq K₂CO₃, DMF, microwave at 100° C.

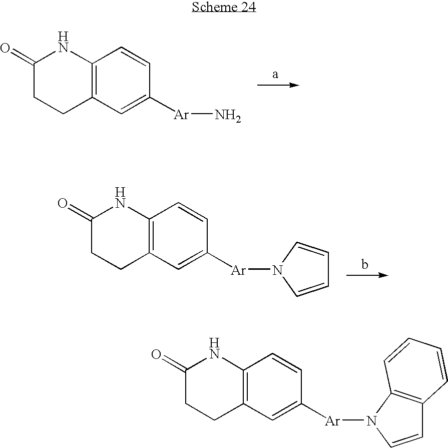

a) 2,5-Dimethoxytetrahydrofuran, AcOH, reflux, 20 min; b) 2,5-dimethoxytetrahydrofuran, AcOH, reflux, 1 h.

### Scheme 25

### Synthesis of Compounds

### EXAMPLE 1

### 6-[4-(Trifluoromethyl)phenyl]-3,4-dihydro-1H-2,1,3-benzothiadiazine 2,2-dioxide

### a) 4-Amino-4′-(trifluoromethyl)-3-biphenylcarbonitrile

A mixture of 4-trifluoromethylbenzeneboronic acid (2.53 g, 13.32 mmol), 2-amino-5-bromobenzonitrile (2.62 g, 13.32 mmol), sodium carbonate (4.6 g, 43.4 mmol) and tetrakis(triphenylphosphine)palladium (0) (440 mg, 0.38 mmol) in water (20 mL) and dimethylformamide (40 mL) was sealed in pressure bottle under argon and heated at 100° C. overnight. The mixture was cooled and diluted with water and extracted twice with ethyl acetate, the combined extracts washed with water and brine, dried and evaporated. The residue was slurried in a mixture of hexane-dichloromethane to give the title compound as a brown solid (2.1 g, 60%). ¹H NMR (400 MHz, D₆-DMSO) δ 7.82-7.87 (m, 3H), 7.72-7.77 (m, 3H), 6.90 (d, J=8.8 Hz, 1H), 6.38 (br s, 2H).

### b) [3-(Aminomethyl)-4′-(trifluoromethyl)-4-biphenylyl]amine

A solution of 4-amino-4′-(trifluoromethyl)-3-biphenylcarbonitrile (2.07 g, 7.9 mmol) in anhydrous tetrahydrofuran (15 mL) was added dropwise to lithium aluminum hydride (36 mL of a 1.0 molar solution in hexanes) cooled to −50° C. Cooling was removed and the mixture was stirred for two hours. The mixture was recooled to −50° C. and treated with a mixture of 2.5 molar sodium hydroxide (1.5 mL) and water (4.25 mL). Cooling was removed and ethyl acetate (100 mL) was added. Stirring continued for 30 minutes, the mixture was filtered through Celite® and evaporated. Filtration through a bed of silica gel eluting with dichloromethane graduating to 20% methanolic ammonia in dichloromethane gave the title compound (1.47 g, 70%). ¹H NMR (400 MHz, D₆-DMSO) δ 7.78 (d, J=8.4 Hz, 2H), 7.70 (d, J=8.4 Hz, 2H), 7.49 (d, J=2.0 Hz, 1H), 7.38 (dd, J=8.1 and 2.3 Hz, 1H), 6.70 (d, J=8.3 Hz, 1H), 5.48 (br s, 2H), 3.70 (s, 2H), 2.30 (br s, 2H).

### c) 6-[4-(Trifluoromethyl)phenyl]-3,4-dihydro-1H-2,1,3-benzothiadiazine 2,2-dioxide

A mixture of [3-(aminomethyl)-4′-(trifluoromethyl)-4-biphenylyl]amine (550 mg, 2.0 mmol) and sulfamide (400 mg, 4.16 mmol) in pyridine (5.0 mL) was heated under reflux for 3 hours. The mixture was evaporated and chromatographed (silica gel, dichloromethane-hexane to 3% methanol in dichloromethane) gave the title compound as crystals from dichloromethane (160 mg, 24%). ¹H NMR (400 MHz, D₆-DMSO) δ 10.35 (s, 1H), 7.70 (d, J=8.4 Hz, 2H), 7.44-7.47 (m, 2H), 7.27 (t, J=7.6 Hz, 1H), 6.70 (d, J=8.4 Hz, 1H), 4.37 (d, J=7.8 Hz, 2H).

### EXAMPLE 2

### 6-[4-(Trifluoromethyl)phenyl]-1H-2,1,3-benzothiadiazine 2,2-dioxide

A solution of potassium permanganate (620 mg, 3.93 mmoles) in water (50 mL) was added to a suspension of 6-[4-(trifluoromethyl)phenyl]-3,4-dihydro-1H-2,1,3-benzothiadiazine 2,2-dioxide (example 1, 0.328 g, 1.00 mmol) in 1.0M sodium hydroxide solution (4.0 mL) and stirred for 20 minutes and filtered; the filter bed was then washed consecutively with water, ethanol 1.0 molar hydrochloric acid, aqueous ethanol and ethyl acetate. Sodium chloride was added to the filtrate and the mixture was extracted twice with ethyl acetate. The combined extracts were dried and evaporated to an orange gum. Trituration with ether afforded the title compound as a cream solid (56 mg, 17%). ¹H NMR (400 MHz, D₆-DMSO) δ 12.50 (br s, 1H), 9.02 (s, 1H), 8.29 (d, J=2.0 Hz, 1H), 8.11 (dd, J=8.6 and 2.0 Hz, 1H), 7.93 (d, J=8.3 Hz, 2H), 7.86 (d, J=8.4 Hz, 2H), 7.21 (d, J=8.6 Hz, 1H).

### EXAMPLE 3

### 6-[4-(Trifluoromethyl)phenyl]-3,4-dihydro-2(1H)-quinazolinone

A solution of [3-(aminomethyl)-4′-(trifluoromethyl)-4-biphenylyl]amine (example 1b, 100 mg, 0.38 mmol) in anhydrous tetrahydrofuran (10 mL) was treated with triphosgene (100 mg, 0.33 mmoles) and stirred for 2 hours. The mixture was poured onto ice, basified with sodium hydrogen carbonate solution and extracted twice with ethyl acetate. The combined extracts were washed with brine, dried and evaporated. Preparative HPLC (20-60% acetonitrile-water-0.1% TFA) gave the title compound as a white solid (75 mg, 67%). ¹H NMR (400 MHz, D₆-DMSO) δ 9.21 (s, 1H), 7.83 (d, J=8.4 Hz, 2H), 7.76 (d, J=8.4 Hz, 2H), 7.55 (m, 2H), 6.89 (m, 2H), 4.39 (s, 2H).

### EXAMPLE 4

### 1-(Phenylmethyl)-6-[4-(trifluoromethyl)phenyl]-3,4-dihydro-2(1H)-quinazolinone

### a) 4-[(Phenylmethyl)amino]-4′-(trifluoromethyl)-3-biphenylcarbonitrile

Sodium hydride (148 mg of a 60% suspension in oil, 3.7 mmol) was added to a solution of 4-amino-4′-(trifluoromethyl)-3-biphenylcarbonitrile (example 1a, 700 mg, 2.67 mmol) in anhydrous tetrahydrofuran (10 mL) in a nitrogen atmosphere at room temperature. The mixture was stirred for 10 minutes the benzyl bromide (320 uL, 2.67 mmol) was added dropwise. Stirring continued for two hours, the mixture was poured into water and extracted twice with ethyl acetate. The combined extracts were washed with brine, dried and evaporated. Flash chromatography (silica gel, hexane to 20% ethyl acetate in hexane gave the title compound (286 mg, 30%). ¹H NMR (400 MHz, CDCl₃) δ 7.65-7.68 (m, 3H), 7.56-7.59 (m, 3H), 7.27-7.39 (m, 5H) 6.74 (d, J=8.8 Hz, 1H), 5.18 (br t, 1H), 4.50 (d, J=5.6 Hz, 2H).

### b) 1-(Phenylmethyl)-6-[4-(trifluoromethyl)phenyl]-3,4-dihydro-2(1H)-quinazolinone

A solution of 4-[(phenylmethyl)amino]-4′-(trifluoromethyl)-3-biphenylcarbonitrile (286 mg, 0.81 mmol) in anhydrous tetrahydrofuran (10 mL) was added dropwise to lithium aluminum hydride (3.25 mL of a 1.0 molar solution in hexanes) cooled to −50° C. Cooling was removed and the mixture was stirred for sixteen hours. The mixture was recooled to −50° C. and treated with a mixture of 2.5 molar sodium hydroxide (0.15 mL) and water (0.4 mL). Cooling was removed and ethyl acetate (10 mL) was added. Stirring continued for 30 minutes, the mixture was filtered through Celite® and evaporated to give crude [3-(aminomethyl)-4′-(trifluoromethyl)-4-biphenylyl](phenylmethyl)amine (240 mg).

A solution of this diamine (120 mg, 0.33 mmol) in anhydrous tetrahydrofuran (10 mL) was treated with triphosgene (100 mg, 0.33 mmoles) and stirred for 5 hours. The mixture was poured onto ice, basified with sodium hydrogen carbonate solution and extracted twice with ethyl acetate. The combined extracts were washed with brine, dried and evaporated. Flash chromatography (silica gel, hexane to 30% ethyl acetate-hexane) gave the title compound as a white solid (20 mg, 16%). ¹H NMR (400 MHz, D₆-DMSO) δ 8.12 (t, J=7.8 Hz, 1H), 7.83 (d, J=8.3 Hz, 2H), 7.77 (d, J=8.4 Hz, 2H), 7.64 (m, 1H), 7.55 (dd, J=8.6 and 2.0 Hz, 1H), 7.47 (d, J=7.6 Hz, 2H), 7.37 (t, J=7.2 Hz, 2H), 7.28 (d, J=7.3 Hz, 1H), 6.82 (d, J=8.8 Hz, 1H), 5.05 (s, 2H), 4.63 (d, J=7.5 Hz, 2H).

### EXAMPLE 5

### 6-[4-(Trifluoromethyl)phenyl]-3,4-dihydro-2(1H)-quinazolinethione

A mixture of [3-(aminomethyl)-4′-(trifluoromethyl)-4-biphenylyl]amine (example 1b, 140 mg, 0.52 mmol) and potassium ethyl xanthate (84 mg, 0.52 mmol) in pyridine (2.0 mL) was heated under reflux for 2 hours. The mixture was cooled, partitioned between ethyl acetate and water, the aqueous mixed with brine and extracted twice with ethyl acetate. The combined extracts were washed twice with 1 molar hydrochloric acid, dried and evaporated to a solid. Recrystallization from dichloromethane afforded the title compound (32 mg, 20%). ¹H NMR (400 MHz, D₆-DMSO) δ 10.55 (s, 1H), 8.73 (s, 1H), 7.84 (d, J=8.3 Hz, 2H), 7.78 (d, J=8.3 Hz, 2H), 7.59 (d, J=8.3 Hz, 1H), 7.54 (s, 1H), 7.03 (d, J=8.1 Hz, 1H), 4.45 (s, 2H).

### EXAMPLE 6

### 6-[4-(Trifluoromethyl)phenyl]-1,4-dihydro-2H-3,1-benzoxazin-2-one

### a) Methyl 4-amino-4′-(trifluoromethyl)-3-biphenylcarboxylate

A mixture of 4-trifluoromethylbenzeneboronic acid (440 mg, 2.31 mmol), methyl 2-amino-5-bromobenzoate (535 mg, 2.31 mmol), sodium carbonate (730 mg, 6.9 mmol) and tetrakis(triphenylphosphine)palladium (0) (80 mg, 0.07 mmol) in water (2 mL) and dimethylformamide (5 mL) was sealed in pressure bottle under argon and heated at 100° C. overnight. The mixture was cooled and diluted with water and extracted twice with ethyl acetate, the combined extracts washed with water and brine, dried and evaporated. Flash chromatography (silica gel, hexane to 10% ethyl acetate in hexane) gave the title compound (600 mg, 88%). ¹H NMR (400 MHz, D₆-DMSO) δ 8.13 (d, J=2.3 Hz, 1H), 7.84 (d, J=8.6 Hz, 2H), 7.79 (d, J=8.3 Hz, 2H), 7.76 (dd, J=8.6 and 2.3 Hz, 1H), 6.9-7.0 (m, 3H), 3.90 (s, 3H).

### b) [4-Amino-4′-(trifluoromethyl)-3-biphenylyl]methanol

Diisobutylaluminum hydride (6.0 mL of a 1.0 molar solution in hexanes) was added dropwise to a solution of methyl 4-amino-4′-(trifluoromethyl)-3-biphenylcarboxylate (600 mg, 2.0 mmol) in tetrahydrofuran (30 mL) under nitrogen atmosphere at −70° C. The mixture was stirred at ambient temperature for six hours before being treated with methanol (3.0 mL), water (2.0 mL) and ethyl acetate (30 mL). After stirring for 30 minutes, the mixture was evaporated onto silica gel and chromatographed (silica gel, hexane to 50% ethyl acetate in hexane) to give recovered stating material (150 mg) plus the title compound (250 mg, 47%). ¹H NMR (400 MHz, D₆-DMSO) δ 7.77 (d, J=8.3 Hz, 2H), 7.71 (d, J=8.3 Hz, 2H), 7.51 (d, J=2.1 Hz, 1H), 7.41 (dd, J=8.4 and 2.3 Hz, 1H), 6.73 (d, J=8.4 Hz, 1H), 5.26 (br s, 2H), 5.11 (t, J=5.5 Hz, 1H), 4.45 (d, J=5.3 Hz, 2H).

### c) 6-[4-(Trifluoromethyl)phenyl]-1,4-dihydro-2H-3,1-benzoxazin-2-one

Triphosgene was added to a solution of [4-amino-4′-(trifluoromethyl)-3-biphenylyl]methanol (94 mg, 0.35 mmol) in tetrahydrofuran (10 mL) and the mixture was stirred at ambient temperature overnight. The mixture was poured onto ice and neutralized with sodium hydrogen carbonate solution, diluted with brine and extracted twice with ethyl acetate. The combined extracts were dried and evaporated to a white solid. The solid was slurried in diethyl ether, collected and washed with diethyl ether and hexane to give the title compound (45 mg, 44%). ¹H NMR (400 MHz, D₆-DMSO) δ S 10.34 (s, 1H), 7.85 (d, J=8.3 Hz, 2H), 7.791 (d, J=8.5 Hz, 2H), 7.60-7.70 (m, 2H), 7.0 (d, J=8.2 Hz, 1H), 5.39 (s, 2H).

### EXAMPLE 7

### ±4-Methyl-6-[4-(trifluoromethyl)phenyl]-3,4-dihydro-2(1H)-quinazolinone

4-Amino-4′-(trifluoromethyl)-3-biphenylcarbonitrile (example 1a, 0.168 g, 0.640 mmol) in THF (4.0 mL) was added dropwise to a solution of methylmagnesium bromide (2.6 mL, 3.0 M in ethyl ether). The reaction was allowed to stir at room temperature for one hour and then heated to 50° C. for 1.5 h. The reaction was cooled to 0° C. and methylchloroformate (0.59 mL, 7.64 mmol) was added dropwise. The reaction was heated to 50° C. for 12 hours, cooled and quenched with 6N HCl. The crude mixture was next neutralized with NaHCO₃ (sat) and extracted with ethyl acetate twice, organics were combined and dried over MgSO₄, filtered and concentrated under reduced pressure. The crude product (0.145 g) was dissolved in ethanol (3.6 mL) and sodium borohydride (0.072 g, 1.903) added portion wise. The reaction was allowed to stir for 30 min. and then quenched with acetic acid. The reaction mixture was concentrated and column chromotography (1:1 hexane:ethyl acetate to 100% ethyl acetate) provided the title compound (6.5 mg, 4%) as a yellow solid. ¹H NMR (400 MHz, D₆-DMSO) δ 8.15 (bs, 1H), 7.80 (dd, 4H), 7.52 (s, 1H), 7.01 (s, 1H), 6.89 (d, J=8.8 Hz, 1H), 6.55 (bs, 1H), 4.59 (m, 1H), 1.38 (d, 3H).

### EXAMPLE 8

### 7-[4-(Trifluoromethyl)phenyl]-3,4-dihydro-2(1H)-quinoxalinone

### a) 4′-(Trifluoromethyl)-3,4-biphenyldiamine

A mixture of 4-bromo-1,2-benzenediamine (0.94 g, 5.03 mmol), 4-(trifluoromethyl)phenylboronic acid (1.90 g, 10.0 mmol), tetrakis(triphenylphosphine)palladium (0) (0.289 g, 0.25 mmol), 2M aqueous potassium carbonate (20 mL, 40 mmol) and dimethylformamide (20 mL) was stirred under argon at 90° C. for 20 h, then cooled, poured into water (200 mL) and extracted with ethyl acetate. The extracts were washed with dilute aqueous potassium carbonate, brine and dried (Na₂SO₄), then the solvent removed under reduced pressure. The residue was chromatographed (silica gel, 50-100% ethyl acetate/hexane) to give the title compound (0.964 g, 76%) as a solid. ¹H NMR (400 MHz, D₆-DMSO) δ 7.69 (s, 4H), 6.93 (d, J=2.2 Hz, 1H), 6.82 (dd, J=8.0, 2.2 Hz, 1H), 6.61 (d, J=8.0 Hz, 1H), 4.72 (br s, 4H).

### b) 7-[4-(Trifluoromethyl)phenyl]-3,4-dihydro-2(1H)-quinoxalinone

A mixture of 4′-(trifluoromethyl)-3,4-biphenyldiamine (0.252 g, 1.00 mmol), chloroacetic acid (0.098 g, 1.04 mmol), and aqueous ammonium carbonate (0.1M, 5.2 mL, 0.52 mmol) was stirred while heating under reflux for 1 h, then cooled, acidified with aqueous HCl and extracted with ethyl acetate. The extracts were washed with aqueous HCl, aqueous NaHCO₃, brine and dried (MgSO₄), then the solvent removed under reduced pressure and the residue chromatographed (silica gel, 2-10% methanol/dichloromethane). The product was triturated with ether and dried to give the title compound (0.031 g, 11%) as an off-white powder. ¹H NMR (400 MHz, D₆-DMSO) δ 10.37 (s, 1H), 7.75 (d, J=8.5 Hz, 2H), 7.71 (d, J=8.4 Hz, 2H), 7.19 (dd, J=8.2, 2.1 Hz, 1H), 7.09 (d, J=2.1 Hz, 1H), 6.77 (d, J=8.2 Hz, 1H), 6.28 (s, 1H), 3.81 (d, J=1.7 Hz, 2H).

### EXAMPLE 9

### 4-Methyl-6-[4-trifluoromethyl)phenyl]-2(1H)-quinazolinone

Purification of a portion of the crude material of example 7 prior to sodium borohydride reduction by prep HPLC provided the title compound (6%) as a white solid. ¹H NMR (400 MHz, MeOD) δ 8.31 (d, J=1.8 Hz, 1H), 8.13 (dd, J=1.9, 6.7 Hz, 1H), 7.93 (m, 4H), 7.81 (d, J=8.2 Hz, 1H), 7.47 (d, J=8.6 Hz, 1H), 2.90 (s, 3H).

### EXAMPLE 10

### 4-Ethyl-6-[4-(trifluoromethyl)phenyl]-3,4-dihydro-2(1H)-quinazolinone

4-Amino-4′-(trifluoromethyl)-3-biphenylcarbonitrile (0.154 g, 0.587 mmol) in THF (4.0 mL) was added dropwise to a solution of ethylmagnesium bromide (1.9 mL, 3.0 M in ethyl ether) in THF (1.0 mL) at 0° C. The reaction was then heated to 50° C. for 2 hours and then cooled to 0° C. and methylchloroformate (0.50 mL, 6.45 mmol) was added dropwise. The reaction was heated to 50° C. for 12 hours and then cooled and quenched with 1N HCl. The crude mixture was next neutralized with NaHCO₃ (sat) and twice extracted with ethyl acetate, organics combined and dried over MgSO₄, filtered and concentrated under reduced pressure. Crude ¹H NMR (400 MHz, D₆-DMSO) δ 9.81 (bs, 1H), 8.76 (bs, 1H), 7.88 (d, 2H), 7.85 (s, 1H), 7.74 (d, 2H), 7.57 (d, J=9.8 Hz, 1H), 6.94 (d, J=8.5 Hz, 1H), 5.34 (q, 1H), 1.71 (d, 3H) A portion of crude reaction mixture (0.049 g, 0.154 mmol) was dissolved in ethanol (3.0 mL) and sodium borohydride (0.070 g, 1.850) added portion wise. The reaction was allowed to stir for 30 min. and then quenched with acetic acid. The reaction mixture was concentrated and prep HPLC provided the title compound (6.9 mg, 14%) as a yellow solid. ¹H NMR (400 MHz, D₆-DMSO) δ 9.21 (s, 1H), 7.80 (m, 4H), 7.55 (m, 2H), 7.10 (s, 1H), 6.80 (d, 1H), 4.42 (m, 1H), 1.67 (q, 2H), 0.82 (t, 3H).

### EXAMPLE 11

### 8-Fluoro-6-[4-(trifluoromethyl)phenyl]-3,4-dihydro-2(1H)-quinazolinethione

### a) 3-Fluoro-4′-(trifluoromethyl)-4-biphenylamine

To 2-fluoro-4-iodoaniline (2.4 g, 10 mmol) and [4-(trifluoromethyl)phenyl]boronic acid (2.0 g, 10.5 mmol) in a (1:1) solution of dimethylformamide and aq. 2M potassium carbonate (50 mL) was added with stirring tetrakis(triphenylphosphine)palladium (0) (0.58 g, 0.5 mmol). The reaction was purged with nitrogen then stirred for 18 h at 100° C. The reaction was cooled, evaporated under vacuum, taken up in ethyl acetate, washed with water, brine, dried (Na₂SO₄), filtered and concentrated under vacuum. Purification by flash chromatography on silica gel (20% ethyl acetate/hexanes) gave the title compound (1.92 g, 74%) as an off-white solid: MS (ES) m/e 256.2 (M+H)⁺.

### b) [3-Bromo-5-fluoro-4′-(trifluoromethyl)-4-biphenylyl]amine

To a stirred solution of 3-fluoro-4′-(trifluoromethyl)-4-biphenylamine (1.5 g, 5.9 mmol) in acetic acid (40 mL) was added dropwise bromine (0.330 mL, 6.4 mmol). The reaction was stirred at 40° C. for 4 h then evaporated to dryness under vacuum. The residue was taken up in ethyl acetate, washed with 1M aq sodium carbonate, brine, dried (Na₂SO₄) and evaporated under vacuum. Purification by flash chromatography on silica gel (10% ethyl acetate/hexanes) gave the title compound (1.85 g, 94%) as a white crystalline solid: MS (ES) m/e 334.0 (M+H)⁺.

### c) 4-Amino-5-fluoro-4′-(trifluoromethyl)-3-biphenylcarbonitrile

To a stirred solution of [3-bromo-5-fluoro-4′-(trifluoromethyl)-4-biphenylyl]amine (1.85 g, 5.5 mmol) and zinc cyanide (1.0 g, 8.5 mmol) in dimethylformamide (30 mL) was added tetrakis(triphenylphosphine)palladium (0) (0.64 g, 0.6 mmol). The reaction was purged with nitrogen and stirred at 100° C. for 24 h. After cooling to room temperature, the reaction was evaporated to dryness under vacuum. The residue was taken up in ethyl acetate, washed with water, filtered to remove insolubles, dried (Na₂SO₄), and concentrated under vacuum. Purification by flash chromatography on silica gel (15% ethyl acetate/hexanes) gave the title compound (1.52 g, 98%) as a white solid: MS (ES) m/e 281.4 (M+H)⁺.

### d) 8-Fluoro-6-[4-(trifluoromethyl)phenyl]-3,4-dihydro-2(1H)-quinazolinethione

To a stirred solution of 4-amino-5-fluoro-4′-(trifluoromethyl)-3-biphenylcarbonitrile (0.38 g, 1.4 mmol) in tetrahydrofuran (5 mL) at −78° C. under nitrogen was added dropwise 1M lithium aluminum hydride in tetrahydrofuran (5.5 mL, 5.5 mmol). The reaction was allowed to warm to room temperature and stirred for 2 h, cooled to 0° C. and with vigorous stirring slowly quenched sequentially with water (0.2 mL), 15% aq. sodium hydroxide (0.2 mL) and water (0.6 mL). The reaction was allowed to warm to room temperature and stirred for an additional 1 h. The resulting suspension was filtered through a pad of Celite®, rinsed with tetrahydrofuran, and the filtrate evaporated to dryness under vacuum. The oil which remained was taken up in pyridine (10 mL) and treated with O-ethylxanthic acid potassium salt (0.220 g, 1.4 mmol). The reaction was refluxed (120° C. oil bath) for 18 h, cooled to room temperature and evaporated to dryness under vacuum. The residue which remained was taken up in ethyl acetate, washed with 1M hydrochloric acid, brine, dried (MgSO₄), filtered and concentrated under vacuum. Purification by flash chromatography on silica gel (40 to 80% ethyl acetate/hexanes) gave, after trituration with hexanes, followed by (1:1) acetonitrile/water, filtration and drying under vacuum, the title compound (135 mg, 30%) as a off-white solid: ¹H NMR (400 MHz, DMSO-d₆) δ ppm 10.44 (s, 1H), 8.90 (s, 1H), 7.89 (d, J=8.1 Hz, 2H), 7.79 (d, J=8.1 Hz, 2H), 7.58 (dd, J=11.8, 1.6 Hz, 1H), 7.44 (s, 1H), 4.47 (s, 2H); MS (ES) m/e 327.0 (M+H)⁺.

### EXAMPLE 12

### 6-(4-Trifluoromethylphenyl)-3,4-dihydro-1H-quinolin-2-one

A mixture of 6-bromo-3,4-dihydro-1H-quinolin-2-one (0.226 g, 1.00 mmol), 4-(trifluoromethyl)phenylboronic acid (0.380 g, 2.0 mmol), tetrakis(triphenylphosphine)palladium (0) (0.065 g, 0.056 mmol), 2M aqueous potassium carbonate (5 mL, 10 mmol) and dioxane (5 mL) was stirred under argon at 90° C. for 20 h, then cooled, poured into 0.4M aqueous HCl (50 mL) and extracted with ethyl acetate. The extracts were washed with water, brine and dried (MgSO₄), then the solvent removed under reduced pressure and the residue chromatographed (silica gel, 20-80% ethyl acetate/hexane) to give the title compound (0.249 g, 85%) as a cream solid. ¹H NMR (400 MHz, D₆-DMSO) δ 10.23 (s, 1H), 7.86 (d, J=8.2 Hz, 2H), 7.78 (d, J=8.4 Hz, 2H), 7.60 (s, 1H), 7.55 (dd, J=8.2, 2.1 Hz, 1H), 6.98 (d, J=8.2 Hz, 1H), 2.97 (t, J=7.5 Hz, 2H), 2.51 (partially obscured by D₅-DMSO, 2H).

### EXAMPLE 13

### 6-[4-(Trifluoromethyl)phenyl]-3,4-dihydro-2(1H)-quinoxalinone

### a) 3-Fluoro-4-nitro-4′-(trifluoromethyl)biphenyl

A mixture of 4-chloro-2-fluoronitrobenzene (1.40 g, 7.97 mmol), 4-(trifluoromethyl)phenylboronic acid (1.54 g, 8.11 mmol), tetrakis(triphenylphosphine)palladium (0) (0.150 g, 0.13 mmol), sodium carbonate (2.55 g, 24.1 mmol), water (3 mL) and dioxane (18 mL) was heated under argon at 100° C. in a sealed tube for 2 h, then cooled, poured into water (200 mL) and extracted with ethyl acetate. The extracts were washed with water and brine and dried (MgSO₄), then the solvent removed under reduced pressure. The residue was recrystallised (ether/hexane) to give the title compound (0.940 g, 41%) as a white solid. ¹H NMR (400 MHz, D₆-DMSO) δ 8.29 (t, J=8.2 Hz, 1H), 8.07 (dd, J=12.8, 1.7 Hz, 1H), 8.06 (d, J=8.1 Hz, 2H), 7.90 (d, J=8.3 Hz, 2H), 7.86 (dd, J=8.8, 1.4 Hz, 1H).

### b) 1,1-Dimethylethyl N-[4-nitro-4′-(trifluoromethyl)-3-biphenylyl]glycinate

A mixture of 1,1-dimethylethyl glycinate hydrochloride (1.20 g, 7.16 mmol), 3-fluoro-4-nitro-4′-(trifluoromethyl)biphenyl (1.00 g, 3.51 mmol), triethylamine (1.00 mL, 7.17 mmol) and dioxane (10 mL) was stirred at 100° C. under argon for 6 h, then cooled and partitioned between water and ethyl acetate. The extracts were washed with water and brine, dried (MgSO₄), then the solvent removed under reduced pressure and the residue chromatographed (silica gel, 5-30% ethyl acetate/hexane) to give the title compound (1.29 g, 93%) as a solid. ¹H NMR (400 MHz, D₆-DMSO) δ 8.46 (br t, J=5.3 Hz, 1H), 8.22 (d, J=8.9 Hz, 1H), 8.00 (d, J=8.2 Hz, 2H), 7.88 (d, J=8.3 Hz, 2H), 7.19 (d, J=1.8 Hz, 1H), 7.11 (dd, J=8.9, 1.8 Hz, 1H), 4.34 (d, J=5.4 Hz, 2H), 1.46 (s, 9H).

### c) 6-[4-(Trifluoromethyl)phenyl]-3,4-dihydro-2(1H)-quinoxalinone

A solution of 1,1-dimethylethyl N-[4-nitro-4′-(trifluoromethyl)-3-biphenylyl]glycinate (0.091 g, 0.230 mmol) in methanol/ethyl acetate (1:1, 8 mL) was stirred with palladium-on-charcoal (5%, 0.035 g) under 1 atm hydrogen for 3 h, then the reaction flushed with nitrogen and filtered though filter agent. The solvent was removed from the filtrate under reduced pressure to leave the crude aniline as a yellow solid. A solution of this aniline in ether (5 mL) and 4M hydrogen chloride in dioxane (2 mL) was stirred at room temperature for 60 h, then partitioned between water and ethyl acetate. The extracts were washed with water and brine, dried (MgSO₄) and the solvent removed under reduced pressure. The residual solid was triturated (ether/hexane) to give the title compound (0.048 g, 71%) as a cream solid. ¹H NMR (400 MHz, D₆-DMSO) δ 10.39 (s, 1H), 7.77 (d, J=9.2 Hz, 2H), 7.75 (d, J=9.2 Hz, 2H), 7.02 (d, J=1.9 Hz, 1H), 6.98 (dd, J=8.0, 2.0 Hz, 1H), 6.84 (d, J=8.0 Hz, 1H), 6.13 (s, 1H), 3.79 (d, J=1.7 Hz, 2H).

### EXAMPLE 14

### 6-[4-(Trifluoromethyl)phenyl]-3,4-dihydro-2(1H)-quinolinethione

A solution of 6-(4-trifluoromethylphenyl)-3,4-dihydro-1H-quinolin-2-one (example 12, 0.093 g, 0.319 mmol) and 2,4-bis(4-methoxyphenyl)-1,3-dithia-2,4-diphosphetane-2,4-disulfide (Lawesson's reagent, 0.065 g, 0.160 mmol) in toluene (8 mL) was heated under reflux under nitrogen for 0.5 h, then cooled and loaded onto a silica gel column. The product was eluted with 10-50% ethyl acetate/hexane to give the title compound (0.080 g, 82%) as a solid. ¹H NMR (400 MHz, D₆-DMSO) δ 12.33 (br s, 1H), 7.89 (d, J=8.2 Hz, 2H), 7.80 (d, J=8.3 Hz, 2H), 7.66 (d, J=1.8 Hz, 1H), 7.63 (dd, J=8.2, 2.1 Hz, 1H), 7.22 (d, J=8.2 Hz, 1H), 2.99 (td, J=7.5, 1.8 Hz, 2H), 2.90 (t, J=7.6 Hz, 2H).

### EXAMPLE 15

### 7-[4-(Trifluoromethyl)phenyl]-2H-1,4-benzothiazin-3(4H)-one

A mixture of 7-bromo-2H[1,4]-benzothiazin-3(4H)-one (2.5 g, 10.24 mmol), 4-(trifluoromethyl)benzeneboronic acid (2.88 g, 15.36 mmol), tetrakis(triphenylphosphine)palladium(0) (1.18 g, 1.024 mmol), and 2M aqueous potassium carbonate (28.16 mL, 56.32 mmol) in dimethylformamide (30 mL) was heated for 18 hours at 100° C. The reaction was cooled and filtered through Celite®. The filtrate was diluted with ethyl acetate, washed with water and saturated sodium chloride, dried (Na₂SO₄), and partially concentrated in vacuo. The ethyl acetate solution was placed in the freezer overnight and the resulting precipitate was filtered and dried in a Buchner funnel to give the title compound (1.37 g, 43%) as a tan solid. MS (ES+) m/e 310 [M+H]⁺.

### EXAMPLE 16

### 7-[4-(Trifluoromethyl)phenyl]-2H-1,4-benzoxazin-3(4H)-one

### a) 3-Methoxy-4-nitro-4′-(trifluoromethyl)biphenyl

A mixture of 4-chloro-2-methoxy-1-nitrobenzene (6.76 g, 36.04 mmol), 4-(trifluoromethyl)benzeneboronic acid (10.27 g, 54.06 mmol), tetrakis(triphenylphosphine)palladium (0) (2.08 g, 1.802 mmol), and 2M aqueous potassium carbonate (100 mL, 200.0 mmol) in dimethylformamide (100 mL) was heated for 3.0 hours at 100° C. The reaction was cooled and filtered through Celite®. The filtrate was diluted with ethyl acetate, washed with water and saturated sodium chloride, dried (Na₂SO₄), and purified by flash chromatography on silica gel (9:1 hexanes:EtOAc) give the title compound (9.37 g, 88%) as a pale yellow solid. MS (ES+) m/e 298 [M+H]⁺.

### b) 3-Hydroxy-4-nitro-4′-(trifluoromethyl)biphenyl

A mixture of 3-methoxy-4-nitro-4′-(trifluoromethyl)biphenyl (3.60 g, 12.11 mmol) and lithium chloride (1.54 g, 36.33 mmol) in dimethylformamide (36 mL) was refluxed for 3.5 hours. The reaction was cooled, diluted with water, acidified to pH 1.0 with 1N HCl, and the resulting precipitate filtered and dried in a Buchner funnel to give the title compound (3.22 g, 94%) as an orange powder. MS (ES+) m/e 284 [M+H]⁺.

### c) 4-Amino-3-hydroxy-4′-(trifluoromethyl)biphenyl

A mixture of 3-hydroxy-4-nitro-4′-(trifluoromethyl)biphenyl (3.20 g, 11.31 mmol) and zinc powder (7.40 g, 113 1 mmol) in acetic acid (60 mL) was stirred for 3.0 hours at room temperature. The reaction was filtered through Celite®e, concentrated in vacuo, and purified by flash chromatography on silica gel (60:40 hexanes:ethyl acetate) to give the title compound (1.71 g, 60%) as a brown solid. MS (ES+) m/e 254 [M+H]⁺.

### d) 2-Chloro-N-[3-hydroxy-4′-(trifluoromethyl)-4-biphenylyl]acetamide

4-Amino-3-hydroxy-4′-(trifluoromethyl)biphenyl (0.317 g, 1.252 mmol) and sodium bicarbonate (0.258 g, 3.07 mmol) were dissolved in a mixture of methyl isobutylketone (3.0 mL) and water (2.5 mL). A solution of chloroacetyl chloride (0.158 g, 1.40 mmol) in methyl isobutylketone (3.0 mL) was slowly added and the reaction was refluxed for 2.5 hours. The reaction was cooled, diluted with water, extracted into ethyl acetate, washed with water and brine, dried (Na₂SO₄), and purified by flash chromatography to give the title compound as a pinkish solid. MS (ES+) m/e 330 [M+H]⁺.

### e) 7-[4-(Trifluoromethyl)phenyl]-2H-1,4-benzoxazin-3(4H)-one

A mixture of 2-chloro-N-[3-hydroxy-4′-(trifluoromethyl)-4-biphenylyl]acetamide (0.26 g, 0.79 mmol) and potassium carbonate (0.109 g, 0.79 mmol) in dimethylformamide (10 mL) was heated at 80° C. for 3.0 hours, cooled, and diluted with water. The resulting precipitate was filtered and dried to give the title compound (0.217 g, 2 steps, 59%) as a tan solid. MS (ES+) m/e 294 [M+H]⁺.

### EXAMPLE 17

### 6-[4-(Trifluoromethyl)phenyl]-2(1H)-quinolinone

A solution of 6-(4-trifluoromethylphenyl)-3,4-dihydro-1H-quinolin-2-one (example 12, 0.114 g, 0.391 mmol), N-bromosuccinimide (0.077 g, 0.433 mmol) and benzoyl peroxide (˜1 mg) in chloroform (5 mL) was heated under reflux under nitrogen for 6 h, then cooled and the solvent removed under reduced pressure. The residue was chromatographed (silica gel, 40-100% ethyl acetate/hexane) and the product triturated (ether/hexane) to give the title compound (0.021 g, 19%) as a solid. ¹H NMR (400 MHz, D₆-DMSO) δ 11.89 (br s, 1H), 8.11 (d, J=1.1 Hz, 1H), 8.00 (d, J=9.5 Hz, 1H), 7.96-7.91 (m, 3H), 7.84 (d, J=8.3 Hz, 2H), 7.43 (d, J=8.6 Hz, 1H), 6.57 (d, J=9.5 Hz, 1H).

### EXAMPLE 18

### 6-[4-(Trifluoromethyl)phenyl]-2(1H)-quinoxalinone

Aqueous hydrogen peroxide solution (30%, 0.200 g, 1.8 mmol) was added dropwise to a stirred slurry of 6-[4-(trifluoromethyl)phenyl]-3,4-dihydro-2(1H)-quinoxalinone (example 13, 0.055 g, 0.188 mmol) in aqueous sodium hydroxide solution (1M, 2 mL, 2.0 mmol) and the mixture heated at 90° C. Dioxane (1 mL) was added to solubilise all the reactants, and heating continued for 0.5 h. The mixture was cooled slightly and acetic acid added dropwise until a solid began to precipitate. Water (30 mL) was added slowly and the mixture stirred 0.5 h at room temperature, then filtered. The solid was washed with water and dried to give the title compound (0.042 g, 76%) as a yellow solid. ¹H NMR (400 MHz, D₆-DMSO) δ 12.54 (br s, 1H), 8.25 (s, 1H), 8.17 (d, J=2.0 Hz, 1H), 8.01-7.97 (m, 3H), 7.84 (d, J=8.3 Hz, 2H), 7.44 (d, J=8.5 Hz, 1H).

### EXAMPLE 19

### 7-[4-(Trifluoromethyl)phenyl]-2H-1,4-benzothiazin-3(4H)-one 1,1-dioxide

A mixture of 7-[4-(trifluoromethyl)phenyl]-2H-1,4-benzothiazin-3(4H)-one

(example 15, 0.178 g, 0.575 mmol) and m-chloroperoxybenzoic acid (0.298 g, 1.73 mmol) in 1,4-dioxane (5.0 mL) was stirred overnight at room temperature. The reaction was then quenched with 20% sodium thiosulfate and 5% sodium bicarbonate and the resulting precipitate was recrystallized from acetonitrile to give the title compound (0.045 g, 23%) as white crystals. MS (ES+) m/e 342 [M+H]⁺.

### EXAMPLE 20

### 3-Oxo-7-[4-(trifluoromethyl)phenyl]-3,4-dihydro-1(2H)-quinoxalinecarbaldehyde

Formic acid (0.200 mL, 5.29 mmol) was added dropwise to stirred acetic anhydride (1.0 mL, 10.6 mmol) and the mixture stirred at 50° C. for 0.5 h. 6-[4-(Trifluoromethyl)phenyl]-3,4-dihydro-2(1H)-quinoxalinone (example 13, 0.055 g, 0.188 mmol) was added and heating continued for 2 h. The mixture was then cooled and the volatiles removed under reduced pressure. The residue was triturated (ether) to give the title compound (0.047 g, 78%) as a solid. ¹H NMR (400 MHz, D₆-DMSO) δ 10.91 (br s, 1H), 8.90 (s, 1H), 7.97 (d, J=8.2 Hz, 2H), 7.90 (d, J=1.9 Hz, 1H), 7.81 (d, J=9.1 Hz, 2H), 7.61 (dd, J=8.3, 1.9 Hz, 1H), 7.13 (d, J=8.3 Hz, 1H), 4.37 (s, 2H).

### EXAMPLE 21

### [3-Oxo-7-[4-(trifluoromethyl)phenyl]-3,4-dihydro-1(2H)-quinoxalinyl]acetonitrile

A mixture of 6-[4-(trifluoromethyl)phenyl]-3,4-dihydro-2(1H)-quinoxalinone (example 13, 0.075 g, 0.257 mmol), bromoacetonitrile (0.037 g, 0.308 mmol), sodium hydrogencarbonate (0.035 g, 0.416 mmol) and dimethylformamide (1.5 mL) was stirred in a microwave reactor at 100° C. for 0.5 h, then cooled. A second portion of bromoacetonitrile (0.050 g, 0.417 mmol) and sodium hydrogencarbonate (0.070 g, 0.833 mmol) was added and the mixture microwaved for 0.25 h at 140° C., then cooled again and partitioned between 0.1 M aqueous HCl and ethyl acetate. The extracts were washed with 0.1 M aqueous HCl, water and brine, then dried (MgSO₄) and the solvent removed under reduced pressure. The residue was chromatographed (silica gel, 50-100% ethyl acetate/hexane) and the product triturated (ether) to give the title compound (0.025 g, 29%) as a solid. ¹H NMR (400 MHz, D₆-DMSO) δ 10.74 (s, 1H), 7.90 (d, J=8.1 Hz, 2H), 7.82 (d, J=8.4 Hz, 2H), 7.27 (d, J=8.1 Hz, 1H), 7.25 (s, 1H), 7.01 (d, J=8.0 Hz, 1H), 4.67 (s, 2H), 3.80 (s, 2H).

### EXAMPLE 22

### Methyl [3-oxo-7-[4-(trifluoromethyl)phenyl]-3,4-dihydro-1(2H)-quinoxalinyl]acetate

The procedure of example 21 was followed here using methyl bromoacetate in place of bromoacetonitrile to give the title compound as a cream solid. ¹H NMR (400 MHz, D₆-DMSO) δ 10.62 (s, 1H), 7.82 (d, J=8.4 Hz, 2H), 7.77 (d, J=8.5 Hz, 2H), 7.09 (dd, J=8.0, 1.8 Hz, 1H), 6.93 (d, J=8.0 Hz, 1H), 6.87 (d, J=1.6 Hz, 1H), 4.34 (s, 2H), 3.92 (s, 2H), 3.69 (s, 3H).

### EXAMPLE 23

### 6-[4-(Trifluoromethyl)phenyl]-3,4-dihydro-1,8-naphthyridin-2(1H)-one

To a stirred solution of 6-bromo-3,4-dihydro-1,8-naphthyridin-2(1H)-one (0.5 g, 2.2 mmol) (J. Med. Chem., 2003, 46(9), 1627-1635.) and [4-(trifluoromethyl)phenyl]boronic acid (0.42 g, 2.2 mmol) in a solution of dimethylformamide (25 mL) and 2M aq. potassium carbonate was added tetrakis(triphenylphosphine)palladium (0) (0.100 g, 0.09 mmol). The reaction was purged with nitrogen then heated to 100° C. and stirred for 18 h. After cooling to room temperature, the reaction was concentrated to dryness under vacuum. Purification by flash chromatography on silica gel (3% methanol/dichloromethane), trituration with (1:1) ether/petroleum ether, filtration and drying under vacuum gave the title compound (470 mg, 73%) as a white solid: ¹H NMR (400 MHz, DMSO-d₆) δ ppm 10.67 (s, 1H), 8.52 (d, J=2.3 Hz, 1H), 8.03 (d, J=2.0 Hz, 1H) 7.93 (d, J=8.3 Hz, 2H), 7.82 (d, J=8.3 Hz, 2H), 2.98 (t, 2H), 2.56 (t, 2H); MS (ES) m/e 293.2 (M+H)⁺.

### EXAMPLE 24

### [3-Oxo-7-[4-(trifluoromethyl)phenyl]-3,4-dihydro-1(2H)-quinoxalinyl]acetic acid

Aqueous sodium hydroxide (1M, 1.0 mL, 1.0 mmol) was added dropwise to an ice-cooled, stirred solution of methyl [3-oxo-7-[4-(trifluoromethyl)phenyl]-3,4-dihydro-1(2H)-quinoxalinyl]acetate (example 22, 0.035 g, 0.096 mmol) in methanol (6 mL) and the mixture stirred while warming to room temperature over 18 h. 1M aqueous hydrochloric acid (2 mL) was added slowly, then the mixture diluted with, water (5 mL). After 0.25 h, the solid was filtered, washed with aqueous methanol and water and dried to give the title compound (0.029 g, 85%) as a cream solid. ¹H NMR (400 MHz, D₆-DMSO) δ 12.85 (br s, 1H), 10.59 (s, 1H), 7.81 (d, J=8.5 Hz, 2H), 7.77 (d, J=8.6 Hz, 2H), 7.08 (dd, J=8.0, 1.8 Hz, 1H), 6.92 (d, J=8.0 Hz, 1H), 6.85 (d, J=1.6 Hz, 1H), 4.20 (s, 2H), 3.94 (s, 2H).

### EXAMPLE 25

### 1-Methyl-6-[4-(trifluoromethyl)phenyl]-3,4-dihydro-2(1H)-quinolinone

A solution of lithium diisopropylamide (344 μL, 2M in THF, 0.69 mmol) in THF (2 mL) was cooled to 0° C. under nitrogen. 6-(4-Trifluoromethylphenyl)-3,4-dihydro-1H-quinolin-2-one (example 12, 100 mg, 0.344 mmol) in THF (2 mL) was added dropwise. The resulting solution was allowed to warm to RT over 1.5 h. The solution was then cooled to −78° C. and methyl iodide (21.2 μL, 0.344 mmol) was added and the resulting solution was stirred from −78° C. to room temperature over 3 h. A mixture of water (30 mL) and ethyl acetate (30 mL) were added and the organic layer was separated and washed with brine (30 mL), dried over MgSO₄, filtered and evaporated under reduced pressure. The crude residue was purified by column:chromatography (3:1, hexane:ethyl acetate) to give the title compound (45 mg, 43%) as an off-white powder. ¹H NMR (400 MHz, D₆-DMSO) δ 7.73 (d, J=8.3 Hz, 2H), 7.61 (d, J=8.3 Hz, 2H), 7.48 (d, J=7.0 Hz, 2H), 7.0 (d, J=9.1 Hz, 1H) 3.11 (s, 3H), 2.78 (dd, J=6.9, 7.9 Hz, 2H), 2.41 (dd, J=7.8, 7.0 Hz, 2H). MS (ES+) m/e 306 [M+H]⁺.

### EXAMPLE 26

### 7-[4-(Trifluoromethyl)phenyl]1,3,4,5-tetrahydro-2H-1-benzazepine-2-one

### a) 7-Bromo-1,3,4,5-tetrahydro-2H-1-benzazepin-2-one

1,3,4,5-tetrahydro-2H-1-benzazepin-2-one (0.22 g, 1.365 mmol) was dissolved in acetic acid (2.0 mL) and sulfuric acid (4 drops). Bromine (0.155 mL, 3.025 mmol) in acetic acid (2.0 mL) was added dropwise at room temperature. The reaction vessel was sealed and stirred at room temperature for 12 hours. The reaction was then poured into ice and neutralized with ammonium hydroxide. The neutral solution was extracted with ethyl acetate and organics combined and washed with saturated sodium bicarbonate and then brine, dried over MgSO₄, filtered and concentrated under reduced pressure. The material was then triturated with ethyl acetate and hexanes to obtain a white crystalline solid. The mother liquor was purified by flash chromotography (100% ethyl acetate) to provide (0.26 g, 79%) of the title compound. ¹H NMR (400 MHz, D₆-DMSO) δ 9.59 (bs, 1H), 7.48 (d, J=2.2 Hz, 1H), 7.40 (dd, J=2.2, 6.1 Hz, 1H), 6.90 (d, J=8.4 Hz, 1H), 2.67 (t, 2H), 2.12 (m, 4H)

### b) 7-[4-(Trifluoromethyl)phenyl]1,3,4,5-tetrahydro-2H-1-benzazepine-2-one

7-Bromo-1,3,4,5-tetrahydro-2H-1-benzazepin-2-one (0.200 g, 0.833 mmol) in dimethylformamide (2.0 mL) was treated with [4-(trifluoromethyl)phenyl]boronic acid (0.28 g, 1.50 mmol), tetrakis(triphenylphosphine)palladium(0) (0.069 g, 0.059 mmol) and 2M potassium carbonate in water (0.80 mL). The reaction was heated to 10° C. for 12 hours and then cooled and twice extracted with ethyl acetate which was then washed with water and then brine. The organic was dried over MgSO₄, filtered and concentrated. Column chromotography (1:1 ethyl acetate:hexane to 10% methanol/dichloromethane) followed by recrystallization from ethanol provided the title compound (0.065 g, 26%) as a solid white material. ¹H NMR (400 MHz, D₆-DMSO) δ 9.67 (s, 1H), 7.84 (m, 4H), 7.66 (s, 1H), 7.61 (d, J=8.1 Hz, 1H), 7.07 (d, J=8.3 Hz, 1H), 2.77 (m, 2H), 2.18 (m, 4H).

### EXAMPLE 27

### 3-(2-Hydroxyethyl)-6-[4-(trifluoromethyl)phenyl]-3,4-dihydro-2(1H)-quinazolinone

### a) 4-Nitro-4′-(trifluoromethyl)-3-biphenylcarbonitrile

A mixture of 4-trifluoromethylbenzeneboronic acid (5.38 g, 28.3 mmol), 5-chloro-2-nitrobenzonitrile (5.11 g, 28 mmol), sodium carbonate (9.0 g, 84 mmol) and tetrakis(triphenylphosphine)palladium (0) (550 mg, 0.48 mmol) in water (20 mL) and dimethylformamide (80 mL) was sealed in pressure bottle under argon and heated at 100° C. for two hours. The mixture was cooled, diluted with water and extracted four times with ethyl acetate, the combined extracts were dried and evaporated onto silica gel. Flash chromatography (silica gel, hexane to 15% ethyl acetate in hexane) gave the title compound (7.0 g, 86%). ¹H NMR (400 MHz, D₆-DMSO) δ 8.62 (d, J=2.1 Hz, 1H), 8.45 (d, J=8.8 Hz, 1H), 8.35 (dd, J=8.9 and 2.3 Hz, 1H), 8.12 (d, J=8.1 Hz, 2H), 7.92 (d, J=8.3 Hz, 2H).

### b) 1-[4-Nitro-4′-(trifluoromethyl)-3-biphenylyl]methanamine

Borane-THF complex (1.0 molar, 100 mL) was added dropwise to a solution of 4-nitro-4′-(trifluoromethyl)-3-biphenylcarbonitrile (9.0 g, 30.8 mmol) in tetrahydrofuran (200 mL) and stirred at ambient temperature overnight in a nitrogen atmosphere. The mixture was then heated at 70° C. for 1.5 hours. The mixture was cooled and carefully treated dropwise with water (10 mL) then 6 molar hydrochloric acid (10 mL) such that gas evolution subsided between drops. The mixture was then heated under reflux for 30 minutes, partially evaporated and basified with ammonia solution. The mixture was diluted with water and extracted three times with ethyl acetate. The combined extracts were dried, evaporated and chromatographed (silica gel, dichloromethane to 10% methanol in dichloromethane) to give the title compound (5.0 g, 55%). ¹H NMR (400 MHz, D₆-DMSO) δ 8.16 (d, J=2.0 Hz, 1H), 8.09 (d, J=8.3 Hz, 1H), 8.02 (d, J=8.7 Hz, 2H), 7.90 (d, J=8.3 Hz, 2H), 7.85 (d, J=8.6 and 2.0 Hz, 1H), 4.05 (s, 2H), 2.04 (br s, 2H).

### c) (2-{[(1,1-Dimethylethyl)(dimethyl)silyl]oxy}ethyl){[4-nitro-4′-(trifluoromethyl)-3-biphenylyl]methyl}amine

A mixture of 1-[4-nitro-4′-(trifluoromethyl)-3-biphenylyl]methanamine (4.16 mg, 1.4 mmol) and (tert-butyldimethylsilyloxy)acetaldehyde was stirred in ethanol (10 mL) for 10 minutes. Sodium triacetoxyborohydride (424 mg, 2.0 mmol) was added and the mixture was stirred for 1 hour. Evaporation and flash chromatography (silica gel, hexane graduating to ethyl acetate) gave the title compound (280 mg, 62%). ¹H NMR (400 MHz, CDCl₃) δ 8.16 (d, J=8.4 Hz, 1H), 7.96 (d, J=2.0 Hz, 1H), 7.76 (s, 4H), 7.69 (dd, J=8.6 and 2.0 Hz, 2H), 4.25 (s, 2H), 3.82 (t, J=4.8 Hz, 2H), 2.96 (t, J=5.0 Hz, 2H), 0.89 (s, 9H), 0.07 (s, 6H).

### d) {[4-Amino-4′-(trifluoromethyl)-3-biphenylyl]methyl}(2-{[(1,1-dimethylethyl)(dimethyl)silyl]oxy}ethyl)amine

A solution of (2-{[(1,1-dimethylethyl)(dimethyl)silyl]oxy}ethyl){[4-nitro-4′-(trifluoromethyl)-3-biphenylyl]methyl}amine (280 mg, 0.62 mmol) in methanol (15 mL) with 10% Palladium on charcoal (100 mg) was stirred in a hydrogen atmosphere for 3 hours. The mixture was filtered through Celite® and the filter bed washed with ethyl acetate. Evaporation gave the title compound (200 mg, 76%). ¹H NMR (400 MHz, CDCl₃) δ 7.63 (m, 4H), 7.40 (dd, J=8.1 and 2.0 Hz, 1H), 7.34 (d, J=2.0 Hz, 1H), 6.77 (d, J=8.3 Hz, 2H), 4.07 (s, 2H), 3.79 (t, J=4.8 Hz, 2H), 2.89 (t, J=4.80 Hz, 2H), 2.05 (br s, 2H), 0.88 (s, 9H), 0.05 (s, 6H).

### e) 3-(2-{[(1,1-Dimethylethyl)(dimethyl)silyl]oxy}ethyl)-6-[4-(trifluoromethyl)phenyl]-3,4-dihydro-2(1H-quinazolinone

A solution of triphosgene (115 mg, 0.39 mmol) in tetrahydrofuran (10 mL) was added dropwise to a mixture of {[4-amino-4′-(trifluoromethyl)-3-biphenylyl]methyl}(2-{[(1,1-dimethylethyl)(dimethyl)silyl]oxy}ethyl)amine (500 mg, 1.18 mmol) and potassium carbonate (1.0 g, 7.2 mmol) in tetrahydrofuran (70 mL) stirred at 0-5° C. over 1 hour. The mixture was then stirred at room temperature overnight. The mixture was poured onto ice, diluted with water and extracted twice with ethyl acetate. The combined extracts were dried and evaporated to give the title compound (480 mg, 90%). ¹H NMR (400 MHz, CDCl₃) δ 7.62 (d, J=8.3 Hz, 2H), 7.56 (d, J=8.3 Hz, 2H), 7.36 (dd, J=8.2 and 2.0 Hz, 1H), 6.93 (s, 1H), 6.71 (d, J=8.2 Hz, 1H), 4.65 (s, 2H), 3.83 (t, J=4.8 Hz, 2H), 3.53 (t, J=5.2 Hz, 2H), 0.84 (s, 9H), 0.00 (s, 6H).

### f) 3-(2-Hydroxyethyl)-6-[4-(trifluoromethyl)phenyl]-3,4-dihydro-2(1H)-quinazolinone

Tetrabutylammonium fluoride (2.0 mL of a 1 molar solution in tetrahydrofuran) was added to a solution of {[4-amino-4′-(trifluoromethyl)-3-biphenylyl]methyl}(2-{[(1,1-dimethylethyl)(dimethyl)silyl]oxy}ethyl)amine (480 mg, 1.06 mmol) in tetrahydrofuran (10 mL) and stirred for 72 hours. The mixture was evaporated and chromatographed (silica gel, 0-10% methanol in dichloromethane) to give the title compound (300 mg, 84%). ¹H NMR (400 MHz, D₆-DMSO) δ 9.36 (s, 1H), 7.83 (d, J=8.4 Hz, 2H), 7.77 (d, J=8.6 Hz, 2H), 7.50-7.56 (m 2H), 6.88 (d, J=8.4 Hz, 1H), 4.78 (t, J=5.8 Hz, 1H), 4.60 (s, 2H), 3.59 (dt, J=5.6 Hz, 2H), 3.39 (t, J=5.2 Hz, 2H).

### EXAMPLE 28

### 6-[4-(Trifluoromethyl)phenyl]-1,2,3,4-tetrahydroquinoline

A solution of lithium aluminum hydride in tetrahydrofuran (1M, 1.70 mL, 1.70 mmol) was injected dropwise into a stirred solution of 6-(4-trifluoromethylphenyl)-3,4-dihydro-1H-quinolin-2-one (example 12, 0.100 g, 0.343 mmol) in tetrahydrofuran (5 mL) at room temperature under nitrogen. After 0.5 h, the solution was cooled in ice and treated sequentially slowly with water (0.065 mL, 3.61 mmol), 3.75M aqueous sodium hydroxide (0.065 mL, 0.244 mmol), and water again (0.200 mL, 11.1 mmol). The mixture was stirred 0.25 h, then sodium sulfate added and the mixture filtered through a plug of sodium sulfate. The cake was washed with ethyl acetate and the solvent removed from the filtrate under reduced pressure. A solution of the residue in methanol was treated with water to precipitate a solid, which was filtered, washed with water and dried to give the title compound (0.073 g, 77%) as a cream solid. ¹H NMR (400 MHz, D₆-DMSO) δ 7.75 (d, J=8.3 Hz, 2H), 7.68 (d, J=8.5 Hz, 2H), 7.30-7.28 (m, 2H), 6.53 (d, J=8.1 Hz, 1H), 6.03 (br s, 1H), 3.23 (m, 2H), 2.74 (t, J=6.3 Hz, 2H), 1.82 (m, 2H).

### EXAMPLE 29

### 1,1-Dimethylethyl 2-oxo-6-[4-(trifluoromethyl)phenyl]-3,4-dihydro-1(2H)-quinolinecarboxylate

A solution of 6-(4-trifluoromethylphenyl)-3,4-dihydro-1H-quinolin-2-one (example 12, 350 mg, 1.20 mmol) in dichloromethane was cooled to 0° C. under nitrogen. 4-Dimethylaminopyridine (20 mg, 0.164 mmol)), triethylamine (185 μL, 1.32 mmol) and di-tert-butyl dicarbonate (288 mg, 1.32 mmol) were added sequentially and the reaction mixture was warmed to room temperature over 16 h. Dichloromethane (10 mL) and 1N HCl (10 mL) were added and the organic layer separated and washed with saturated sodium hydrogen carbonate (20 mL), dried over MgSO₄, filtered and the solvent removed under reduced pressure to yield a yellow crystalline solid (370 mg, 79%). ¹H NMR (400 MHz, D₆-DMSO) δ 7.89 (d, J=8.3 Hz, 2H), 7.81 (d, J=8.6 Hz, 2H), 7.73 (s, 1H), 7.67 (d, J=8.6 Hz, 1H) 6.97 (d, J=8.3 Hz, 1H) 3.04 (t, 2H), 2.66 (t, 2H), 1.55 (s, 9H). MS (ES+) m/e 392 [M+H]⁺.

### EXAMPLE 30

### S-Ethyl 2-oxo-6-[4-(trifluoromethyl)phenyl]-1,2,3,4-tetrahydro-3-quinolinecarbothioate

### a) 1,1-Dimethylethyl 3-[(ethylthio)carbonyl]-2-oxo-6-[4-(trifluoromethyl)phenyl]-3,4-dihydro-1(2H)-quinolinecarboxylate

A solution of 1,1-dimethylethyl 2-oxo-6-[4-(trifluoromethyl)phenyl]-3,4-dihydro-1(2H)-quinolinecarboxylate (example 29, 100 mg, 0.256 mmol) in tetrahydrofuran (4 mL) was cooled to −78° C. under nitrogen. A 2M solution of lithium diisopropylamide (383 μL, 0.766 mmol) in tetrahydrofuran was added dropwise and the resulting solution stirred for 30 minutes. Ethylchlorothioformate (35 mg, 0.281 mmol) was then added and the resulting solution stirred from −78° C. to room temperature over 16 h. A mixture of 1N HCl (5 mL), water (20 mL), and ethyl acetate (20 mL) was added and stirred for 15 minutes. The organic layer was separated, washed with saturated sodium hydrogen carbonate (30 mL) and brine (30 mL), dried over MgSO₄, filtered and solvents removed under reduced pressure. Column chromatography with 0-100% step gradient of ethyl acetate in hexanes gave the desired product as a yellow oil (90 mg, 73%). MS (ES+) m/e 480 [M+H]⁺.

### b) S-Ethyl 2-oxo-6-[4-(trifluoromethyl)phenyl]-1,2,3,4-tetrahydro-3-quinolinecarbothioate

To a solution of 1,1-dimethylethyl 3-[(ethylthio)carbonyl]-2-oxo-6-[4-(trifluoromethyl)phenyl]-3,4-dihydro-1(2H)-quinolinecarboxylate (90 mg, 0.188 mmol) in dichloromethane (2 mL) at room temperature was added a 5% solution of trifluoroacetic acid in dichloromethane (1 mL). After stirring for 30 min at room temperature the solvents were removed by rotary evaporation to yield a white solid (46 mg, 65%). ¹H NMR (400 MHz, D₆-DMSO) δ 10.5 (s, 1H) 7.8 (d, J=8.3 Hz, 2H), 7.7 (d, J=8.6 Hz, 2H), 7.6 (s, 1H), 7.5 (dd, J=8.4, 2.0 Hz, 1H) 6.9 (d, J=8.3 Hz, 1H) 3.9 (t, 1H), 3.2 (d, J=7.1 Hz, 2H) 2.8 (q, 2H), 1.1 (t, J=7.4 Hz, 3H). MS (ES+) m/e 380 [M+H]⁺.

### EXAMPLE 31

### 6-[4-(Trifluoromethyl)phenyl]-1H-4,2,1-benzoxathiazine 2,2-dioxide

### a) 1-Chloro-N-[3-hydroxy-4′-(trifluoromethyl)-4-biphenylyl]methanesulfonamide

Chloromethylsulfonyl chloride (0.23 g, 1.54 mmol) in THF (1.0 mL) was added to 4-amino-3-hydroxy-4′-(trifluoromethyl)biphenyl (example 16c, 0.38 g, 1.5 mmol) in THF (10 mL) and the mixture was stirred for 10 minutes. Pyridine (0.164 g, 2.08 mmol) was added and the reaction was stirred overnight at room temperature. The reaction was acidified to pH=1 with 1N HCl, extracted into ethyl acetate, washed sequentially with water, 5% NaHCO₃, and brine. The organic extracts were dried (Na₂SO₄) and concentrated in vacuo to give a brown solid which was used directly in the next reaction.

### b) 6-[4-(Trifluoromethyl)phenyl]-1H-4,2,1-benzoxathiazine 2,2-dioxide

1-Chloro-N-[3-hydroxy-4′-(trifluoromethyl)-4-biphenylyl]methanesulfonamide from the previous reaction was dissolved in methanol (12 mL) and solid potassium carbonate (0.539 g, 3.9 mmol) was added. The reaction was refluxed for 1.5 hours, cooled to room temperature, acidified to pH=1 with 1N HCl, and concentrated in vacuo. The residue was triturated with water, filtered, and the resulting solid was purified by flash chromatography on silica gel (2%-4% MeOH:CH₂Cl₂) to give the title compound (0.162 g, 2 steps, 33%) as a tan solid. MS (ES+) m/e 328 [M−H]⁺.

### EXAMPLE 32

### 6-[4-(Trifluoromethyl)phenyl]-3,4-dihydro-2(1H)-naphthalenone

6-Bromo-β-tetralone (1.84 g, 8.19 mmol), [4-(trifluoromethyl)phenyl]boronic acid (2.17 g, 11.46 mmol), and tetrakis(triphenylphosphine)palladium (0) (475 mg, 0.41 mmol) in dimethylformamide (12 mL) and 2M aqueous potassium carbonate (12 mL) were heated at 100° C. for 16 h. The reaction was cooled to room temperature, diluted with ethyl acetate (40 mL) and filtered through a pad of Celite®. The organic layer was separated, washed with saturated sodium bicarbonate (40 mL) and brine (40 mL), then dried over MgSO₄, filtered and solvents removed under reduced pressure. The crude residue was purified by column chromatography eluting with hexanes:ethyl acetate gradient to yield yellow crystals (900 mg, 39%). ¹H NMR (400 MHz, D₆-DMSO) δ 7.9 (d, J=8.1 Hz, 2H), 7.84 (d, J=8.2 Hz, 2H), 7.7 (s, 1H), 7.6 (d, J=7.86 Hz, 1H) 7.33 (d, J=7.88 Hz, 1H) 3.69 (s, 2H), 3.15 (t, J=6.6 Hz, 2H) 2.5 (t, 2H). MS (ES+) m/e 291 [M+H]⁺.

### EXAMPLE 33

### 6-[4-(Trifluoromethyl)phenyl]-3,4-dihydro-1H-2,1-benzothiazine 2,2-dioxide

A mixture of 6-bromo-3,4-dihydro-1H-2,1-benzothiazine 2,2-dioxide (B. Loev, M. F. Kormendy, J. Org. Chem., 1965, 30(9), 3163) (0.095 g, 0.362 mmol), 4-(trifluoromethyl)phenylboronic acid (0.138 g, 0.725 mmol), tetrakis(triphenylphosphine)palladium (0) (0.021 g, 0.018 mmol), 2M aqueous potassium acetate (3 mL, 6 mmol) and dioxane (3 mL) was stirred under argon at 90° C. for 18 h, then cooled, poured into 0.1 M aqueous hydrochloric acid (30 mL) and extracted with ethyl acetate. The extracts were washed with water, brine and dried (MgSO₄). The solvent was removed under reduced pressure and the residue azeotroped with toluene three times, then chromatographed (silica gel, 1-3% methanol/dichloromethane). The partially purified product was rechromatographed (silica gel, 20-50% ethyl acetate/hexane) and to give the title compound (0.063 g, 53%) as a solid. ¹H NMR (400 MHz, D₆-DMSO) δ 10.35 (br s, 1H), 7.86 (d, J=8.3 Hz, 2H), 7.79 (d, J=8.4 Hz, 2H), 7.64 (d, J=2.1 Hz, 1H), 7.58 (dd, J=8.4, 2.2 Hz, 1H), 6.89 (d, J=8.4 Hz, 1H), 3.41 (s, 4H).

### EXAMPLE 34

### 7-[4-(Trifluoromethyl)phenyl]-2H-pyrido[3,2-b][1,4]oxazin-3(4H)-one

### a) 7-Bromo-2H-pyrido[3,2-b][1,4]oxazin-3(4H)-one

A mixture of 2H-pyrido[3,2-b][1,4]oxazin-3(4H)-one (1.30 g, 8.66 mmol) and N-bromosuccinimide (1.70 g, 9.53 mmol) in DMF (30 mL) was stirred for three days at room temperature. Water (10 mL) was added and the suspension was cooled with an ice bath. The resulting precipitate was filtered, washed with water, and air-dried in a Buchner funnel to give the title compound (1.37 g, 69%) as a white solid. MS (ES+) m/e 229 [M+H]⁺.

### b) 7-[4-(Trifluoromethyl)phenyl]-2H-pyrido[3,2-b][1,4]oxazin-3(4H)-one

A mixture of 7-bromo-2H-pyrido[3,2-b][1,4]oxazin-3(4H)-one (0.401 g, 1.75 mmol), 4-(trifluoromethyl)benzeneboronic acid (0.497 g, 2.63 mmol), tetrakis(triphenylphosphine)palladium (0) (0.202 g, 0.175 mmol), and 2M aqueous potassium carbonate (4.8 mL, 9.63 mmol) in DMF (11 mL) was heated for 18 hours at 100° C. The reaction was cooled and filtered through Celite®. The filtrate was diluted with ethyl acetate, washed with water and saturated sodium chloride, dried (Na₂SO₄), concentrated in vacuo, and purified by flash chromatography on silica gel (2%-3% MeOH: CH₂Cl₂) to give the title compound (0.032 g, 6%) as a white cotton-like solid. MS (ES+) m/e 295 [M+H]⁺.

### EXAMPLE 35

### 2-[3-Oxo-7-[4-(trifluoromethyl)phenyl]-3,4-dihydro-1(2H)-quinoxalinyl]acetamide

6-[4-(Trifluoromethyl)phenyl]-3,4-dihydro-2(1H)-quinoxalinone (example 13, 0.13 g, 0.44 mmol) was treated with sodium carbonate (0.1 g, 0.94 mmol) and bromoacetamide (0.35 g, 2.54 mmol) in DMF (3.0 mL) in a sealed vessel and microwaved for 15 min. at 180° C. After venting the reaction was partioned between ethyl acetate and water. The organic was washed three times with brine, dried over MgSO₄, filtered and concentrated. Gilson HPLC purification provided the desired product (0.011 g, 3%) as a white powder. ¹H NMR (400 MHz, D₆-DMSO) δ 10.58 (bs, 1H), 7.77 (s, 4H), 7.59 (bs, 1H), 7.18 (bs, 1H), 7.05 (d, J=9.8 Hz, 1H), 6.89 (d, J=8.0 Hz, 1H), 3.98 (s, 2H), 3.93 (s, 2H).

### EXAMPLE 36

### 7-[3-(Trifluoromethyl)phenyl]-2H-1,4-benzothiazin-3(4H)-one

The procedure of example 15 was followed here, using 3-(trifluoromethyl)benzeneboronic acid in place of 4-(trifluoromethyl)benzeneboronic acid, to give the title compound (39%) as a white cotton-like solid. MS (ES+) m/e 310 [M+H]⁺.

### EXAMPLE 37

### 3,3-Dimethyl-6-[4-(trifluoromethyl)phenyl]-3,4-dihydro-2(1H)-quinoxalinone

A mixture of 3-fluoro-4-nitro-4′-(trifluoromethyl)biphenyl (example 13a, 0.100 g, 0.351 mmol), 2-methylalanine (0.109 g, 1.06 mmol), pyridine (0.200 mL, 2.47 mmol), triethylamine (0.150 mL, 1.08 mmol), dioxane (2 mL) and water (0.2 mL) was stirred at 180° C. for 0.5 h in a microwave reactor, then cooled and partitioned between 0.2M aqueous hydrochloric acid (20 mL) and ethyl acetate. The extracts were dried (MgSO₄) and the solvent removed under reduced pressure. A solution of the residue (˜0.100 g) in methanol (5 mL) was stirred with palladium-on-charcoal (5%, 0.09 g, 0.042 mmol) at room temperature under 1 atm of hydrogen for 2 h, then the hydrogen replaced by nitrogen. The mixture was filtered through a plug of filter agent, the solvent removed from the filtrate under reduced pressure and the residue chromatographed (silica gel, 40-100% ethyl acetate/hexane). The product was triturated (ether/hexane) to give the title compound (0.014 g, 13%) as a cream solid. ¹H NMR (400 MHz, D₆-DMSO) δ 10.33 (s, 1H), 7.77 (d, J=9.2 Hz, 2H), 7.76 (d, J=9.1 Hz, 2H), 7.02-6.99 (m, 2H), 6.85 (d, J=7.8 Hz, 1H), 6.17 (s, 1H), 1.26 (s, 6H).

### EXAMPLE 38

### 1,1-Dimethylethyl 3-oxo-7-[4-(trifluoromethyl)phenyl]-2,3-dihydro-4H-1,4-benzothiazine-4-carboxylate

A suspension of 7-[4-(trifluoromethyl)phenyl]-2H-1,4-benzothiazin-3(4H)-one (example 15, 500 mg, 1.61 mmol) in dichloromethane (10 mL) was cooled in an ice bath. 4-Dimethylaminopyridine (30 mg, 0.246 mmol), triethylamine (249 μL, 1.78 mmol), and di-tert-butyl dicarbonate (388 mg, 1.78 mmol) were added sequentially and the resulting solution was stirred from 0° C. to room temperature under nitrogen for 16 h. A mixture of dichloromethane (15 mL) and 1N HCl (15 mL) was then added. The organic layer was separated and washed with saturated sodium hydrogen carbonate (30 mL). After drying over MgSO₄ the solution was filtered and evaporated under reduced pressure to an orange-yellow crystalline solid (500 mg, 85%). MS (ES+) m/e 410 [M+H]⁺.

### EXAMPLE 39

### 6-(4-Fluoro-3-methylphenyl)-3,4-dihydro-1H-quinolin-2-one

A solution of 6-bromo-1,2,3,4-tetrahydro-2-quinolinone (0.149 g, 0.66 mmol), 4-fluoro-3-methylphenylboronic acid (0.123 g, 0.80 mmol), [1,1′-bis(diphenylphosphino)ferrocene]dichloropalladium(II)dichloromethane adduct (0.016 g, 0.02 mmol) and 2M aqueous sodium carbonate solution (1 mL, 2.00 mmol) in N,N-dimethylformamide (2.0 mL) was subjected to microwave irradiation at 120° C. for 10 minutes. The reaction was filtered through a 0.45 μm nylon syringe filter and purified directly by preparative HPLC. The product was allowed to precipitate from the HPLC fractions overnight then was filtered, rinsed with water and vacuum dried to give the product as a white solid. MS (ES) m/e 256 (M+H)⁺.

### EXAMPLE 40

### 4-(2-Hydroxyethyl)-6{4-(trifluoromethyl)phenyl-3,4-dihydro-2(1H)-quinoxalinone

The procedure of example 35 was followed here using 2-bromoethanol in place of bromoacetamide. Column chromotography (5% MeOH/CH₂Cl₂) provided the desired material (0.002 g, 2%) ¹H NMR (400 MHz, MeOD) δ 7.75 (m, 4H), 7.08 (d, J=1.7 Hz, 1H), 7.04 (dd, J=1.7, 6.2 Hz, 1H), 6.92 (d, J=7.9 Hz, 1H), 4.01 (s, 2H), 3.87 (t, 2H), 3.52 (t, 2H).

### EXAMPLE 41

### 4-Methyl-6-[4-(trifluoromethyl)phenyl]3,4-dihydro-2(1H)-quinoxalinone

### a) Ethyl N-methyl-N-[4-nitro-4′-(trifluoromethyl)-3-biphenylyl]glycinate

3-Fluoro-4-nitro-4′-(trifluoromethyl)biphenyl (example 13a, 0.500 g, 1.75 mmol) and sarcosine ethyl ester hydrochloride (0.300 g, 1.95 mmol) in DMF (3.0 mL) were treated with triethylamine (0.48 mL, 3.44 mmol) and heated to 150° C. in the microwave for 15 min. The reaction was diluted with ethyl acetate and washed with 1N HCl and then water, dried over MgSO₄, filtered and concentrated under reduced pressure. Column chromotography (4:1 hexane/ethyl acetate) provided the title compound (0.440 g, 65%) as a solid. ¹H NMR (400 MHz, D₆-DMSO) δ 7.90 (m, 5H), 7.33 (d, J=1.6 Hz, 1H), 7.26 (dd, J=1.7, 6.8 Hz, 1H), 4.19 (s, 2H), 4.13 (q, 2H), 2.90 (s, 3H), 1.18 (t, 3H).

### b) 4-Methyl-6-[4-(trifluoromethyl)phenyl]3,4-dihydro-2(1H)-quinoxalinone

Ethyl N-methyl-N-[4-nitro-4′-(trifluoromethyl)-3-biphenylyl]glycinate (0.370 g, 0.968 mmol) and 10% Pd/C (0.080 g) in methanol (6.0 mL) were allowed to stir under an atmospheric pressure of hydrogen for 1 hour. The reaction was filtered and concentrated under reduced pressure. Crude material was then redissolved in dioxane (3.0 mL) and treated with 4M HCl/dioxane (2.5 mL). The reaction was allowed to stir for 12 hr. and then concentrated under reduced pressure. Column:chromotography (1:1 hexane/ethyl acetate to 100% ethyl acetate) provided the title compound (0.020 g, 7%) as a tan solid. ¹H NMR (400 MHz, CDCl₃) δ 8.15 (bs, 1H), 7.66 (dd, J=8.9, 1.9 Hz, 4H), 7.01 (dd, J=1.7, 6.2 Hz, 1H), 6.87 (s, 1H), 6.82 (d, J=7.9 Hz, 1H), 3.85 (s, 2H), 2.94 (s, 3H).

### EXAMPLE 42

### 6-[4-(Trifluoromethyl)phenyl]-3,4-dihydro-2(1H)-naphthalenone oxime

A solution of 6-[4-(trifluoromethyl)phenyl]-3,4-dihydro-2(1H)-naphthalenone (example 32, 200 mg, 0.689 mmol) in pyridine (5 mL) at room temperature was treated with solid hydroxylamine hydrochloride (120 mg, 1.72 mmol). After 10 min of stirring, pyridine was removed under reduced pressure. Water (30 mL) and ethyl acetate (30 mL) were added. The organic layer was separated and washed with water (15 mL) and brine (30 mL), dried over MgSO₄, filtered and evaporated down to residue to give a brown powder (180 mg, 86%). ¹H NMR (400 MHz, D₆-DMSO) δ 10.57 (s, 0.5H) 10.44 (s, 0.5H) 7.9 (d, J=8 Hz, 2H), 7.8 (d, J=8.4 Hz, 2H), 7.55 (m, 2H), 7.35 (dd, J=7.9 and 3.3 Hz, 1H) 3.73 (s, 1H) 3.52 (s, 1H), 2.9 (m, 2H), 2.55 (t, 1H), 2.47 (t, 1H). MS (ES+) 306 [M+H]⁺.

### EXAMPLE 43

### 8-Fluoro-6-[4-(trifluoromethyl)phenyl]-2(1H)-quinolinone

### a) Ethyl (2E)-3-[4-amino-5-fluoro-4′-(trifluoromethyl)-3-biphenylyl]-2-propenoate

To a stirred solution of 3-bromo-5-fluoro-4′-(trifluoromethyl)-4-biphenylamine (example 11b, 1.0 g, 3.0 mmol), ethyl acrylate (1.0 mL, 9.0 mmol), and diisopropylethylamine (1.1 mL, 6.0 mmol) in propionitrile (20 mL) was added palladium(II)acetate (34 mg, 0.15 mMol) and tri-o-tolylphosphine (0.092 g, 0.30 mmol). The reaction was purged with nitrogen then refluxed at 100° C. for 18 h. After cooling to room temperature, the reaction was evaporated to dryness under vacuum, taken up in 50% ethyl acetate/hexanes, filtered to remove insolubles, and re-evaporated to dryness under vacuum. Purification by flash chromatography on silica gel (20% ethyl acetate/hexanes) gave the title compound (1.02 g, 96%) as a yellowish solid: MS (ES) m/e 354.2 (M+H)⁺.

### b) 8-Fluoro-6-[4-(trifluoromethyl)phenyl]-2(1H)-quinolinone

To a stirred solution of ethyl (2E)-3-[4-amino-5-fluoro-4′-(trifluoromethyl)-3-biphenylyl]-2-propenoate (0.5 g, 1.4 mmol) in methanol (30 mL) was added magnesium turnings (0.18 g, 7.0 mmol). The reaction was stirred at room temperature for 18 h then refluxed for 4 h under nitrogen. The reaction was cooled to room temperature and evaporated to dryness under vacuum. The residue was taken up in ethyl acetate, washed with 1M hydrochloric acid, dried (MgSO₄), filtered and concentrated under vacuum. Purification by flash chromatography on silica gel (30 to 50% ethyl acetate/dichloromethane), trituration with (1:1) ether/petroleum ether, filtration and evaporation to dryness under vacuum gave the title compound (54 mg, 12%) as a white solid: ¹H NMR (400 MHz, DMSO-d₆) δ ppm 11.96 (s, 1H), 8.02 (dd, J=10.0, 1.6 Hz, 1H), 7.99 (s, 1H), 7.98 (d, J=8.4 Hz, 2H), 7.90 (dd, J=12.4, 1.6 Hz, 1H), 7.83 (d, J=8.4 Hz, 2H), 6.64 (d, J=9.6 Hz, 1H); MS (ES) m/e 308.2 (M+H)⁺.

### EXAMPLE 44

### 8-Fluoro-6-[4-(trifluoromethyl)phenyl]-3,4-dihydro-2(1H)-quinolinone

To a stirred solution of ethyl (2E)-3-[4-amino-5-fluoro-4′-(trifluoromethyl)-3-biphenylyl]-2-propenoate (example 43a, 0.5 g, 1.4 mmol) in ethanol (30 mL) was added 10% palladium-on-charcoal (0.100 g). A balloon of hydrogen was attached and the reaction stirred at room temperature for 18 h. The reaction was then filtered through a pad of Celite® and evaporated to dryness under vacuum. The residue was taken up in ethanol (30 mL) and refluxed for 3 days. Purification by flash chromatography on silica gel (30% ethyl acetate/dichloromethane) gave the title compound (94 mg, 22%) as a white solid: ¹H NMR (400 MHz, DMSO-d₆) δ ppm 10.28 (s, 1H), 7.90 (d, J=8.1 Hz, 2H), 7.79 (d, J=8.3 Hz, 2H), 7.54 (dd, J=11.9, 1.8 Hz, 1H), 7.50 (s, 1H), 3.02 (t, 2H), 2.54 (t, 2H); MS (ES) m/e 310.2 (M+H)⁺.

### EXAMPLE 45

### 6-(3,5-Difluoro-phenyl)-3,4-dihydro-1H-quinolin-2-one

The procedure of example 39 was followed here, using 3,5-difluorophenylboronic acid in place of 4-fluoro-3-methylphenylboronic acid, to give the product as a white solid. MS (ES) m/e 260 (M+H)⁺.

### EXAMPLE 46

### 6-(2,2-Difluorobenzo[1,3]dioxol-5-yl)-3,4-dihydro-1H-quinolin-2-one

A solution of 5-bromo-2,2-difluoro-1,3-benzodioxole (0.256 g, 1.08 mmol), bis(pinacolato)diboron (0.302 g, 1.19 mmol), [1,1′-bis(diphenylphosphino)ferrocene]dichloropalladium(II)dichloromethane adduct (0.024 g, 0.03 mmol), and potassium acetate (0.318 g, 3.24 mmol) in N,N-dimethylformamide (2.0 mL) was subjected to microwave irradiation at 100° C. for 6.5 minutes. To this was added more [1,1′-bis(diphenylphosphino)ferrocene]dichloropalladium(II)dichloromethane adduct (0.024 g, 0.03 mmol), 6-bromo-1,2,3,4-tetrahydro-2-quinolinone (0.233 g, 1.03 mmol) in N,N-dimethylformamide (1.0 mL), and 2M aqueous sodium carbonate solution (1.62 mL, 3.24 mmol). The reaction was again subjected to microwave irradiation at 110° C. for 10 minutes then filtered through a 0.45 μm nylon syringe filter and purified directly by preparative HPLC. The product was allowed to precipitate from the HPLC fractions overnight then was filtered, rinsed with water and vacuum dried to give the product as a white solid. MS (ES) m/e 304 (M+H)⁺.

### EXAMPLE 47

### 7-[4-(Trifluoromethyl)phenyl]-1,3,4,5-tetrahydro-2H-1,4-benzodiazepin-2-one

### a) Ethyl N-{[4-amino-4′-(trifluoromethyl)-3-biphenylyl]methyl}glycinate

[3-(Aminomethyl)-4′-(trifluoromethyl)-4-biphenylyl]amine (example 1b, 0.100 g, 0.376 mmol) in DMF (2.0 mL) was treated with ethyl bromoacetate (0.050 mL, 0.453 mmol) and triethylamine (0.10 mL, 0.717 mmol) and heated to 85° C. for 12 hours. The reaction was cooled, diluted with ethyl acetate and washed one time with water, twice with brine, dried over Na₂SO₄, filtered, and concentrated. Column:chromotography (1:1 ethyl acetate/hexane to 100% ethyl acetate) provided the title compound (0.080 g, 60%) as a tan solid. ¹H NMR (400 MHz, CDCl₃) δ 7.62 (s, 4H), 7.37 (dd, J=2.0, 6.0 Hz, 1H), 7.30 (d, J=1.7 Hz, 1H), 6.75 (d, J=8.3 Hz, 1H), 4.84 (bs, 2H), 4.22 (q, 2H), 3.87 (s, 2H), 3.43 (s, 2H), 1.77 (bs, 1H), 1.29 (t, 3H).

### b) 7-[4-(Trifluoromethyl)phenyl]-1,3,4,5-tetrahydro-2H-1,4-benzodiazepin-2-one

Ethyl N-{[4-amino-4′-(trifluoromethyl)-3-biphenylyl]methyl}glycinate (0.080 g, 0.227 mmol) in toluene (4.0 mL) was treated with 1-hydroxybenzotriazole hydrate (0.070 g, 0.5180 mmol) and heated for 72 hours at 85° C. The reaction was diluted with methylene chloride and washed with sat. sodium bicarbonate and water, dried over MgSO₄, filtered and concentrated under reduced pressure. Column chromotography (100% ethyl acetate to 10% methanol/dichloromethane) provided the title compound (0.018 g, 15%) as a white solid. ¹H NMR (400 MHz, D₆-DMSO) δ 9.99 (bs, 1H), 7.89 (d, J=8.2 Hz, 2H), 7.78 (d, J=8.3 Hz, 2H), 7.59 (d, J=8.3 Hz, 1H), 7.56 (s, 1H), 7.20 (d, J=8.3 Hz, 1H), 3.95 (s, 2H), 3.55 (s, 2H).

### EXAMPLE 48

### 6-(3-Fluoro-4-trifluoromethyl phenyl)-3,4-dihydro-1H-quinolin-2-one

The procedure of example 46 was followed here, using 4-bromo-2-fluorobenzotrifluoride in place of 5-bromo-2,2-difluoro-1,3-benzodioxole, to give the title compound as a white solid. MS (ES) m/e 310 (M+H)⁺.

### EXAMPLE 49

### 7-(2,2-Difluorobenzo[1,3]dioxol-5-yl)-1,3,4,5-tetrahydrobenzo[b]azepin-2-one

The procedure of example 46 was followed here, using 7-bromo-1,3,4,5-tetrahydro-2H-1-benzazepin-2-one (example 26a) in place of 6-bromo-1,2,3,4-tetrahydro-2-quinolinone, to give the title compound as a white solid. MS (ES) m/e 318 (M+H)⁺.

### EXAMPLE 50

### 6-(4-Isopropylphenyl)-3,4-dihydro-1H-quinolin-2-one

The procedure of example 39 was followed here, using 4-isopropylbenzeneboronic acid in place of 4-fluoro-3-methylphenylboronic acid, to give the product as a white solid. MS (ES) m/e 266 (M+H)⁺.

### EXAMPLE 51

### 6-(4-Trifluoromethanesulfonylphenyl)-3,4-dihydro-1H-quinolin-2-one

The procedure of example 46 was followed here, using 1-chloro-4-[(trifluoromethyl)sulfonyl]benzene in place of 5-bromo-2,2-difluoro-1,3-benzodioxole, to give the title compound as a light tan solid. MS (ES) m/e 356 (M+H)⁺.

### EXAMPLE 52

### ±6-[4-(Trifluoromethyl)phenyl]-1,2,3,4-tetrahydro-2-naphthalenol

A solution of 6-[4-(trifluoromethyl)phenyl]-3,4-dihydro-2(1 h)-naphthalenone (example 32, 122 mg, 0.421 mmol) in THF (5 mL) at 0° C. was treated with sodium borohydride (5.3 mg, 0.140 mmol). After 1 h of stirring, an additional 10 mg of sodium borohydride was added and the resulting solution stirred for an additional 16 h. 1N HCl (15 mL) and ethyl acetate (15 mL) were added. The organic layer was separated, washed with saturated sodium hydrogen carbonate (30 mL) and brine (30 mL), dried over MgSO₄, filtered and rotary evaporated down to residue. Column chromatography with 0-100% step gradient of ethyl acetate in hexanes gave the desired product as a white powder (50 mg, 41%). ¹H NMR (400 MHz, CDCl₃) δ 7.65 (m, 4H) 7.33 (m, 2H) 7.2 (d, J=7.8 Hz, 1H), 4.2 (m, 1H), 3.25 (dd, 1H), 3.1-2.9 (m, 1H) 2.85-2.65 (m, 2H) 2.1 (m, 1H), 1.9 (m, 1H), 1.65 (bs, 1H). MS (ES+) m/e 585 [2M+H]⁺.

### EXAMPLE 53

### 7-(2,2-Difluorobenzo[1,3]dioxol-5-yl)-4H-benzo[1,4]thiazin-3-one

The procedure of example 46 was followed here, using 7-bromo-2H-1,4-benzothiazin-3(4H)-one in place of 6-bromo-1,2,3,4-tetrahydro-2-quinolinone, to give the title compound as a tan solid. MS (ES) m/e 322 (M+H)⁺.

### EXAMPLE 54

### 3-Methyl-6-[4-(trifluoromethyl)phenyl]-3,4-dihydro-2(1H)-quinazolinone

### a) 4-({[4-(Methoxy)phenyl]methyl}amino)-4′-(trifluoromethyl)-3-biphenylcarbonitrile

A mixture of 4-amino-4′-(trifluoromethyl)-3-biphenylcarbonitrile (example 1a, 3.0 g, 11.44 mmol), 4-methoxybenzyl chloride (1.8 mL, 13.5 mmol) and triethylamine (0.2 mL, 27.85 mmol) in acetonitrile (40 mL) was split into two portions and each subjected to microwave (150° C., 750 s). The portions were recombined, evaporated and flash chromatographed (silica gel, hexane to 30% ethyl acetate in hexane) to give the title compound (3.0 g, 80%). ¹H NMR (400 MHz, D₆-DMSO) δ 7.94 (d, J=2.4 Hz, 1H), 7.82 (d, J=8.4 Hz, 2H), 7.70-7.77 (m, 3H), 7.30 (d, J=8.8 Hz, 2H), 7.15 (t, J=6.2 Hz, 1H), 6.90 (dd, J=6.8 and 1.6 Hz, 2H), 6.74 (d, J=8.8 Hz, 1H), 4.41 (d, J=6.4 Hz, 2H), 3.73 (s, 3H).

### b) [3-(Aminomethyl)-4′-(trifluoromethyl)-4-biphenylyl]{[4-(methoxy)phenyl]methyl}amine

A solution of 4-({[4-(methoxy)phenyl]methyl}amino)-4′-(trifluoromethyl)-3-biphenylcarbonitrile (3.0 g, 7.85 mmol) in anhydrous tetrahydrofuran (100 mL) was added dropwise to lithium aluminum hydride (31 mL of a 1.0 molar solution in hexanes) cooled to −50° C. Cooling was removed and the mixture was stirred at room temperature for 1.5 hours. The mixture was recooled to −50° C. and treated with a mixture of 2.5 molar sodium hydroxide (6.0 mL) and water (17.0 mL) slowly added dropwise. Cooling was removed and ethyl acetate (100 mL) was added. Stirring continued for 30 minutes, the mixture was filtered through Celite®, the filter bed thoroughly washed with ethyl acetate and evaporated to give the title compound (2.7 g, 89%). ¹H NMR (400 MHz, D₆-DMSO) δ 7.77 (d, J=8.0 Hz, 2H), 7.70 (d, J=8.0 Hz, 2H), 7.50 (d, J=2.4 Hz, 1H), 7.42 (dd, J=8.4 and 2.4 Hz, 1H), 7.31 (d, J=8.4 Hz, 2H), 6.89 (d, J=8.4 Hz, 2H), 6.82 (t, J=1.6 Hz, 1H), 6.56 (d, J=8.4 Hz, 1H), 4.33 (d, J=5.6 Hz, 2H), 3.82 (s, 2H), 3.73 (s, 3H), 1.88 (br s, 2H).

### c) 1-{[4-(Methoxy)phenyl]methyl}-6-[4-(trifluoromethyl)phenyl]-3,4-dihydro-2(1/H-quinazolinone

A solution of triphosgene (1.0 g, 3.42 mmol) in tetrahydrofuran (20 mL) was added dropwise to a solution of [3-(aminomethyl)-4′-(trifluoromethyl)-4-biphenylyl]{[4-(methoxy)phenyl]methyl}amine (2.7 g, 7.03 mmol). The mixture was stirred for six hours, evaporated and chromatographed (silica gel, hexane to 30% ethyl acetate in hexane) to give the title compound (800 mg, 27%). ¹H NMR (400 MHz, CDCl₃) δ 7.67 (d, J=8.3 Hz, 2H), 7.61 (d, J=8.3 Hz, 2H), 7.50 (dd, J=8.5 and 2.4 Hz, 1H), 7.24-7.31 (m, 4H), 6.87-6.90 (m, 3H), 5.13 (s, 2H), 4.63 (s, 2H), 3.80 (s, 3H).

### d) 3-Methyl-1-{[4-(methoxy)phenyl]methyl}-6-[4-(trifluoromethyl)phenyl]-3,4-dihydro-2(1H)-quinazolinone

1-{[4-(Methoxy)phenyl]methyl}-6-[4-(trifluoromethyl)phenyl]-3,4-dihydro-2(1H)-quinazolinone (200 mg, 0.48 mmol) was added to a suspension of sodium hydride (60% in oil, 80 mg, 2.0 mmol) in dimethylformamide (10 mL) and stirred for 15 minutes under nitrogen. Methyl iodide (0.5 ml, 8.0 mmol) was added and the mixture was stirred for 3 hours. the mixture was poured onto ice, the solid collected, washed with water and hexane, then dried under vacuum to give the title compound (200 mg, 98%). ¹H NMR (400 MHz, D₆-DMSO) δ 7.82 (d, J=8.4 Hz, 2H), 7.76 (d, J=8.4 Hz, 2H), 7.56 (d, J=2.0 Hz, 1H), 7.52 (dd, J=8.4 and 2.0 Hz, 1H), 7.19 (d, J=8.8 Hz, 2H), 6.65-6.90 (m, 3H), 5.05 (s, 2H), 4.55 (s, 2H), 3.71 (s, 3H).

### e) 3-Methyl-6-[4-(trifluoromethyl)phenyl]-3,4-dihydro-2(1H-quinazolinone

Trifluoroacetic acid (2.0 mL) was added to 3-methyl-1-{[4-(methyloxy)phenyl]methyl}-6-[4-(trifluoromethyl)phenyl]-3,4-dihydro-2(1H)-quinazolinone (190 mg, 0.44 mmol) and was stirred for 10 minutes under gentle warming. The mixture was carefully transferred onto ice and diluted with water. The mixture was then extracted twice with ethyl acetate and the combined organic solutions washed with 50% brine, sodium hydrogen carbonate solution, dried and evaporated to a solid which recrystallised from ethyl acetate to give the title compound (60 mg, 45%). ¹H NMR (400 MHz, D₆-DMSO) δ 9.41 (s, 1H), 7.83 (d, J=8.3 Hz, 2H), 7.77 (d, J=8.4 Hz, 2H), 7.50-7.56 (m, 2H), 6.88 (d, J=8.3 Hz, 1H), 4.48 (s, 2H), 2.89 (s, 3H).

### EXAMPLE 55

### 8-[4-(Trifluoromethyl)phenyl]-2,3-dihydro-1,5-benzothiazepin-4(5H)-one

### a) Methyl 3-{[4-nitro-4′-(trifluoromethyl)-3-biphenylyl]thio}propanoate

A mixture of 3-fluoro-4-nitro-4′-(trifluoromethyl)biphenyl (example 13a, 1.2 g, 4.19 mmol), methyl 3-mercaptopropionate (0.504 g, 4.19 mmol), and solid potassium carbonate (0.579 g, 4.19 mmol) in dimethylformamide (5.0 mL) was stirred overnight at room temperature. The reaction was diluted with water and the resulting precipitate was washed with water and dried to give the title compound (1.52 g, 94%) as a yellow powder. MS (ES+) m/e 386 [M+H]⁺.

### b) Methyl 3-{[4-amino-4′-(trifluoromethyl)-3-biphenylyl]thio}propanoate

Zinc powder (2.53 g, 38.7 mmol) was added to a solution of methyl 3-{[4-nitro-4′-(trifluoromethyl)-3-biphenylyl]thio}propanoate (1.49 g, 3.87 mmol) in acetic acid (35 mL) and the reaction was stirred for 3.5 hours at room temperature. The reaction was filtered through Celite® and concentrated in vacuo to give the title compound (1.37 g, 99%) as a crystalline orange solid. MS (ES+) m/e 356 [M+H]⁺.

### c) 8-[4-(Trifluoromethyl)phenyl]-2,3-dihydro-1,5-benzothiazepin-4(5H)-one

Methyl 3-{[4-amino-4′-(trifluoromethyl)-3-biphenylyl]thio}propanoate (1.37 g, 3.87 mmol) was dissolved in xylenes (25 mL) and the mixture was refluxed for 18 hours. The reaction was cooled to room temperature and the resulting precipitate was filtered and dried to give the title compound (0.871 g, 69%) as light tan crystals. MS (ES+) m/e 324 [M+H]⁺.

### EXAMPLE 56

### 7-(4-Trifluoromethanesulfonylphenyl)-4H-benzo[1,4]thiazin-3-one

The procedure of example 46 was followed here, using 7-bromo-2H-1,4-benzothiazin-3(4H)-one in place of 6-bromo-1,2,3,4-tetrahydro-2-quinolinone, and 1-chloro-4-[(trifluoromethyl)sulfonyl]benzene in place of 5-bromo-2,2-difluoro-1,3-benzodioxole, to give the title compound as a cream solid. MS (ES) m/e 374 (M+H)⁺.

### EXAMPLE 57

### 7-(3-Fluoro-4-trifluoromethylphenyl)-4H-benzo[1,4]thiazin-3-one

The procedure of example 46 was followed here, using 7-bromo-2H-1,4-benzothiazin-3(4H)-one in place of 6-bromo-1,2,3,4-tetrahydro-2-quinolinone, and 4-bromo-2-fluoro-1-(trifluoromethyl)benzene in place of 5-bromo-2,2-difluoro-1,3-benzodioxole, to give the title compound as a cream solid. MS (ES) m/e 328 (M+H)⁺.

### EXAMPLE 58

### 8-Fluoro-6-[3-fluoro-4-(trifluoromethyl)phenyl]-3,4-dihydro-2(1H)-quinolinone

### a) 3,3′-Difluoro-4′-(trifluoromethyl)-4-biphenylamine

To a stirred solution of 2-fluoro-4-iodoaniline (7.0 g, 29.5 mmol), bis(pinacolato)diboron (9.8 g, 38.6 mmol) and potassium acetate (8.7 g, 88.6 mmol) in dimethylformamide (150 mL) was added [1,1′-bis(diphenylphosphino)ferrocene]dichloropalladium(II)dichloromethane adduct (0.66 g, 0.9 mmol). After purging with nitrogen, the reaction was heated to 80° C. and stirred for 4 h. 4-Bromo-2-fluoro-1-(trifluoromethyl)benzene (7.2 g, 29.6 mmol), potassium carbonate (10.2 g, 73.8 mmol), water (20 mL) and [1,1′-bis(diphenylphosphino)ferrocene]dichloropalladium(II)dichloromethane adduct (0.66 g, 0.9 mMol) were added and the reaction stirred at 100° C. for 18 h. After cooling to room temperature, the reaction was evaporated to dryness under vacuum. The residue was taken up in ethyl acetate, filtered to remove insolubles, washed with water, brine, dried (Na₂SO₄), filtered and concentrated under vacuum. Purification by flash chromatography on silica gel (20% ethyl acetate/hexanes), trituration with hexane, filtration and drying under vacuum gave the title compound (4.93 g, 61%) as a white solid: MS (ES) m/e 274.0 (M+H)⁺.

### b) 3-Bromo-3′,5-difluoro-4′-(trifluoromethyl)-4-biphenylamine

To a stirred solution of 3,3′-difluoro-4′-(trifluoromethyl)-4-biphenylamine (6.0 g, 22 mmol) in acetic acid (100 mL) was added dropwise bromine (1.25 mL, 24 mmol). The reaction was heated to 40° C. and stirred for 4 h. After cooling to room temperature, the reaction was evaporated to dryness under vacuum, taken up in ethyl acetate, washed with 1M aq sodium carbonate, brine, dried (Na₂SO₄), filtered and evaporated under vacuum. Trituration with hexane, filtration and drying under vacuum gave the title compound (6.12 g, 79%) as a beige solid: MS (ES) m/e 351.8 (M+H)⁺.

### c) Ethyl (2E)-3-[4-amino-3′,5-difluoro-4′-(trifluoromethyl)-3-biphenylyl]-2-propenoate

To a stirred solution of 3-bromo-3′,5-difluoro-4′-(trifluoromethyl)-4-biphenylamine (6.1 g, 17.3 mmol), ethyl acrylate (5.6 mL, 52 mMol) and diisopropylethylamine (6.1 mL, 35 mmol) in propionitrile (100 mL) was added palladium(II)acetate (195 mg, 0.87 mmol) and tri-o-tolylphosphine (528 mg, 1.7 mMol). The reaction was purged with nitrogen, heated to 100° C. and stirred for 18 h. After cooling to room temperature, the reaction was evaporated to dryness under vacuum, taken up in 50% ethyl acetate/hexane, filtered to remove insolubles, and concentrated under vacuum. Purification by flash chromatography on silica gel (20% ethyl acetate/hexane) gave the title compound (6.05 g, 94%) as a yellow solid: MS (ES) m/e 372.0 (M+H)⁺.

### d) 8-Fluoro-6-[3-fluoro-4-(trifluoromethyl)phenyl]-3,4-dihydro-2(1H-quinolinone

To a stirred solution of ethyl (2E)-3-[4-amino-3′,5-difluoro-4′-(trifluoromethyl)-3-biphenylyl]-2-propenoate (6.05 g, 16.3 mmol) in ethanol (50 mL) was added 10% palladium-on-charcoal (0.5 g). A balloon of hydrogen was attached and the reaction was stirred for 18 h. The reaction was filtered through a pad of Celite®, rinsed with ethanol and evaporated to dryness under vacuum. The solid which remained was dissolved in acetic acid (50 ml), heated to 80° C., and stirred for 4 h. After cooling to room temperature, the reaction was evaporated to dryness, triturated with 20% ethyl acetate/hexane, filtered and dried under vacuum to give the title compound (4.29 g, 82%) as a white solid: ¹H NMR (400 MHz, DMSO-d₆) δ ppm 10.32 (s, 1H), 7.88 (d, J=12.6 Hz, 1H), 7.82 (t, 1H), 7.75 (dd, 1H), 7.63 (dd, J=11.9, 2.0 Hz, 1H), 7.57 (s, 1H), 3.02 (t, 2H), 2.54 (t, 2H); MS (ES) m/e 328.2 (M+H)⁺.

### EXAMPLE 59

### 6-[3-Nitro-4-(trifluoromethyl)phenyl]-3,4-dihydro-2(1H)-quinolinone

### a) (2-Oxo-1,2,3,4-tetrahydro-6-quinolinyl)boronic acid

To a solution of 6-bromo-3,4-dihydro-2(1H)-quinolinone (1 g, 4.4 mmol) in dry tetrahydrofuran (15 mL) at 0° C. was added sodium hydride (212 mg, 60% dispersion, 5.23 mmol). After the mixture was stirred for half an hour at 0° C., it was cooled to −78° C. and n-butyllithium (1.6 M in hexane, 3.1 mL, 4.96 mmol) was added. The mixture was kept stirring for another half hour, then trimethyl borate (1.5 mL, 13.2 mmol) was added. The reaction was warmed to the room temperature, quenched with ice water and extracted with ethyl acetate (3×30 mL). The combined extracts was dried over MgSO₄, filtered and concentrated in vacuo. The residue was used in the next step without purification.

### b) 4-Bromo-2-nitro-1-(trifluoromethyl)benzene

To a solution of 1,4-dibromo-2-nitrobenzene (5 g, 17.8 mmol) in N-methylpyrrolidinone (40 mL) were added methyl difluoro(fluorosulfonyl)acetate (4.5 mL, 35.6 mmol) and copper(I)iodide. The mixture was heated at 80° C. overnight, decolorized with activated charcoal, diluted with brine and extracted with ethyl acetate (3×30 mL). The combined extracts was dried over MgSO₄, concentrated in vacuo and purified by flash chromatography (0-100% ethyl acetate in hexanes) to give the title compound (4.1 g, 85%) as a yellow oil. ¹H NMR (400 MHz, CDCl₃) δ 8.15 (s, 1H), 7.90 (d, J=8.1 Hz, 1H), 7.75 (d, J=8.1 Hz, 1H).

### c) 6-[3-Nitro-4-(trifluoromethyl)phenyl]-3,4-dihydro-2(1H)-quinolinone

To a solution of (2-oxo-1,2,3,4-tetrahydro-6-quinolinyl)boronic acid (720 mg, 3.79 mmol) in dioxane (10 mL) and aqueous potassium carbonate (2 M, 10 mL) were added tetrakis(triphenylphosphine)palladium (0) (219 mg, 0.19 mmol) and 4-bromo-2-nitro-1-(trifluoromethyl)benzene (1.0 g, 3.79 mmol). The mixture was heated at 90° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (764 mg, 60%) as a yellow solid. ¹H NMR (400 MHz, CDCl₃) δ 8.27 (br s, 1H), 8.07 (s, 1H), 7.89 (d, J=1.0 Hz 1H), 7.49 (m, 2H), 6.93 (d, J=8.8 Hz, 1H), 3.31 (t, J=7.6 Hz, 2H), 2.74 (t, J=7.6 Hz, 2H). MS (ES+) m/e 337 [M+H]⁺.

### EXAMPLE 60

### 6-[3-Chloro-4-(trifluoromethyl)phenyl]-3,4-dihydro-2(1H)-quinolinone

To a solution of [3-chloro-4-(trifluoromethyl)phenyl]boronic acid (100 mg, 0.45 mmol) in dioxane (5 mL) and aqueous potassium carbonate (2 M, 5 mL) were added tetrakis(triphenylphosphine)palladium (0) (26 mg, 0.022 mmol) and 6-bromo-3,4-dihydro-2(1H)-quinolinone (100 mg, 0.45 mmol). The mixture was heated at 90° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (55 mg, 38%) as a white solid. ¹H NMR (400 MHz, CDCl₃) δ 8.58 (br s, 1H), 7.86 (d, J=2.3 Hz, 1H), 7.66 (dd, J=2.1, 8.4 Hz, 1H), 7.57 (d, J=8.3 Hz, 1H), 7.42 (m, 2H), 6.92 (d, J=8.6 Hz, 1H), 3.09 (t, J=7.5 Hz, 2H), 2.73 (t, J=7.6 Hz, 2H). MS (ES+) m/e 326 [M+H]⁺.

### EXAMPLE 61

### 6-[6-(Trifluoromethyl)-3-pyridinyl]-3,4-dihydro-2(1H)-quinolinone

To a solution of (2-oxo-1,2,3,4-tetrahydro-6-quinolinyl)boronic acid (example 59a, 21 mg, 0.11 mmol) in dioxane (5 mL) and aqueous potassium carbonate (2 M, 5 mL) were added tetrakis(triphenylphosphine)palladium (0) (6.4 mg, 0.05 mmol) and 5-bromo-2-(trifluoromethyl)pyridine (30 mg, 0.11 mmol). The mixture was heated at 90° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (27 mg, 84%) as a white solid. ¹H NMR (400 MHz, CDCl₃) δ 8.84 (d, J=2.3 Hz, 1H), 8.61 (br s, 1H), 7.93 (dd, J=1.8, 8.1 Hz, 1H), 7.67 (d, J=8.1 Hz, 1H), 7.38 (m, 2H), 6.89 (d, J=7.0 Hz, 1H), 3.02 (t, J=7.1 Hz, 2H), 2.65 (t, J=7.8 Hz, 2H). MS (ES+) m/e 293 [M+H]⁺.

### EXAMPLE 62

### N,N-Dimethyl-4-(2-oxo-1,2,3,4,-tetrahydro-6-quinolinyl)benzenesulfonamide

Following the procedure outlined in Example 65 except substituting (4-(dihydroxyboranepinacol ester)phenyldimethylsulfonamide for 4-isopropylthiophenyl boronic acid, the title compound was prepared. MS (ES) m/e 331 (M+H)⁺.

### EXAMPLE 63

### 6-[4-(Dimethylamino)phenyl]-3,4-dihydro-2(1H)-quinolinone

Following the procedure outlined in Example 65 except substituting 4-dimethylaminophenyl boronic acid for 4-isopropyl thiophenyl boronic acid, the title compound was prepared. MS (ES) m/e 267 (M+H)⁺.

### EXAMPLE 64

### 6-(4-tert-Butylphenyl)-3,4-dihydro-1H-quinolin-2-one

The procedure of example 39 was followed here, using 4-tert-butylbenzeneboronic acid in place of 4-fluoro-3-methylphenylboronic acid, to give the product as tan platelets. MS (ES) m/e 280 (M+H)⁺.

### EXAMPLE 65

### 6-{4-[(1-Methylethyl)thio]phenyl}-3,4-dihydro-2(1H)-quinolinone

6-Bromo-3,4-dihydro-1H-quinolin-2-one (0.500 g, 2.0 mmol), and 4-isopropyl thiophenyl boronic acid (0.470 g, 2.4 mmol) were dissolved in DMF (6 mL). To this solution was added 2M aq. sodium carbonate (3 mL, 6.0 mmol) followed by [1,1′-bis(diphenylphosphino)ferrocene]dichloropalladium(II)dichloromethane complex (0.015 g, 0.02 mmol). The reaction was heated in the Emrys Optimizer microwave reactor at 110° C. for 10 min. The reaction was filtered through Celite®e and purified by reverse phase HPLC (20-95% CH₃CN:H₂O, 0.1% TFA) to give the desired material (170 mg, 29%) as the trifluoroacetate salt. MS (ES) m/e 297 (M+H)⁺.

### EXAMPLE 66

### 6-[4-(Trifluoromethyl)phenyl]chroman-2-one

### a) 6-Bromochroman-2-one

To a solution of 3,4-dihydrocoumarin (3 mL, 24 mmol) in chloroform (15 mL) was added a solution of bromine (1.23 mL, 24 mmol) in acetic acid (5 mL). The resulting dark orange solution was stirred at room temperature for 16 h and turned near colorless. The reaction mixture was diluted with dichloromethane (40 mL) and washed with saturated sodium hydrogen carbonate (40 mL) followed by water (40 mL). The organic layer was dried over MgSO₄, filtered and solvent removed under reduced pressure to give a crude white powder (4 gm) containing the desired mono brominated product and a bis brominated product in a 70:30 ratio. Purification of 1 gm of crude residue by rp-HPLC yielded the desired product as a white powder (470 mg). ¹H NMR (400 MHz, D₆-DMSO) δ 7.61 (s, 1H) 7.51 (dd, 1H) 7.1 (d, J=8.6 Hz, 1H), 3.1 (t, 2H), 2.7 (t, 2H).

### b) 3-[4-Hydroxy-4′-(trifluoromethyl)biphenyl-3-yl]propanoic acid

A mixture of 6-bromochroman-2-one (1.5 g, 6.64 mmol), [4-(trifluoromethyl)phenyl]boronic acid (1.87 g, 9.95 mmol), tetrakis(triphenylphosphine)palladium (0) (384 mg, 0.33 mmol) and potassium carbonate (2.75 g, 19.90 mmol) in dioxane (20 mL) was heated at 110° C. under argon for 16 h. The reaction mixture was cooled to room temperature, diluted with ethyl acetate (50 mL), filtered through Celite® and solvents removed under reduced pressure. The crude residue was partitioned between methanol (50 mL), 1N HCl (50 mL) and dichloromethane (75 mL). The organic layer was separated, dried over MgSO₄, and evaporated to dryness. The residue was dissolved in acetonitrile (4 mL) and purified by rp-HPLC to yield the title compound a solid white powder (500 mg, 24%). ¹H NMR (400 MHz, DMSO-d₆) δ 12.11 (s, 1H) 9.76 (s, 1H) 7.73-7.81 (m, 4H) 7.49 (d, J=2.27 Hz, 1H) 7.43 (dd, J=8.34, 2.53 Hz, 1H) 6.91 (d, J=8.34 Hz, 1H) 2.83 (t, J=7.71 Hz, 2H) 2.55 (d, J=7.83 Hz, 2H). MS (ES+) m/e 310 [M+H]⁺.

### c) 6-[4-(Trifluoromethyl)phenyl]chroman-2-one

3-[4-Hydroxy-4′-(trifluoromethyl)biphenyl-3-yl]propanoic acid (500 mg) in dichloromethane (10 mL) was treated with 10% trifluoroacetic acid in dichloromethane (10 mL) and allowed to stir at room temperature for 15 min. The solvent was removed under reduced pressure and the crude residue dissolved in DMSO (4 mL) and purified by rp-HPLC to yield the desired product as a pale brown powder (25 mg, 5%). ¹H NMR (400 MHz, DMSO-d₆) δ 7.85-7.92 (m, 2H) 7.78-7.84 (m, 2H) 7.72 (s, 1H) 7.66 (dd, J=8.34, 2.02 Hz, 1H) 7.19 (d, J=8.34 Hz, 1H) 3.09 (t, J=7.33 Hz, 2H) 2.85 (t, J=7.20 Hz, 2H). MS (ES+) m/e 293 [M+H]⁺.

### EXAMPLE 67

### 7-[4-(Trifluoromethyl)phenyl]-1,3,4,5-tetrahydro-2H-1,3-benzodiazepin-2-one

### a) [(2-Nitrophenyl)methyl]amine

To a cold (0° C.) solution of (2-nitrophenyl)acetonitrile (3.0 g, 18.5 mmol) in dry THF (100 mL) was added borane-THF (92.6 mL of a 1.0M solution in THF, 92.6 mmol). After 5 min, the reaction mixture was allowed to warm to room temperature, stirred for 5 h, re-cooled to 0° C., quenched carefully by dropwise addition of 6N HCl (155 mL, 925 mmol) and allowed to stand overnight at room temperature. A majority of the THF was removed under reduced pressure and the aqueous layer was adjusted to pH 8-9 with 6N NaOH. The aqueous layer was extracted with five 50 mL portions of ethyl acetate and the combined extracts were dried over sodium sulfate, filtered and concentrated under reduced pressure. The residue was purified by reverse phase HPLC (MeCN/H₂O/TFA) to provide 0.70 g (23%, orange solid) of [(2-nitrophenyl)methyl]amine as the free base after neutralization. ESMS [M+H]⁺: 167.2.

### b) 2-(Aminomethyl)aniline

A solution of [(2-nitrophenyl)methyl]amine (170 mg, 1.02 mmol) in methanol (10 mL) was purged with argon for 5 min, then 10% palladium on carbon (−20 mg) was added. The flask was equipped with a hydrogen balloon and the reaction mixture was stirred vigorously for 18 h, purged with argon and filtered through a pad of Celite®. The filtrate was concentrated under reduced pressure to provide 139 mg of 2-(aminomethyl)aniline which was used in the subsequent reaction without purification.

### c) 1,3,4,5-Tetrahydro-2H-1,3-benzodiazepin-2-one

To a solution of 2-(aminomethyl)aniline (139 mg, 1.02 mmol) in dry acetonitrile (12 mL) was added 1,1′-carbonyldiimidazole (165 mg, 1.02 mmol). The resultant solution was heated to 70° C. for 3.5 h and concentrated under reduced pressure. The residue was dissolved in dichloromethane and washed with water. The aqueous layer was back extracted with dichloromethane and the combined extracts were dried over sodium sulfate, filtered and concentrated under reduced pressure. The residue was purified by silica gel chromatography (7% methanol in dichloromethane) to give 61 mg (36%, 2 steps) of 1,3,4,5-tetrahydro-2H-1,3-benzodiazepin-2-one as an off-white solid. ESMS [M+H]⁺: 163.0.

### d) 7-Bromo-1,3,4,5-tetrahydro-2H-1,3-benzodiazepin-2-one

To a solution of 1,3,4,5-tetrahydro-2H-1,3-benzodiazepin-2-one (55 mg, 0.340 mmol) in dry dimethylformamide (3 mL) was added N-bromosuccinimide (67 mg, 0.374 mmol) in one portion. After 30 min, the reaction mixture was quenched with water (5 mL) and extracted with three 10 mL portions of ethyl acetate. The combined extracts were dried over sodium sulfate, filtered and concentrated under reduced pressure. The residue was purified by silica gel chromatography (7% methanol in dichloromethane) to give 55 mg (67%,) of 7-bromo-1,3,4,5-tetrahydro-2H-1,3-benzodiazepin-2-one as a white solid. ESMS [M+H]⁺: 241.0.

### e) 7-[4-(Trifluoromethyl)phenyl]-1,3,4,5-tetrahydro-2H-1,3-benzodiazepin-2-one

To a solution of 7-bromo-1,3,4,5-tetrahydro-2H-1,3-benzodiazepin-2-one (52 mg, 0.216 mmol) in DMF (1.1 mL) was added 4-(trifluoromethylphenyl)boronic acid (49 mg, 0.259 mmol), 2.0M aqueous potassium carbonate (0.27 mL, 0.54 mmol) followed by tetrakis(triphenylphosphine)palladium (0) (12.5 mg, 0.011 mmol). The mixture was heated to 80° C. with stirring under argon. After 2 h, the reaction was allowed to cool to room temperature, diluted with water (3 mL) and extracted with ethyl acetate (3×10 mL) followed by dichloromethane (2×10 mL). The combined extracts were dried over sodium sulfate, filtered and concentrated under reduced pressure. The residue was purified by silica gel chromatography (7% methanol in dichloromethane) followed by reverse phase HPLC to give 30 mg (45%) of the title compound as a white solid. ESMS [M+H]⁺: 307.2. ¹H NMR (400 MHz, dmso-d₆): δ 8.81 (s, 1H), 7.84 (d, J=8.4 Hz, 2H), 7.76 (d, J=8.4 Hz, 2H), 7.51-7.48 (m, 2H), 7.14 (d, J=8.4 Hz, 1H), 7.13 (s, 1H), 3.26 (m, 2H), 3.00 (m, 2H).

### EXAMPLE 68

### 6-[3-Amino-4-(trifluoromethyl)phenyl]-3,4-dihydro-2(1H)-quinolinone

To the solution of 6-[3-nitro-4-(trifluoromethyl)phenyl]-3,4-dihydro-2(1H)-quinolinone (example 59, 100 mg, 0.30 mmol) in acetic acid (10 mL) was added zinc dust (196 mg, 3 mmol). The mixture was stirred overnight at room temperature and filtered. The filtrate was concentrated in vacuo and purified by flash chromatography (0-100% ethyl acetate in hexanes) to give the title compound (80 mg, 88%) as a white solid. ¹H NMR (400 MHz, CD₃OD) δ 7.5 (m, 4H), 7.1 (s, 1H), 6.9 (d, J=7.0 Hz, 1H), 3.05 (t, J=7.1 Hz, 2H), 2.6 (t, J=7.1 Hz, 2H). MS (ES+) m/e 307 [M+H]⁺.

### EXAMPLE 69

### 8-Amino-6-[4-(trifluoromethyl)phenyl]-3,4-dihydro-2(1H)-quinolinone

To a stirred solution of 8-nitro-6-[4-(trifluoromethyl)phenyl]-3,4-dihydro-2(1H)-quinolinone (example 77, 0.55 g, 1.6 mmol) in ethanol (30 mL) was added 10% palladium-on-charcoal (0.25 g). A balloon of hydrogen was attached and the reaction stirred at room temperature for 18 h. The reaction was filtered through a pad of Celite®, rinsed with ethanol, and evaporated to dryness under vacuum. Purification by flash chromatography on silica gel (2-5% methanol/dichloromethane) gave the title compound (50 mg, 9%) as a white solid. ¹H NMR (400 MHz, DMSO-d₆) δ ppm 9.46 (s, 1H), 7.75 (s, 4H), 6.92 (d, J=2.0 Hz, 1H), 6.83 (d, J=1.5 Hz, 1H), 5.24 (s, 2H), 2.88 (t, 2H), 2.45 (t, 2H). MS (ES) m/e 307.2 (M+H)⁺.

### EXAMPLE 70

### 7-(4-Trifluoromethanesulfonylphenyl)-1,3,4,5-tetrahydrobenzo[b]azepin-2-one

The procedure of example 46 was followed here, using 7-bromo-1,3,4,5-tetrahydro-2H-1-benzazepin-2-one (example 26a) in place of 6-bromo-1,2,3,4-tetrahydro-2-quinolinone, and 1-chloro-4-[(trifluoromethyl)sulfonyl]benzene in place of 5-bromo-2,2-difluoro-1,3-benzodioxole, to give the title compound as fine, colorless needles. MS (ES) m/e 370 (M+H)⁺.

### EXAMPLE 71

### 6-[4-(Methylsulfonyl)phenyl]-3,4-dihydro-2(1H)-quinolinone

Following the procedure outlined in Example 65 except substituting [4-(methylsulfonyl)phenyl]boronic acid for 4-isopropylthiophenyl boronic acid, the title compound was prepared. MS (ES) m/e 302 (M+H)⁺.

### EXAMPLE 72

### 6-[4-Nitrophenyl)-3,4-dihydro-2(1H)-quinolinone

Following the procedure outlined in Example 65 except substituting (4-nitrophenyl)boronic acid for 4-isopropylthiophenyl boronic acid, the title compound was prepared. MS (ES) m/e 269 (M+H)⁺.

### EXAMPLE 73

### 4-Amino-6-[4-(trifluoromethyl)phenyl]-2(1H)-quinolinone

### a) 4-[(Phenylmethyl)amino]-6-[4-(trifluoromethyl)phenyl]-2(1H)-quinolinone

A mixture of 6-bromo-4-hydroxy-2(1H)-quinolinone (D. R. Buckle et. al., J. Med. Chem., 1975, 18(7), 726) (0.244 g, 1.02 mmol) and benzylamine (5 mL) was heated under reflux under nitrogen for 18 h, then cooled, diluted with 1M aqueous hydrochloric acid and extracted with ethyl acetate. The extracts were washed with 1M aqueous hydrochloric acid, water, and brine, dried (MgSO₄) and the solvent removed under reduced pressure. The residue was chromatographed (silica gel, 3-10% methanol/dichloromethane) to give an impure sample of 6-bromo-4-[(phenylmethyl)amino]-2(1H)-quinolinone, contaminated with the des-bromo analog. A mixture of this material, 4-(trifluoromethyl)phenylboronic acid (0.138 g, 0.727 mmol), tetrakis(triphenylphosphine)palladium (0) (0.021 g, 0.018 mmol), 2M aqueous potassium carbonate (3 mL, 6 mmol) and dioxane (3 mL) was stirred under argon at 90° C. for 24 h, then cooled, poured into 0.1 M aqueous hydrochloric acid (30 mL) and extracted with ethyl acetate. The extracts were washed with water, brine and dried (MgSO₄). The solvent was removed under reduced pressure and the residue chromatographed twice (silica gel, 3-10% methanol/dichloromethane) to give the title compound (0.063 g, 16%) as a solid. ¹H NMR (400 MHz, D₆-DMSO) δ 10.91 (s, 1H), 8.47 (d, J=1.7 Hz, 1H), 8.02 (d, J=8.2 Hz, 2H), 7.91-7.88 (m, 2H), 7.85 (d, J=8.4 Hz, 2H), 7.41-7.33 (m, 5H), 7.26 (t, J=7.1 Hz, 1H), 5.20 (d, J=1.4 Hz, 1H), 4.50 (d, J=5.8 Hz, 2H).

### b) 4-Amino-6-[4-(trifluoromethyl)phenyl]-2(1H)-quinolinone

A solution of 4-[(phenylmethyl)amino]-6-[4-(trifluoromethyl)phenyl]-2(1H)-quinolinone (0.054 g, 0.137 mmol) in acetic acid (10 mL) and concentrated aqueous hydrochloric acid (0.2 mL) was shaken with palladium-on-charcoal (10%, 0.150 g, 0.141 mmol) for 32 h under hydrogen at 50 psi, then the hydrogen replaced by nitrogen. The catalyst was filtered off through a plug of filter agent and the cake washed with methanol. The solvent was removed from the filtrate under reduced pressure and the residue chromatographed (silica gel, 7-10% methanol/dichloromethane). The product was triturated (aqueous methanol) to give the title compound (0.016 g, 38%) as a white powder. ¹H NMR (400 MHz, D₆-DMSO) δ 10.86 (s, 1H), 8.31 (s, 1H), 7.99 (d, J=8.2 Hz, 2H), 7.88 (d, J=8.4 Hz, 1H), 7.83 (d, J=8.3 Hz, 2H), 7.32 (d, J=8.6 Hz, 1H), 6.72 (s, 2H), 5.47 (s, 1H).

### EXAMPLE 74

### 6-{4-[(1-Methylethyl)sulfonyl]phenyl}-3,4-dihydro-2(1H)-quinolinone

The procedure of example 46 was followed here, using 1-bromo-4-[(1-methylethyl)sulfonyl]benzene in place of 5-bromo-2,2-difluoro-1,3-benzodioxole, to give the title compound as a solid. MS (ES) m/e 330 (M+H)⁺.

### EXAMPLE 75

### 6-[4-(Trifluoromethyl)phenyl]-1,2,3,4-tetrahydronaphthalen-1-ol

A solution of 6-[4-(trifluoromethyl)phenyl]-3,4-dihydronaphthalen-1(2H)-one (177 mg, 0.610 mmol) in THF (5 mL) at 0° C. was treated with sodium borohydride (23 mg, 0.610 mmol). After 1 h of stirring and additional 23 mg of sodium borohydride was added and the resulting solution stirred for an additional 16 h. 1N HCl (5 mL) and ethyl acetate (15 mL) were added. The organic layer was separated, washed with saturated sodium hydrogen carbonate (15 mL) and brine (15 mL), dried over MgSO₄, filtered and evaporated down to residue. Column chromatography with 0-100% step gradient of ethyl acetate in hexanes gave racemic product as a clear oil (48 mg, 27%). ¹H NMR (400 MHz, D₆-DMSO) δ 7.84-7.89 (m, 2H) 7.77-7.81 (m, 2H) 7.56 (d, J=8.59 Hz, 1H) 7.52 (d, J=1.01 Hz, 2H) 7.43 (s, 1H) 7.17-7.27 (m, 2H) 5.18 (d, J=5.81 Hz, 2H) 4.55-4.64 (m, 2H) 2.79 (d, J=3.79 Hz, 2H) 2.67-2.78 (m, 2H) 1.91-1.96 (m, 2H) 1.88 (dd, J=8.59, 4.55 Hz, 2H) 1.65-1.75 (m, 4H).

### EXAMPLE 76

### 7-(3-Fluoro-4-trifluoromethylphenyl)-1,3,4,5-tetrahydro-benzo[b]azepin-2-one

The procedure of example 46 was followed here, using 7-bromo-1,3,4,5-tetrahydro-2H-1-benzazepin-2-one (example 26a) in place of 6-bromo-1,2,3,4-tetrahydro-2-quinolinone, and 4-bromo-2-fluoro-1-(trifluoromethyl)benzene in place of 5-bromo-2,2-difluoro-1,3-benzodioxole, to give the title compound as light tan needles. MS (ES) m/e 324 (M+H)⁺.

### EXAMPLE 77

### 8-Nitro-6-[4-(trifluoromethyl)phenyl]-3,4-dihydro-2(1H)-quinolinone

### a) 6-Bromo-8-nitro-3,4-dihydro-2(1H)-quinolinone

To a stirred solution of 6-bromo-3,4-dihydro-2(1H)-quinolinone (5.83 g, 25.8 mmol) in concentrated sulfuric acid (40 mL) at 0° C. was added dropwise 90% aq nitric acid (1.3 mL 27.7 mmol.). The reaction was allowed to warm to room temperature and stirred for 2 h. The reaction was poured into ice water (300 mL), filtered, rinsed with water, and dried under vacuum. Purification by flash chromatography on silica gel (10% ethyl acetate/chloroform) gave the title compound (5.23 g, 75%) as a yellow solid: MS (ES) m/e 270.8 (M+H)⁺.

### b) 8-Nitro-6-[4-(trifluoromethyl)phenyl]-3,4-dihydro-2(1H)-quinolinone

To a stirred solution of 6-bromo-8-nitro-3,4-dihydro-2(1H)-quinolinone (1.0 g, 3.7 mmol), [4-(trifluoromethyl)phenyl]boronic acid (0.8 g, 4.2 mmol) and cesium fluoride (1.3 g, 8.4 mmol) in 1,2-dimethoxyethane (30 mL) was added tetrakis(triphenylphosphine)palladium (0) (130 mg, 0.11 mmol). The reaction was heated at reflux under nitrogen for 18 h, cooled to room temperature, diluted with water, extracted with ethyl acetate, dried (MgSO₄), filtered, and evaporated to dryness under vacuum. Purification by flash chromatography on silica gel (5 to 10% ethyl acetate/dichloromethane), trituration with hexane, filtration and drying under vacuum gave the title compound (0.91 g, 73%) as a yellow solid. ¹H NMR (400 MHz, DMSO-d₆) δ ppm 9.92 (s, 1H), 8.31 (d, J=2.0 Hz, 1H), 8.09-8.13 (m, 1H), 7.99 (d, J=8.1 Hz, 2H), 7.85 (d, J=8.3 Hz, 2H), 3.15 (t, 2H), 2.66 (t, 2H). MS (ES) m/e 337.2 (M+H)⁺.

### EXAMPLE 78

### 7-[4-(Trifluoromethyl)phenyl]-1,5-dihydro-4,1-benzothiazepin-2(3H)-one

### a) [4-nitro-4′-(trifluoromethyl)-3-biphenylyl]methanol

A mixture of 5-chloro-2 nitrobenzyl alcohol (1.2 g, 6.42 mmol), 4-(trifluoromethyl)benzeneboronic acid (1.33 g, 7.06 mmol), tetrakis(triphenylphosphine)palladium (0) (0.742 g, 0.642 mmol), and 2M aqueous potassium carbonate (12.9 mL, 25.8 mmol) in 1,4-dioxane (13 mL) was heated for 2.5 hours at 100° C. The reaction was cooled and filtered through Celite®. The filtrate was diluted with ethyl acetate, washed with water and saturated sodium chloride, dried (Na₂SO₄), concentrated in vacuo, and purified by flash chromatography on silica gel (2:1 hexanes:EtOAc) to give the title compound (1.34 g, 70%) as an off-white solid. MS (ES+) m/e 298 [M+H]⁺.

### b) 3-(Bromomethyl)-4-nitro-4′-(trifluoromethyl)biphenyl

[4-nitro-4′-(trifluoromethyl)-3-biphenylyl]methanol (0.306 g, 1.03 mmol) was dissolved in benzene (30 mL) and cooled to 0° C. Pyridine (4 drops) was added and this was followed by the dropwise addition of phosphorous tribromide (0.167 g, 0.618 mmol) in benzene (2.0 mL). The mixture was stirred overnight at room temperature. The reaction was diluted with water, extracted into diethyl ether, washed with water, dried (Na₂SO₄), and purified by flash chromatography on silica gel (3:1 hexanes:EtOAc) to give the title compound (0.125 g, 34%) as a waxy yellow solid. MS (ES+) m/e 361 [M+H]⁺.

### c) Ethyl ({[4-nitro-4′-(trifluoromethyl)-3-biphenylyl]methyl}thio)acetate

To a solution of 3-(bromomethyl)-4-nitro-4′-(trifluoromethyl)biphenyl (0.123 g, 0.342 mmol) in DMF (4.0 mL) was added ethyl-2 mercaptoacetate (0.041 g, 0.342 mmol) and solid potassium carbonate (0.047 g, 0.342 mmol) and the reaction stirred for 2.5 hours at room temperature. The reaction was quenched with water, extracted into ethyl acetate, washed with water and brine, dried (Na₂SO₄), and concentrated in vacuo to give the title compound (0.124 g, 90%) as a pale yellow crystalline solid. MS (ES+) m/e 400 [M+H]⁺.

### d) Ethyl ({[4-amino-4′-(trifluoromethyl)-3-biphenylyl]methyl}thio)acetate

To a solution of ethyl ({[4-nitro-4′-(trifluoromethyl)-3-biphenylyl]methyl}thio)acetate (0.114 g, 0.285 mmol) in acetic acid (6.0 mL) was added zinc powder (0.186 g, 2.85 mmol) and the suspension was stirred for 3.5 hours at room temperature. The reaction was filtered through Celite® and the filtrate was concentrated in vacuo to give the title compound (0.09 g, 86%) as a yellow solid. MS (ES+) m/e [M−HSCH₂CO₂Et+H]⁺.

### e) 7-[4-(Trifluoromethyl)phenyl]-1,5-dihydro-4,1-benzothiazepin-2(3H)-one

A solution of ethyl ({[4-amino-4′-(trifluoromethyl)-3-biphenylyl]methyl}thio)acetate (0.090 g, 0.244 mmol) in xylenes was refluxed for 4.0 hours, concentrated in vacuo, and purified by flash chromatography on silica gel (1:1 hexanes:EtOAc) to give the title compound (0.038 g, 48%) as a white solid. MS (ES+) m/e 324 [M+H]⁺.

### EXAMPLE 79

### (3R)-3-Amino-8-[4-(trifluoromethyl)phenyl]-2,3-dihydro-1,5-benzothiazepin-4(5H)-one trifluoroacetate

To a solution of 3-fluoro-4-nitro-4′-(trifluoromethyl)biphenyl (example 13a, 0.12 g, 0.421 mmol)) in 1-methyl-2-pyrollidinone (1 mL) was added N-(tert-butoxycarbonyl)-L-cysteine (0.093 g, 0.421 mmol) and N,N-diisopropylethylamine (0.163 g, 1.26 mmol). The reaction was stirred at room temperature for 24 h. The solvent was evaporated and the residue partitioned between ethyl acetate and water. The aqueous layer was adjusted to pH 4 with citric acid and the product extracted out with ethyl acetate. After drying (MgSO₄), the solvent was removed under reduced pressure to leave the alkylated amino-acid which was used without further purification.

The crude amino-acid was stirred in methanol (5 mL) with 10% palladium-on-charcoal (0.05 g, 0.05 mmol) under 1 atm of hydrogen for 4 h. The catalyst was filtered off and solvent evaporated to give the crude anilines, which was dissolved in DMF (3 mL). 1-[3-(Dimethylamino)propyl]-3-ethylcarbodiimide hydrochloride (0.057 g, 0.295 mmol) was added and the reaction stirred overnight. The DMF was evaporated and the residue partitioned between ethyl acetate and water. The product was purified by silica chromatography eluting with ethyl acetate/hexane. The purified product was stirred with TFA/methylene chloride 1:1 (2 mL) for 2 h at room temperature. Evaporation gave the title compound as the TFA salt. ¹H NMR (400 MHz, D6-DMSO) δ 10.74 (s, 1H), 8.34 (s, 3H), 8.01 (d, J=2.16, 1H), 7.95 (d, J=1.8 Hz, 2H), 7.82-7.88 (m, 3H), 7.30 (d, J=8.3 Hz, 1H), 4.10 (q, 1H), 3.74 (q, 1H), 3.3 (t, 1H). ES MS [M+H]⁺=339.

### EXAMPLE 80

### 6-[4-Bromophenyl)-3,4-dihyro-2(1H)-quinolinone

Following the procedure outlined in Example 65 except substituting (4-bromophenyl)boronic acid for 4-isopropyl thiophenyl boronic acid, the title compound was prepared. MS (ES) m/e 302 (M+H)⁺.

### EXAMPLE 81

### 1-(3-Methylbutanol)-6-[4-(trifluoromethyl)phenyl]-3,4-dihydroquinolin-2(1H)-one

To a solution of 1M lithium diisopropylamide (515 uL, 0.515 mmol) in tetrahydrofuran (4 mL) at 0° C. under nitrogen was added 6-[4-(trifluoromethyl)phenyl]-3,4-dihydroquinolin-2(1H)-one (example 12, 100 mg, 0.343 mmol). The reaction mixture was warmed to room temperature over 30 min and then cooled to −78° C. followed by addition of i-valeryl chloride (45.5 mg, 0.378 mmol). The resulting reaction mixture was allowed to warm to room temperature over 16 h. The reaction was quenched by the addition of saturated ammonium chloride (4 mL) and diluted with ethyl acetate (40 mL). The organic layer was separated, washed with saturated sodium bicarbonate (40 mL) and brine (40 mL), dried over MgSO₄, filtered and solvents removed under reduced pressure. The crude residue was purified on rp-HPLC and the product isolated as a brown sticky solid (13 mg). ¹H NMR (400 MHz, CHLOROFORM-d) δ ppm 7.64-7.72 (m, 4H) 7.43-7.48 (m, 2H) 7.38-7.42 (m, 1H) 2.97 (ddd, J=13.71, 6.76, 6.57 Hz, 4H) 2.74-2.80 (m, 2H) 2.31 (dt, J=13.39, 6.69 Hz, 1H) 1.05 (d, J=6.57 Hz, 6H). MS (ES+) m/e 376 [M+H]⁺.

### EXAMPLE 82

### N,N-Dimethyl-2-[2-oxo-6-[4-(trifluoromethyl)phenyl]-3,4-dihydro-1(2H)-quinolinyl]acetamide

The procedure of example 81 was followed here using 2-chloro-N,N-dimethylacetamide in place of i-valeryl chloride to give the title compound as a brown sticky solid. ¹H NMR (400 MHz, D₆-DMSO) δ 7.85-7.91 (m, 2H) 7.77-7.82 (m, 2H) 7.64 (s, 1H) 7.58 (dd, J=8.59, 2.02 Hz, 1H) 6.95 (d, J=8.59 Hz, 1H) 4.77 (s, 2H) 3.11 (s, 3H) 2.95-3.02 (m, 2H) 2.86 (s, 3H) 2.58-2.65 (m, 2H). MS (ES+) m/e 377 [M+H]⁺.

### EXAMPLE 83

### (3S)-3-Amino-8-[4-(trifluoromethyl)phenyl]-2,3-dihydro-1,5-benzoxazepin-4(5H)-one trifluoroacetate

The procedure of example 79 was followed here using N-(tert-butoxycarbonyl)-L-serine and sodium hydride in place of N-(tert-butoxycarbonyl)-L-cysteine and N,N-diisopropylethylamine respectively to give the title compound. ¹H NMR (400 MHz, D6-DMSO) δ 10.68 (s, 1H), 8.45 (s, 3H), 7.91 (d, J=8.2 Hz, 2H), 7.81 (d, J=8.2 Hz, 2H), 7.57 (m, 2H), 7.26 (d, J=7 Hz, 1H), 4.59 (m, 1H), 4.57 (m, 1H), 4.42 (t, J=10 Hz, 1H). ES MS [M+H]⁺=323.

### EXAMPLE 84

### 8-[4-(Trifluoromethyl)phenyl]-3,4,5,6-tetrahydro-1-benzazocin-2(1H)-one

### a) 8-Bromo-3,4,5,6-tetrahydro-1-benzazocin-2(1H)-one

3,4,5,6-tetrahydro-1-benzazocin-2(1H)-one (0.22 g, 1.25 mmol) in AcOH (2.0 mL) and several drops of sulfuric acid was treated dropwise with bromine (0.08 mL, 1.56 mmol) in AcOH (2.0 mL). The reaction was sealed and stirred for 12 hours at room temperature. The reaction was poured into ice and neutralized with ammonium hydroxide. The solution was then twice extracted with ethyl acetate and organics combined and washed with sat. aq. sodium bicarbonate and twice with brine. The organic layer was dried over MgSO₄, filtered and concentrated in vacuo. Material triturated with ethyl acetate to yield the title compound (0.13 g, 40%) as a white solid. ¹H NMR (400 MHz, CDCl₃) δ 7.45 (d, J=2.3 Hz, 1H), 7.33 (dd, J=2.3, 6.0 Hz, 1H), 6.95 (d, J=8.3 Hz, 1H), 2.67 (bm, 3H), 2.16 (bm, 2H), 1.85 (bm, 4H).

### b) 8-[4-(Trifluoromethyl)phenyl]-3,4,5,6-tetrahydro-1-benzazocin-2(1H)-one

The procedure of example 12 was followed here using 8-bromo-3,4,5,6-tetrahydro-1-benzazocin-2(1H)-one in place of 6-bromo-3,4-dihydro-1H-quinolin-2-one to give the title compound (8%) as a white solid. ¹H NMR (400 MHz, MeOD) δ 7.87 (d, J=8.3 Hz, 2H), 7.77 (d, J=8.3 Hz, 2H), 7.70 (d, J=2.1 Hz, 1H), 7.59 (dd, J=2.1, 6.0 Hz, 1H), 7.24 (d, J=8.1 Hz, 1H), 2.99 (bm, 1H), 2.55 (bm, 1H), 2.22 (bm, 2H), 2.07 (bm, 2H), 1.76 (bm, 1H), 1.56 (bm, 1H).

### EXAMPLE 85

### ±3-Hydroxy-6-[4-(trifluoromethyl)phenyl]-3,4-dihydro-2(1H)-quinolinone

### a) ±3-Hydroxy-3,4-dihydro-2(1H)-quinolinone

A solution of 2-nitrophenylpyruvic acid (1.05 g, 5.00 mmol) in 1M aqueous sodium hydroxide (5.00 mL, 5.00 mmol) was added over 5 min to an ice-cooled, stirred solution of sodium borohydride (0.063 g, 1.67 mmol) in water (2 mL) and the mixture stirred 1 h. 1M aqueous hydrochloric acid (10 mL) was added slowly and stirring continued 0.5 h. The mixture was extracted with ethyl acetate, and the extracts washed with brine, dried (MgSO₄) and evaporated to dryness under reduced pressure. A solution of the crude alcohol in methanol (10 mL) was stirred under 1 atm of hydrogen with 5% palladium-on-charcoal (0.170 g, 0.080 mmol) for 2 h, then the hydrogen flushed out with nitrogen. The mixture was filtered through filter agent and the cake washed with methanol. The solvent was removed from the filtrate under reduced pressure to leave the title compound (0.711 g, 87%) as a pale purple solid. ¹H NMR (400 MHz, D₆-DMSO) δ 10.13 (br s, 1H), 7.18 (d, J=7.6 Hz, 1H), 7.15 (t, J=7.7 Hz, 1H), 6.92 (td, J=7.5, 1.1 Hz, 1H), 6.85 (dd, J=7.9, 0.7 Hz, 1H), 5.42 (d, J=4.2 Hz, 1H), 4.12 (m, 1H), 3.04 (dd, J=15.5, 6.1 Hz, 1H), 2.84 (dd, J=15.4, 11.7 Hz, 1H).

### b) ±6-Bromo-3-hydroxy-3,4-dihydro-2(1H)-quinolinone

A solution of N-bromosuccinimide (0.132 g, 0.742 mmol) in dimethylformamide (1 mL) was added dropwise to a stirred solution of ±3-hydroxy-3,4-dihydro-2(1H)-quinolinone (0.110 g, 0.674 mmol) in dimethylformamide (2 mL) at room temperature and stirring continued for 1 h. 0.5M aqueous sodium bicarbonate solution (50 mL) was added and the mixture extracted with ethyl acetate. The extracts were washed with brine, dried (Na₂SO₄) and the solvent removed under reduced pressure to give the title compound (0.160 g, 98%) as a solid. ¹H NMR (400 MHz, D₆-DMSO) δ 10.24 (a, 1H), 7.41 (d, J=1.9 Hz, 1H), 7.33 (dd, J=8.4, 2.2 Hz, 1H), 6.80 (d, J=8.4 Hz, 1H), 5.53 (d, J=4.8 Hz, 1H), 4.12 (m, 1H), 3.06 (dd, J=15.7, 6.0 Hz, 1H), 2.85 (dd, J=15.7, 11.2 Hz, 1H).

### c) ±3-Hydroxy-6-[4-(trifluoromethyl)phenyl]-3,4-dihydro-2(1H)-quinolinone

A mixture of ±6-bromo-3-hydroxy-3,4-dihydro-2(1H)-quinolinone (0.158 g, 0.653 mmol), 4-(trifluoromethyl)phenylboronic acid (0.248 g, 1.31 mmol), tetrakis(triphenylphosphine)palladium (0) (0.038 g, 0.033 mmol), 2M aqueous potassium carbonate (5 mL, 10 mmol) and dioxane (5 mL) was stirred under argon at 90° C. for 18 h, then cooled, poured into water (50 mL) and extracted with ethyl acetate. The extracts were washed with water, brine and dried (Na₂SO₄), then the solvent removed under reduced pressure and the residue chromatographed (silica gel, 8% methanol/dichloromethane+1% concentrated aqueous ammonia solution). The product was re-precipitated from methanol with water and dried to give the title compound (0.113 g, 56%) as a solid. ¹H NMR (400 MHz, D₆-DMSO) δ 10.30 (s, 1H), 7.86 (d, J=8.3 Hz, 2H), 7.78 (d, J=8.4 Hz, 2H), 7.63 (d, J=1.7 Hz, 1H), 7.57 (dd, J=8.3, 2.1 Hz, 1H), 6.97 (d, J=8.3 Hz, 1H), 5.53 (d, J=4.4 Hz, 1H), 4.19 (m, 1H), 3.16 (dd, J=15.5, 5.3 Hz, 1H), 2.94 (dd, J=15.5, 11.5 Hz, 1H).

### EXAMPLE 86

### 7-[4-(Trifluoromethyl)phenyl]-1,3,4,5-tetrahydro-2H-1,5-benzodiazepin-2-one

### a) Ethyl N-[4-nitro-4′-(trifluoromethyl)-3-biphenylyl]-β-alaninate

The procedure of example 13b was followed here using β-alanine ethyl ester hydrochloride in place of 1,1-dimethylethyl glycinate hydrochloride to give the title compound (99%) as a yellow powder. ¹H NMR (400 MHz, CDCl₃) δ 8.28 (d, J=8.85 Hz, 1H), 7.72 (dd, J=8.6, 6.0 Hz, 4H), 7.02 (d, J=1.6 Hz, 1H), 6.88 (dd, J=1.8, 7.0 Hz, 1H), 4.20 (dd, 2H), 3.77 (m, 2H), 2.75 (t, 2H), 1.28 (t, 3H).

### b) Ethyl N-[4-amino-4′-(trifluoromethyl)-3-biphenylyl]-β-alaninate

Ethyl N-[4-nitro-4′-(trifluoromethyl)-3-biphenylyl]-β-alaninate (0.80 g, 2.09 mmol) was charged to a 50 mL round bottom and 10% palladium-on-charcoal (0.112 g) added under nitrogen followed by addition of methanol (10.0 mL). The reaction vessel was purged and stirred for 1 hour under an atmospheric pressure of hydrogen. The palladium was removed via filtration and solution concentrated under reduced pressure. Column chromotography (1:1 hexane/ethyl acetate) provided the title compound (0.5 g, 67%) as a white solid. ¹H NMR (400 MHz, CDCl₃) δ 7.63 (s, 4H), 6.97 (dd, J=1.9. 5.9 Hz, 1H), 6.90 (d, J=7.9 Hz, 1H), 4.16 (q, 2H), 3.76 (bs, 1H), 3.49 (t, 2H), 1.28 (t, 3H).

### c) 7-[4-(Trifluoromethyl)phenyl]-1,3,4,5-tetrahydro-2H-1,5-benzodiazepin-2-one

Ethyl N-[4-amino-4′-(trifluoromethyl)-3-biphenylyl]-β-alaninate (0.10 g, 0.28 mmol) was dissolved in acetic acid (10.0 mL) and heated to 90° C. for 2 hr. The reaction mixture was concentrated under reduced pressure. Column chromotography (1:1 hexane/ethyl acetate to 100% ethyl acetate) provided the title compound (41.9 mg, 48%) as a white solid. ¹H NMR (400 MHz, CDCl₃) δ 8.95 (bs, 1H), 7.66 (m, 4H), 7.05 (m, 2H), 6.96 (s, 1H), 4.15 (bs, 1H), 3.70 (t, 2H), 2.81 (t, 2H). ES MS [M+H]⁺=307.

### EXAMPLE 87

### 1,1-Dimethylethyl 4-oxo-8-[4-(trifluoromethyl)phenyl]-3,4-dihydro-1,5-benzothiazepine-5(2H)-carboxylate

A mixture of 8-[4-(trifluoromethyl)phenyl]-2,3-dihydro-1,5-benzothiazepin-4(5H)-one (example 55, 0.068 g, 0.21 mmol), triethylamine (0.024 g, 0.232 mmol), 4-dimethylaminopyridine (0.0026 g, 0.021 mmol), and di-tert-butyl dicarbonate (0.074 g, 0.338 mmol) in methylene chloride (3 mL) was stirred for 72 hours at room temperature. The reaction was washed sequentially with 1N HCl, water, 5% sodium bicarbonate, water, and brine, dried (Na₂SO₄), and purified by flash chromatography on silica gel (60:40 hexanes:EtOAc) to give the title compound (0.057 g, 64%) as a white solid. MS (ES+) m/e 424 [M+H]⁺.

### EXAMPLE 88

### N-[5-(2-oxo-1,2,3,4-tetrahydro-6-quinolinyl)-2-(trifluoromethyl)phenyl]acetamide

A mixture of 6-[3-amino-4-(trifluoromethyl)phenyl]-3,4-dihydro-2(1H)-quinolinone (Example 68, 0.020 g, 0.065 mmol) and acetic anhydride (2 mL) was heated under microwave irradiation at 100° C. for thirty minutes. The mixture was cooled and the precipitate was filtered and washed with ether to afford the title compound as a white solid (10 mg, 44%). ¹H NMR (400 MHz, CD₃OD) δ 7.8 (m, 2H), 7.7 (d, J=8.0 Hz, 1H), 7.5 (m, 2H), 7.0 (d, J=7.0 Hz, 1H), 3.1 (t, J=7.5 Hz, 2H), 2.6 (t, J=7.5 Hz, 2H), 2.2 (s, 3H). MS (ES+) m/e 349 [M+H]⁺.

### EXAMPLE 89

### N-{(3S)-4-Oxo-8-[4-(trifluoromethyl)phenyl]-2,3,4,5-tetrahydro-1,5-benzoxazepin-3-yl}acetamide

(3S)-3-Amino-8-[4-(trifluoromethyl)phenyl]-2,3-dihydro-1,5-benzoxazepin-4(5H)-one trifluoroacetate (example 83, 0.100 g, 0.31 mmole) was dissolved in 5 mL of methylene chloride and N,N-diisopropylethylamine (0.129 g, 1.00 mmol) was added followed by acetyl chloride (0.028 g, 0.35 mmol). The reaction was stirred 6 h at room temperature, then washed with 10% aqueous sodium bicarbonate and the crude product purified by silica gel chromatography, eluting with methylene chloride/methanol, to give the title compound (0.05 g, 45%) as a solid. ¹H NMR (400 MHz, D6-DMSO) δ 10.19 (s, 1H), 8.29 (d, J=8.2 Hz, 1H), 7.91 (d, J=8.2 Hz, 2H), 7.8 (d, J=8.2 Hz, 2H), 7.55 (d, J=8.2 Hz, 1H), 7.53 (s, 1H), 7.22 (d, J=8.2 Hz, 1H), 4.75 (m, 1H), 4.39 (m, 1H), 4.27 (t, J=10 Hz, 1H), 1.88 (s, 3H). ESMS [M+H]⁺=365.

### EXAMPLE 90

### N-{(3S)-4-Oxo-8-[4-(trifluoromethyl)phenyl]-2,3,4,5-tetrahydro-1,5-benzoxazepin-3-yl}methanesulfonamide

The procedure of example 89 was followed here using methanesulfonyl chloride in place of acetyl chloride to give the title compound (28%) as a solid. ¹H NMR (400 MHz, D6-DMSO) δ 10.26 (s, 1H), 7.91 (d, J=8.2 Hz, 2H), 7.8 (d, J=8.2 Hz, 2H), 7.56 (b s, 1H), 7.55 (d, J=8.2 Hz, 1H), 7.53 (s, 1H), 7.23 (d, J=8.2 Hz, 1H), 4.46 (m, 1H), 4.38 (m, 1H), 4.28 (t, J=10 Hz, 1H), 3.33 (s, 3H). ESMS [M+H]⁺=401.

### EXAMPLE 91

### 1-Acetyl-6-[4-(trifluoromethyl)phenyl]-3,4-dihydro-2(1H)-quinolinone

A mixture of 4-dimethylaminopyridine (15 mg, 0.123 mmol), triethylamine (58 μL, 0.412 mmol), 6-[4-(trifluoromethyl)phenyl]-3,4-dihydroquinolin-2(1H-one (example 12, 100 mg, 0.343 mmol), acetyl chloride (29 mg, 0.378 mmol) in dichloromethane (2 mL) was stirred at room temperature over 16 h. The reaction was quenched by the addition of 1N HCl (4 mL) and diluted with dichloromethane (5 mL). The organic layer was separated, washed with saturated sodium bicarbonate (10 mL), dried over MgSO₄, filtered and solvents removed under reduced pressure. Column chromatography with 0-100% step gradient of ethyl acetate in hexanes gave the desired product as an off white powder (9 mg, 7.5%). ¹H NMR (400 MHz, CDCl₃-d) δ ppm 7.71 (d, J=2.53 Hz, 4H) 7.53-7.57 (m, 1H) 7.51 (d, J=2.02 Hz, 1H) 7.47 (s, 1H) 2.97-3.03 (m, 2H) 2.78-2.85 (m, 2H) 2.74 (s, 3H). MS (ES+) m/e 334 [M+H]⁺.

### EXAMPLE 92

### 7-[4-(Trifluoromethyl)phenyl]-1,5-dihydro-4,1-benzoxazepin-2(3H)-one

### a) [4-Amino-4′-(trifluoromethyl)-3-biphenylyl]methanol

Zinc powder (1.57 g, 24.0 mmol) was added in portions to a solution of [4-nitro-4′-(trifluoromethyl)-3-biphenylyl]methanol (example 6b, 0.713 g, 2.4 mmol) in acetic acid (20.0 mL) and the suspension was stirred for 1.0 h at room temperature. The reaction was filtered through Celite® and the filtrate was concentrated in vacuo. The residue was triturated with water, filtered, and dried in a Buchner funnel to give the title compound (0.61 g, 95%) as a tan solid. MS (ES+) m/e 268 [M+H]⁺.

### b) 2-Chloro-N-[3-(hydroxymethyl)-4′-(trifluoromethyl)-4-biphenylyl]acetamide

[4-amino-4′-(trifluoromethyl)-3-biphenylyl]methanol (0.594 g, 2.22 mmol) and triethyl amine (0.449 g, 4.44 mmol) were dissolved in diethyl ether (20 mL) and a solution of chloroacetyl chloride (0.251 g, 2.22 mmol) in diethyl ether (5 mL) was added dropwise. The reaction was stirred overnight at room temperature, concentrated in vacuo, and purified by flash chromatography on silica gel (2:1 hexanes:EtOAc) to give the title compound (0.158 g, 21%) as a white solid. MS (ES+) m/e 344 [M+H]⁺.

### c) 7-[4-(Trifluoromethyl)phenyl]-1,5-dihydro-4,1-benzoxazepin-2(3H)-one

Sodium hydride (0.036 g of 60%, 0.902 mmol) was added to a solution of 2-Chloro-N-[3-(hydroxymethyl)-4′-(trifluoromethyl)-4-biphenylyl]acetamide (0.155 g, 0.451 mmol) in dry THF (6.0 mL) and the reaction was stirred overnight at room temperature. The reaction was concentrated in vacuo and the residue was triturated with water. The resulting solid was filtered, washed with water, and dried to give the title compound (0.127 g, 92%) as a pale yellow solid. MS (ES+) m/e 308 [M+H]⁺.

### EXAMPLE 93

### Phenylmethyl 2-oxo-6-[4-(trifluoromethyl)phenyl]-3,4-dihydro-1(2H)-quinolinecarboxylate

A solution of 6-[4-(trifluoromethyl)phenyl]-3,4-dihydroquinolin-2(1H)-one (example 12, 100 mg, 0.343 mmol) in tetrahydrofuran (5 mL) was cooled to −78° C. n-Butyl lithium, 1.6M in hexanes, (257 μL, 0.412 mmol) was added dropwise and the resulting solution stirred for 30 min. Reaction mixture became homogeneous and a solution of benzylchloroformate (73 μL, 0.515 mmol) in tetrahydrofuran (2 mL) was added dropwise. The solution was stirred for 1 h and the temperature rose to −50° C. The reaction was quenched by the addition of saturated ammonium chloride (5 mL) and diluted with ethyl acetate (40 mL) and water (30 mL). The organic layer was separated, washed with saturated sodium bicarbonate (40 mL) and brine (40 mL), dried over MgSO₄, filtered and solvents removed under reduced pressure. Column chromatography with 0-100% step gradient of ethyl acetate in hexanes gave a clear oil and upon trituration with diethyl ether the desired product was obtained as a white powder (90 mg, 62%). ¹H NMR (400 MHz, CHLOROFORM-d) δ ppm 7.66 (q, J=8.42 Hz, 4H) 7.44-7.50 (m, 2H) 7.34-7.43 (m, 5H) 7.02 (d, J=8.34 Hz, 1H) 5.43 (s, 2H) 2.98-3.07 (m, 2H) 2.74 (dd, J=8.08, 6.06 Hz, 2H).

### EXAMPLE 94

### 5-(3-Methylbutanoyl)-8-[4-(trifluoromethyl)phenyl]-2,3-dihydro-1,5-benzothiazepin-4(5H)-one

A mixture of 8-[4-(trifluoromethyl)phenyl]-2,3-dihydro-1,5-benzothiazepin-4(5H)-one (example 55, 0.068 g, 0.21 mmol), triethylamine (0.024 g, 0.232 mmol), 4-dimethylaminopyridine (0.0026 g, 0.021 mmol), and isovaleryl chloride (0.051 g, 0.425 mmol) in methylene chloride (4 mL) was stirred for 5.0 hours at room temperature. The reaction was washed sequentially with 1N HCl, water, 5% sodium bicarbonate, water, and brine, dried (Na₂SO₄), and purified by flash chromatography on silica gel (2:1 hexanes:EtOAc) to give the title compound (0.057 g, 64%) as a white solid. MS (ES+) m/e 408 [M+H]⁺.

### EXAMPLE 95

### 6-[3,5-difluoro-4-(trifluoromethyl)phenyl]-3,4-dihydro-2(1H)-quinolinone

To a solution of (2-oxo-1,2,3,4-tetrahydro-6-quinolinyl)boronic acid (example 59a, 109 mg, 0.57 mmol) in dioxane (5 mL) and aqueous potassium carbonate (2 M, 5 mL) were added tetrakis(triphenylphosphine)palladium(0) (33 mg, 0.028 mmol) and 5-bromo-1,3-difluoro-2-(trifluoromethyl)benzene (150 mg, 0.57 mmol). The mixture was heated at 90° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (12 mg, 7%) as a white solid. ¹H NMR (400 MHz, CDCl₃) δ 8.39 (br s, 1H), 7.43 (m, 2H), 7.22 (d, J=0.8 Hz, 1H), 7.19 (s, 1H), 6.91 (d, J=8.9 Hz, 1H), 3.09 (t, J=7.3 Hz, 2H), 2.73 (t, J=7.6 Hz, 2H). MS (ES+) m/e 328[M+H]⁺.

### EXAMPLE 96

### ±3-Methyl-7-[4-(trifluoromethyl)phenyl]-1,3,4,5-tetrahydro-2H-1,5-benzodiazepin-2-one

A solution of 3-fluoro-4-nitro-4′-(trifluoromethyl)biphenyl (example 13a, 0.410 g, 1.45 mmol) in dioxane (5.0 mL) was treated with N,N-diisopropylethylamine (0.5 mL, 2.87 mmol) and DL-3-aminoisobutyric acid (0.20 g, 1.93 mmol). The mixture was stirred at 95° C. for 18 h, cooled and evaporated under reduced pressure. The residue was diluted with ethyl acetate and washed with 1M aqueous HCl, H₂O, and then brine, dried over MgSO₄, and filtered. The filtrate was concentrated and then redissolved in CH₂Cl₂. N-hydroxysuccinimide (0.18 g, 1.58 mmol) and 1,3-diisopropylcarbodiimide (0.23 mL, 1.47 mmol) were added and the mixture allowed to stir overnight. After concentrating, column chromatography (4:1 hexane:ethyl acetate) provided a yellow solid. The solid was charged to a 50 mL round bottom and 10% Pd/C added followed by methanol (16 mL). The reaction vessel was purged and stirred under atmospheric pressure of hydrogen. After 1 hour LC/MS confirmed progress toward cyclized material. The reaction was filtered, rediluted in CH₂Cl₂ (10.0 mL) and treated with diisopropylethylamine (0.5 mL, 2.87 mmol). After 3 days, the mixture was concentrated under reduced pressure. Column chromotography (100% ethyl acetate to 10% methanol/dichloromethane) provided the title compound (0.084 mg, 61%) as a white solid. ¹H NMR (400 MHz, CDCl₃) δ 7.75 (dd, J=8.5, 15.6 Hz, 4H), 7.20 (d, J=2.0 Hz, 1H), 7.11 (dd, J=2.2, 6.0 Hz, 1H), 7.03 (d, J=8.0 Hz, 1H), 3.60 (dd, 1H) 3.35 (dd, 1H), 2.83 (m, 1H), 1.13 (d, 3H).

### EXAMPLE 97

### (3R)-3-Amino-8-[4-(trifluoromethyl)phenyl]-2,3-dihydro-1,5-benzoxazepin-4(5H)-one trifluoroacetate

The procedure of example 83 was followed here using N-(tert-butoxycarbonyl)-D-serine in place of N-(tert-butoxycarbonyl)-L-serine to give the title compound. ¹H NMR (400 MHz, D6-DMSO) δ 10.68 (s, 1H), 8.45 (s, 3H), 7.91 (d, J=8.2 Hz, 2H), 7.81 (d, J=8.2 Hz, 2H), 7.57 (m, 2H), 7.26 (d, J=7 Hz, 1H), 4.59 (m, 1H), 4.57 (m, 1H), 4.42 (t, J=10 Hz, 1H). ESMS [M+H]⁺=323.

### EXAMPLE 98

### 7-[4-(Trifluoromethyl)phenyl]-1,3-dihydro-2H-1,4-benzodiazepin-2-one

### a) 4-Amino-4′-(trifluoromethyl)-3-biphenylcarbaldehyde

A mixture of 5-chloro-2-nitrobenzaldehyde (3.71 g, 20.0 mmol), 4-(trifluoromethyl)phenylboronic acid (6.41 g, 33.7 mmol), tetrakis(triphenylphosphine)palladium (0) (1.16 g, 1.00 mmol), 2M aqueous potassium carbonate (60 mL, 120 mmol) and dioxane (60 mL) was stirred while heating under reflux under nitrogen for 8 h, then cooled, poured into water (600 mL) and extracted with ethyl acetate. The extracts were washed with 0.1M aqueous hydrochloric acid and brine, then dried (MgSO₄) and the solvent removed under reduced pressure. The residue was chromatographed twice (silica gel, 10-30% ethyl acetate/hexane) to give a small amount of the title compound (0.128 g, 2%) as a solid: ¹H NMR (400 MHz, CDCl₃) δ 9.97 (s, 1H), 7.74 (d, J=2.2 Hz, 1H), 7.67 (d, J=8.6 Hz, 2H), 7.64 (d, J=8.6 Hz, 2H), 7.60 (dd, J=8.6, 2.3 Hz, 1H), 6.77 (d, J=8.6 Hz, 1H), 6.26 (br s, 2H). The major product (3.17 g, 54%) was 4-nitro-4′-(trifluoromethyl)-3-biphenylcarbaldehyde: ¹H NMR (400 MHz, CDCl₃) δ 10.53 (s, 1H), 8.26 (d, J=8.5 Hz, 1H), 8.16 (d, J=2.1 Hz, 1H), 7.96 (dd, J=8.5, 2.2 Hz, 1H), 7.79 (d, J=8.9 Hz, 2H), 7.76 (d, J=8.9 Hz, 2H).

### b) 2-Azido-N-[3-formyl-4′-(trifluoromethyl)-4-biphenylyl]acetamide

Bromoacetyl bromide (0.050 mL, 0.580 mmol) was injected dropwise into a stirred solution of 4-amino-4′-(trifluoromethyl)-3-biphenylcarbaldehyde (0.128 g, 0.483 mmol) in dichloromethane (2 mL) at −30° C. under nitrogen and the mixture stirred while warming to 0° C. over 2 h. The mixture was re-cooled to −5° C. and 1M aqueous sodium hydroxide (0.58 mL, 0.58 mmol) added dropwise. Stirring was continued at room temperature for 0.25 h, then the mixture partitioned between aqueous sodium bicarbonate and ethyl acetate. The extracts were dried (MgSO₄) and the solvent removed under reduced pressure to give the impure bromo amide. A solution of this amide in acetone (3 mL) was stirred with sodium azide (0.289 g, 4.44 mmol) at room temperature for 18 h, then diluted with water (60 mL) and extracted with ethyl acetate. The extracts were washed with brine, dried (MgSO₄) and the solvent removed under reduced pressure. The residue was chromatographed (silica gel, 10-30% ethyl acetate/hexane) to give the title compound (0.118 g, 70%) as a gum. ¹H NMR (400 MHz, D₆-DMSO) δ 11.29 (s, 1H), 10.13 (s, 1H), 8.46 (d, J=8.7 Hz, 1H), 8.37 (d, J=2.3 Hz, 1H), 8.14 (dd, J=8.7, 2.3 Hz, 1H), 8.00 (d, J=8.2 Hz, 2H), 7.87 (d, J=8.3 Hz, 2H), 4.34 (s, 2H).

### c) 7-[4-(Trifluoromethyl)phenyl]-1,3-dihydro-2H-1,4-benzodiazepin-2-one

Triphenylphosphine (0.096 g, 0.366 mmol) was added to a stirred solution of 2-azido-N-[3-formyl-4′-(trifluoromethyl)-4-biphenylyl]acetamide (0.116 g, 0.333 mmol) in tetrahydrofuran/water (10:1, 4.4 mL) at room temperature and the mixture stirred 16 h, then diluted with water (20 mL). The precipitated solid was filtered off, washed with water and dried. The crude product was re-precipitated from methanol with water, filtered, washed with water, then ether and dried to give the title compound (0.059 g, 58%) as a solid. ¹H NMR (400 MHz, D₆-DMSO) δ 10.79 (s, 1H), 8.69 (s, 1H), 8.03 (d, J=2.2 Hz, 1H), 7.97-7.92 (m, 3H), 7.85 (d, J=8.3 Hz, 2H), 7.28 (d, J=8.5 Hz, 1H), 4.15 (d, J=1.0 Hz, 2H).

### EXAMPLE 99

### 8-[4-(Trifluoromethyl)phenyl]-2,3-dihydro-1,5-benzoxazepin-4(5H)-one

### a) 3-{[4-Nitro-4′-(trifluoromethyl)-3-biphenylyl]oxy}-1-propanol

A mixture of 3-Fluoro-4-nitro-4′-(trifluoromethyl)biphenyl (example 13a, 0.541 g, 1.89 mmol), 1,3-propanediol (0.144 g, 1.89 mmol), and sodium hydride (0.076 g of 60%, 1.89 mmol) in DMF (10.0 mL) was stirred overnight at room temperature. The reaction was acidified to pH 4 with 1.0M hydrochloric acid and the resulting precipitate was filtered and purified by flash chromatography on silica gel (7:3 hexanes:EtOAc) to give the title compound (0.353 g, 55%) as a pale yellow solid. MS (ES+) m/e 342 [M+H]⁺.

### b) 3-{[4-Nitro-4′-(trifluoromethyl)-3-biphenylyl]oxy}propanoic acid

3-{[4-Nitro-4′-(trifluoromethyl)-3-biphenylyl]oxy}-1-propanol (0.218 g, 0.639 mmol) was dissolved in acetone (5.0 mL) and cooled to 0° C. Jones reagent was added dropwise until an orange color persisted and the reaction was stirred for 2.0 hours at room temperature. The reaction was quenched with dropwise addition of isopropanol and the resulting solid was filtered. The filtrate was concentrated in vacuo to give the title compound (0.180 g, 79%) as a tan solid. MS (ES+) m/e 356 [M+H]⁺.

### c) 3-{[4-Amino-4′-(trifluoromethyl)-3-biphenylyl]oxy}propanoic acid

10% palladium/carbon was added to a solution of 3-{[4-nitro-4′-(trifluoromethyl)-3-biphenylyl]oxy}propanoic acid (0.210 g, 0.591 mmol) in ethyl acetate (5.0 mL) and the mixture was placed under a balloon of hydrogen overnight. The reaction was filtered through Celite® and concentrated to give the title compound (0.192 g, 90%) as a white solid. MS (ES+) m/e 326 [M+H]⁺.

### d) 8-[4-(trifluoromethyl)phenyl]-2,3-dihydro-1,5-benzoxazepin-4(5H)-one

3-{[4-Amino-4′-(trifluoromethyl)-3-biphenylyl]oxy}propanoic acid (0.170 g, 0.523 mmol) was dissolved in DMF (6.0 mL). 1-[3-(Dimethylamino)propyl]-3-ethylcarbodiimide hydrochloride (0.105 g, 0.549 mmol) was added and the reaction was stirred at room temperature for three days. The reaction was diluted with water and the resulting precipitate was filtered and purified by flash chromatography on silica gel (1:1 hexanes:EtOAc) to give the title compound as a white solid. MS (ES+) m/e 308 [M+H]⁺.

### EXAMPLE 100

### 8-[4-(Trifluoromethyl)phenyl]-2,3-dihydro-1,5-benzothiazepin-4(5H)-one 1,1-dioxide

A mixture of 8-[4-(trifluoromethyl)phenyl]-2,3-dihydro-1,5-benzothiazepin-4(5H)-one (example 55, 0.071 g, 0.219 mmol) and 3-chloroperoxybenzoic acid (0.113 g, 0.66 mmol) in 1,4-dioxane (6 mL) was stirred for 72 hours at room temperature. The reaction was quenched with 20% sodium thiosulfate solution followed by addition of 5% sodium bicarbonate solution. The resulting precipitate was filtered, washed with water, and dried to give the title compound (77.6 mg, 84%) as a white solid. MS (ES+) m/e 356 [M+H]⁺.

### EXAMPLE 101

### 8-Amino-6-[3-fluoro-4-(trifluoromethyl)phenyl]-3,4-dihydro-2(1H)-quinolinone

### a) 6-[3-Fluoro-4-(trifluoromethyl)phenyl]-8-nitro-3,4-dihydro-2(1H)-quinolinone

To a stirred solution of 4-bromo-2-fluoro-1-(trifluoromethyl)benzene (1.0 g, 4.1 mmol), bis(pinacolato)diboron (1.1 g, 4.3 mmol) and potassium acetate (1.2 g, 122 mmol) in dimethylformamide (50 mL) was added [1,1′-bis(diphenylphosphino)ferrocene]dichloropalladium(II)dichloromethane adduct (81 mg, 0.11 mmol). After purging with nitrogen, the reaction was heated to 80° C. and stirred for 2 h. 6-bromo-8-nitro-3,4-dihydro-2(1H)-quinolinone (example 77a, 1.0 g, 3.7 mmol), cesium fluoride (1.3 g, 8.4 mmol), and tetrakis(triphenylphosphine)palladium (0) (130 mg, 0.11 mmol) were added and the reaction stirred at 100° C. for 18 h. After cooling to room temperature, the reaction was evaporated to dryness under vacuum. The residue was taken up in ethyl acetate, filtered to remove insolubles, washed with 1M aq hydrochloric acid, brine, dried (MgSO₄), filtered and concentrated under vacuum. Purification by flash chromatography on silica gel (3% ethyl acetate/dichloromethane) gave the title compound (0.77 g, 59%) as a yellow solid. MS (ES) m/e 355.0 (M+H)⁺.

### b) 8-Amino-6-[3-fluoro-4-(trifluoromethyl)phenyl]-3,4-dihydro-2(1H)-quinolinone

To a stirred solution of 6-[3-fluoro-4-(trifluoromethyl)phenyl]-8-nitro-3,4-dihydro-2(1H)-quinolinone (0.5 g, 1.4 mmol) in acetic acid (30 mL) was added zinc dust (0.65 g, 9.9 mmol) portionwise. The reaction was stirred at room temperature for 18 h, filtered through a pad of Celite®, rinsed with acetic acid, and evaporated to dryness under vacuum. The residue was basified with 1N aq sodium carbonate, extracted with ethyl acetate, washed with brine, dried (Na₂SO₄), filtered and concentrated under vacuum. Purification by flash chromatography on silica gel (60 to 80% ethyl acetate/dichloromethane), trituration with 20% ethyl acetate/hexane, filtration and drying under vacuum gave the title compound (435 mg, 95%) as a white solid. ¹H NMR (400 MHz, DMSO-d₆) δ ppm 9.49 (s, 1H), 7.80 (t, 1H), 7.65 (d, J=12.9 Hz, 1H), 7.58 (d, J=8.3 Hz, 1H), 6.95 (d, J=2.0 Hz, 1H), 6.90 (d, J=1.8 Hz, 1H), 5.24 (s, 1H), 2.88 (t, 2H), 2.46 (t, 2H). MS (ES) m/e 325 (M+H)⁺.

### EXAMPLE 102

### 6-[4-(1,1-Dimethylpropyl)-1-cyclohexen-1-yl]-3,4-dihydro-2(1H)-quinolinone

To a microwave tube was added 6-bromo-3,4-dihydro-2(1H)-quinolinone (100 mg, 0.444 mmol), 4-(1,1-dimethylpropyl)-1-cyclohexen-1-yl trifluoromethanesulfonate (126 mg, 0.422 mmol), bis(pinacolato)diboron (124 mg, 0.488 mmol), [1,1′-bis(diphenylphosphino)ferrocene]dichloropalladium(II)dichloromethane adduct (22 mg, 0.026 mmol), potassium acetate (131 mg, 1.30 mmol), 2M aqueous potassium carbonate (650 μL, 1.30 mmol), and DMF (3 mL). The tube was heated in the microwave at 100° C. for 400 s. The reaction mixture was diluted with ethyl acetate (20 mL) and filtered. The filtrate was washed with water (20 mL) and brine (20 mL). The organic layer was dried over MgSO₄, filtered and solvent removed under reduced pressure. The crude residue was purified by rp-HPLC to give the title compound as a light brown powder (6.5 mg, 5%). ¹H NMR (400 MHz, CDCl₃) δ ppm 7.90 (s, 1H) 7.19 (d, J=3.79 Hz, 2H) 6.63-6.71 (m, 1H) 6.04-6.10 (m, 1H) 2.96 (t, J=7.45 Hz, 2H) 2.58-2.67 (m, 2H) 2.44-2.47 (m, 1H) 2.39 (ddd, J=9.41, 4.74, 2.53 Hz, 1H) 2.13-2.23 (m, 1H) 1.90-2.02 (m, 2H) 1.38-1.49 (m, 1H) 1.25-1.36 (m, 4H) 0.79-0.89 (m, 9H).

### EXAMPLE 103

### [5-(2-oxo-1,2,3,4-tetrahydro-6-quinolinyl)-2-(trifluoromethyl)phenyl]acetonitrile

To a solution of (2-oxo-1,2,3,4-tetrahydro-6-quinolinyl)boronic acid (example 59a, 128 mg, 0.67 mmol) in dioxane (5 mL) were added palladium acetate(II) (2.0 mg, 0.0045 mmol), [2′,6′-bis(methyloxy)-2-biphenylyl](dicyclohexyl)phosphane (9.2 mg, 0.011 mmol), [5-chloro-2-(trifluoromethyl)phenyl]acetonitrile (100 mg, 0.45 mmol) and potassium phosphate (190.8 mg, 0.90 mmol). The mixture was heated at 80° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (26 mg, 18%) as a yellow solid. ¹H NMR (400 MHz, CDCl₃) δ 9.01 (br s, 1H), 7.86 (s, 1H), 7.76 (d, J=8.1 Hz, 1H), 7.64 (d, J=8.1 Hz, 1H), 7.47 (m, 2H), 6.97 (d, J=8.8 Hz, 1H), 4.04 (s, 2H), 3.10 (t, J=7.1 Hz, 2H), 2.74 (t, J=7.7 Hz, 2H). MS (ES+) m/e 331 [M+H]⁺.

### EXAMPLE 104

### ±2-Oxo-6-[4-(trifluoromethyl)phenyl]-1,2,3,4-tetrahydro-3-quinolinecarboxylic acid

### a) Diethyl {[4-nitro-4′-(trifluoromethyl)-3-biphenyl]methylidene}propanedioate

A mixture of 4-nitro-4′-(trifluoromethyl)-3-biphenylcarbaldehyde (example 98 (a) major product, 0.295 g, 1.00 mmol), diethyl malonate (0.167 mL, 1.10 mL), p-toluenesulfonic acid monohydrate (0.019 g, 0.100 mmol) and toluene (5 mL) was stirred at 90° C. for 60 h, then cooled and loaded on to a silica gel column. The product was eluted with 10-30% ethyl acetate/hexane to give the title compound (0.224 g, 51%) as an amorphous solid. ¹H NMR (400 MHz, CDCl₃) δ 8.33 (d, J=8.6 Hz, 1H), 8.25 (s, 1H), 7.78-7.75 (m, 3H), 7.71-7.69 (m, 3H), 4.37 (q, J=7.1 Hz, 2H), 4.09 (q, J=7.1 Hz, 2H), 1.37 (t, J=7.1 Hz, 3H), 0.99 (t, J=7.1 Hz, 3H).

### b) ±Ethyl 2-oxo-6-[4-(trifluoromethyl)phenyl]-1,2,3,4-tetrahydro-3-quinolinecarboxylate

A drop of concentrated aqueous hydrochloric acid was added every 0.25 h to a mixture of diethyl {[4-nitro-4′-(trifluoromethyl)-3-biphenylyl]methylidene}propanedioate (0.437 g, 1.00 mmol), sodium cyanoborohydride (0.315 g, 5.01 mmol) and ethanol/ethyl acetate (4:1, 5 mL) stirred at 50° C. After 1 h, the mixture was cooled, poured into 0.1M aqueous hydrochloric acid (100 mL) and extracted with ethyl acetate. The extracts were washed with brine, dried (MgSO₄) and the solvent removed under reduced pressure to give crude diethyl {[4-nitro-4′-(trifluoromethyl)-3-biphenylyl]methyl}propanedioate. A solution of this diester in methanol (10 mL) was stirred at room temperature with 10% palladium-on-charcoal (0.200 g, 0.188 mmol) under 1 atm hydrogen for 18 h, then the hydrogen replaced with nitrogen and the mixture filtered through filter agent. The cake was washed with methanol and the solvent was removed from the filtrate under reduced pressure. A solution of the residue in dichloromethane (5 mL) was added to a stirred suspension of silica gel (10 mL) impregnated with 2M aqueous sulfuric acid (˜0.1 mL) in dichloromethane (20 mL). The mixture was stirred at room temperature for 3 h, then the solvent removed under reduced pressure and the residue loaded onto a silica gel column. The product was eluted with 30-60% ethyl acetate/hexane to give the title compound (0.227 g, 62%) as a solid. ¹H NMR (400 MHz, D₆-DMSO) δ 10.58 (s, 1H), 7.86 (d, J=8.2 Hz, 2H), 7.79 (d, J=8.5 Hz, 2H), 7.64 (s, 1H), 7.58 (dd, J=8.2, 2.0 Hz, 1H), 7.00 (d, J=8.2 Hz, 1H), 4.14 (m, 2H), 3.70 (t, J=8.0 Hz, 1H), 3.23 (d, J=8.1 Hz, 2H), 1.17 (t, J=7.1 Hz, 3H).

### c) ±2-Oxo-6-[4-(trifluoromethyl)phenyl]-1,2,3,4-tetrahydro-3-quinolinecarboxylic acid

1M aqueous sodium hydroxide (1.00 mL, 1.00 mmol) was added dropwise to a stirred solution of ethyl 2-oxo-6-[4-(trifluoromethyl)phenyl]-1,2,3,4-tetrahydro-3-quinolinecarboxylate (0.034 g, 0.094 mmol) in methanol (10 mL) at room temperature. After stirring 18 h, most of the volatiles were removed under reduced pressure and the mixture diluted with water (30 mL). 1M aqueous hydrochloric acid (3 mL) was added slowly. After allowing to stand 0.5 h, the solid was filtered, washed with water and dried to give the title compound (0.027 g, 87%) as a white solid. ¹H NMR (400 MHz, D₆-DMSO) δ 12.83 (br s, 1H), 10.50 (s, 1H), 7.86 (d, J=8.2 Hz, 2H), 7.79 (d, J=8.4 Hz, 2H), 7.64 (s, 1H), 7.58 (dd, J=8.2, 2.0 Hz, 1H), 6.99 (d, J=8.2 Hz, 1H), 3.55 (t, J=7.3 Hz, 1H), 3.22 (d, J=7.3 Hz, 2H).

### EXAMPLE 105

### (3S)-3-Methyl-7-[4-(trifluoromethyl)phenyl]-1,3,4,5-tetrahydro-2H-1,4-benzodiazepin-2-one

### a) Methyl N-{[4-amino-4′-(trifluoromethyl)-3-biphenylyl]methyl}-L-alaninate

4-Nitro-4′-(trifluoromethyl)-3-biphenylcarbaldehyde (example 98a major product, 0.380 g, 1.29 mmol) and L-alanine methyl ester hydrochloride (0.200 g, 1.433 mmol), in methanol (6 mL) and acetic acid (approx 0.1 mL) were stirred for 0.5 h at room temperature. Sodium cyanoborohydride (0.162 g, 2.578 mmol) was added at 0° C. The reaction was allowed to warm slowly to room temperature and stirred overnight under nitrogen. The reaction mixture was poured into saturated sodium carbonate and twice extracted with ethyl acetate. The organics were combined and washed with brine, dried over MgSO₄, filtered, and concentrated to provided a yellow solid which was taken forward. The crude mixture was charged to a 50 mL round bottom and 10% palladium on carbon (approx. 0.15 g) was added followed by ethyl acetate (10.0 mL). The reaction vessel was purged and allowed to stir under an atmospheric pressure of hydrogen for 3 hours. The reaction mixture was filtered and concentrated. Column chromotography (4:1 hexane:ethyl acetate to 100% ethyl acetate) provided the title compound (0.080 g, 18%) as a white solid. ¹H NMR (400 MHz, D₆-DMSO) δ 8.63 (s, 1H), 8.47 (s, 1H), 7.82 (d, 2H), 7.75 (d, 2H), 7.59 (dd, 1H), 7.46 (d, 1H), 7.19 (d, 1H), 3.66 (s, 3H), 3.60 (m, 1H), 3.31 (q, 2H), 1.23 (d, 3H).

### b) (3S)-3-Methyl-7-[4-(trifluoromethyl)phenyl]-1,3,4,5-tetrahydro-2H-1,4-benzodiazepin-2-one

Methyl N-{[4-amino-4′-(trifluoromethyl)-3-biphenylyl]methyl}-L-alaninate (0.080 g, 0.228 mmol) in toluene was treated with trimethylaluminum (0.48 mL, 2.0 M in hexane) dropwise at 0° C. The reaction was allowed to slowly warm to room temperature and then stirred for several hours. The reaction was then recooled to 0° C. and quenched with methanol and the ice bath removed. After ten minutes the crude reaction mixture was diluted with ethyl acetate and sodium bicarbonate. After an additional 10 minute the solid was filtered off and the organic separated and dried over MgSO₄, filtered, and concentrated under reduced pressure. Column chromotography (100% ethyl acetate to 10% methanol/dichloromethane) yielded the title compound (0.02 g, 27% yield) as a white solid. ¹H NMR (400 MHz, D₆-DMSO) δ 9.94 (s, 1H), 7.88 (d, J=8.3 Hz, 2H), 7.81 (d, J=8.1 Hz, 2H), 7.65 (m, 2H), 7.15 (d, J=8.8 Hz, 1H), 3.87 (m, 2H), 3.30 (d, 3H), 2.94 (m, 1H).

### EXAMPLE 106

### 2-Oxo-6-[4-(trifluoromethyl)phenyl]-1,2,3,4-tetrahydro-8-quinolinecarboxylic acid

### a) 8-Bromo-6-[4-(trifluoromethyl)phenyl]-3,4-dihydro-2(1H)-quinolinone

A solution of N-bromosuccinimide (0.057 g, 0.320 mmol) and 6-(4-trifluoromethylphenyl)-3,4-dihydro-1H-quinolin-2-one (example 12, 0.085 g, 0.292 mmol) in dimethylformamide (1.5 mL) was stirred at 60° C. for 2 h, then cooled and diluted slowly with water (15 mL). The precipitate was filtered, washed with water and dried, then chromatographed (silica gel, 30-50% ethyl acetate/hexane) to give the title compound (0.072 g, 67%) as a solid. ¹H NMR (400 MHz, D₆-DMSO) δ 9.30 (s, 1H), 7.91 (d, J=8.2 Hz, 2H), 7.87 (d, J=1.9 Hz, 1H), 7.80 (d, J=8.3 Hz, 2H), 7.68 (d, J=1.6 Hz, 1H), 3.04 (m, 2H), 2.56 (m, 2H).

### b) 2-Oxo-6-[4-(trifluoromethyl)phenyl]-1,2,3,4-tetrahydro-8-quinolinecarboxylic acid

Sodium hydride (0.050 g of a 60% oil suspension, 1.25 mmol) was added to an ice-cooled, stirred solution of 8-bromo-6-[4-(trifluoromethyl)phenyl]-3,4-dihydro-2(1H)-quinolinone (0.370 g, 1.00 mmol) in tetrahydrofuran (12 mL) under argon and the mixture stirred 0.5 h, then cooled to −78° C. A solution of n-butyllithium in hexanes (2.5M, 0.500 mL, 1.25 mmol) was injected dropwise and the mixture stirred 2 h. Powdered dry ice (˜1 g) was added and the mixture warmed to room temperature, then poured into water (120 mL) and extracted with ethyl acetate. The extracts were washed with brine, dried (MgSO₄) and the solvent removed under reduced pressure. The residue was chromatographed (silica gel, 10% methanol/dichloromethane, then 10% methanol/dichloromethane+0.5% acetic acid) to give the title compound (0.024 g, 21%) as a solid. ¹H NMR (400 MHz, D₆-DMSO) δ 13.94 (br s, 1H), 11.04 (br s, 1H), 8.15 (d, J=1.8 Hz, 1H), 7.90 (d, J=8.2 Hz, 2H), 7.86 (s, 1H), 7.81 (d, J=8.3 Hz, 2H), 3.07 (m, 2H), 2.59 (m, 2H).

### EXAMPLE 107

### 2-{4-oxo-8-[4-(trifluoromethyl)phenyl]-2,3,4,5-tetrahydro-1H-1,5-benzodiazepin-1-yl}acetamide

7-[4-(Trifluoromethyl)phenyl]-1,3,4,5-tetrahydro-2H-1,5-benzodiazepin-2-one (example 86, 0.050 g, 0.163 mmol) in 1,4-dioxane (2.0 mL) was treated with 2-bromoacetamide (0.23 g, 1.667 mmol) and sodium carbonate (0.17 g, 1.604 mmol) and heated to 100° C. overnight in a sealed tube. The reaction mixture was cooled, diluted with water, and twice extracted with ethyl acetate. Organics were combined and washed with brine, dried over MgSO₄, filtered, and concentrated under reduced pressure. Column chromotography (100% ethyl acetate to 10% methanol/dichloromethane) provided the title compound (0.0145 g, 24% yield) as a yellow solid. ¹H NMR (400 MHz, D₆-DMSO) δ 9.65 (bs, 1H), 7.81 (d, J=2.6 Hz, 4H), 7.32 (bs, 1H), 7.27 (m, 2H), 7.18 (bs, 1H), 7.05 (d, J=8.04 Hz, 1H), 3.85 (s, 2H), 3.57 (t, 2H), 2.45 (t, 2H).

### EXAMPLE 108 AND 109

The racemic compound ±3-hydroxy-6-[4-(trifluoromethyl)phenyl]-3,4-dihydro-2(1H)-quinolinone (example 85) was resolved by supercritical fluid chromatography on a Chiralpak AS-H column (20% methanol/80% CO₂) to give (+) 3-Hydroxy-6-[4-(trifluoromethyl)phenyl]-3,4-dihydro-2(1H)-quinolinone, [a]_{D}+135° (c 0.2, MeOH) and (−) 3-Hydroxy-6-[4-(trifluoromethyl)phenyl]-3,4-dihydro-2(1H)-quinolinone, [a]_{D}−130° (c 0.2, MeOH), with NMR data the same as for the racemic compound.

### EXAMPLE 110

### 6-[4-(1,1-Dimethylethyl)-1-cyclohexen-1-yl]-3,4-dihydro-2(1H)-quinolinone

The title compound was prepared following the procedure described in example 102 except substituting 4-(1,1-dimethylethyl)-1-cyclohexen-1-yl trifluoromethanesulfonate for 4-(1,1-dimethylpropyl)-1-cyclohexen-1-yl trifluoromethanesulfonate. MS (ES+) m/e 284 [M+H]⁺.

### EXAMPLE 111

### 6-(4-Ethyl-1-cyclohexen-1-yl)-3,4-dihydro-2(1H)-quinolinone

The title compound was prepared following the procedure described in example 102 except substituting 4-ethyl-1-cyclohexen-1-yl trifluoromethanesulfonate for 4-(1,1-dimethylpropyl)-1-cyclohexen-1-yl trifluoromethanesulfonate. MS (ES+) m/e 256 [M+H]⁺.

### EXAMPLE 112

### 6-(4-Bromo-3-nitrophenyl)-3,4-dihydro-2(1H)-quinolinone

To a solution of (2-oxo-1,2,3,4-tetrahydro-6-quinolinyl)boronic acid (example 59a, 67 mg, 0.36 mmol) in dioxane (5 mL) and aqueous potassium carbonate (2 M, 5 mL) were added tetrakis(triphenylphosphine)palladium(0) (20 mg, 0.018 mmol) and 1,4-dibromo-2-nitrobenzene (100 mg, 0.36 mmol). The mixture was heated at 90° C. overnight, cooled, poured into water and extracted with ethyl acetate (3×30 mL). The combined extracts were dried over MgSO₄, concentrated in vacuo and purified by flash chromatography (0-100% ethyl acetate in hexanes) to give the title compound (22 mg, 18%) as a yellow solid. ¹H NMR (400 MHz, CD₃OD) δ 8.08 (d, J=1.8 Hz, 1H), 7.86 (dd, J=2.0, 8.3 Hz, 1H), 7.45 (d, J=8.0 Hz, 1H), 7.19 (d, J=1.5 Hz, 1H), 7.15 (dd, J=2.0, 8.1 Hz, 1H), 6.94 (d, J=8.3 Hz, 1H), 3.01 (t, J=7.4 Hz, 2H), 2.63 (t, J=7.3 Hz, 2H). MS (ES+) m/e 347 [M+H]⁺.

### EXAMPLE 113

### 6-(4-Bromo-3-chlorophenyl)-3,4-dihydro-2(1H)-quinolinone

The procedure of example 112 was followed here, using 1,4-dibromo-2-chlorobenzene in place of 1,4-dibromo-2-nitrobenzene, to give the title compound (16%) as white solid. ¹H NMR (400 MHz, CDCl₃) δ 7.62 (m, 2H), 7.34 (m, 2H), 7.27 (m, 1H), 6.85 (d, J=7.3 Hz, 1H), 3.01 (t, J=7.1 Hz, 2H), 2.65 (t, J=7.3 Hz, 2H). MS (ES+) m/e 336 [M+H]⁺.

### EXAMPLE 114

### 6-[4-Bromo-3-(trifluoromethyl)phenyl]-3,4-dihydro-2(1H)-quinolinone

The procedure of example 112 was followed here, using 1,4-dibromo-2-(trifluoromethyl)benzene in place of 1,4-dibromo-2-nitrobenzene, to give the title compound (16%) as a white solid. ¹H NMR (400 MHz, CDCl₃) δ 8.25 (br s, 1H), 7.82 (m, 1H), 7.57 (dd, J=2.3, 8.4 Hz, 1H), 7.41 (m, 2H), 6.89 (d, J=8.8 Hz, 1H), 3.09 (t, J=7.4 Hz, 2H), 2.72 (t, J=7.1 Hz, 2H). MS (ES+) m/e 370 [M+H]⁺.

### EXAMPLE 115

### 6-(4-Chlorophenyl)-3,4-dihydro-2(1H)-quinolinone

A mixture of 1-chloro-4-iodobenzene (262 mg, 1.1 mmol), (2-oxo-1,2,3,4-tetrahydro-6-quinolinyl)boronic acid (example 59a, 211 mg, 1.1 mmol), tetrakis(triphenylphosphine)palladium (0) (46 mg, 0.04 mmol) and sodium carbonate (350 mg, 3.3 mmol) in water (1.0 mL) and dioxane (6.0 mL) were sealed under nitrogen in a pressure bottle and stirred at 90° C. overnight. The cooled mixture was filtered through Celite®, extracted into ethyl acetate, dried and evaporated. The residue was separated by preparative HPLC (20-100% acetonitrile-water 0.1% TFA) to give the title compound (17 mg, 6%). ¹H NMR (400 MHz, CDCl₃) δ 7.73 (br s, 1H), 7.48-7.50 (m, 2H), 7.38-7.43 (m, 3H), 6.83 (d, J=8.8 Hz, 1H), 3.06 (t, J=7.2 Hz, 2H), 2.71 (t, J=8 Hz, 2H).

### EXAMPLE 116

### 6-[4-(1H-Indol-1-yl)phenyl]-3,4-dihydro-2(1H)-quinolinone

### a) 6-(4-Aminophenyl)-3,4-dihydro-2(1H)-quinolinone

A mixture of 6-bromo-3,4-dihydro-2(1H)-quinolinone (2.64 g, 11.68 mmol), 4-(4,4,5,5-tetramethyl-1,3,2-dioxaborolan-2-yl)aniline (2.81 g, 12.80 mmol), sodium carbonate (3.7 g, 34.9 mmol) and tetrakis(triphenylphosphine)palladium (0) (250 mg, 0.22 mmol) in water (20 mL) and dioxane (50 mL) was sealed in pressure bottle under argon and heated at 100° C. overnight. The mixture was cooled and diluted with water and extracted twice with ethyl acetate, the combined extracts washed with water and brine, dried and evaporated. The residue was slurried in ether to give the title compound as a pale yellow solid (2.5 g, 90%). ¹H NMR (400 MHz, D₆-DMSO) δ 10.07 (s, 1H), 7.34 (s, 1H), 7.28-7.31 (m, 4H), 6.84 (d, J=8.2 Hz, 1H), 6.60 (dd, J=6.7 and 1.8 Hz, 2H), 5.16 (br s, 2H), 2.90 (t, J=7.1 Hz, 2H), 2.45 (t, J=7.2 Hz, 2H).

### b) 6-[4-(1H-Indol-1-yl)phenyl]-3,4-dihydro-2(1H)-quinolinone

A mixture of 6-(4-aminophenyl)-3,4-dihydro-2(1H)-quinolinone (132 mg, 0.97 mmol) and 2,5-dimethoxytetrahydrofuran (150 uL, 1.17 mmol) in acetic acid (5.0 mL) was heated under reflux for 1 hour. The mixture was evaporated and separated by preparative HPLC (20-100% acetonitrile—water—TFA 0.1%), dissolved in ethyl acetate, washed with sodium hydrogen carbonate solution and filtered through Celite® to give the title compound (30 mg, 9%). ¹H NMR (400 MHz, CDCl₃) δ 8.18 (br s, 1H), 7.70-7.74 (m, 3H), 7.64 (d, J=8.4 Hz, 1H), 6.60 (d, J=8.2 Hz, 2H), 7.48-7.51 (m, 2H), 7.40 (d, J=3.6 Hz, 1H), 7.27 (dd, J=8.4 and 1.6 Hz, 1H), 7.21 (dd, J=8.0 and 1.2 Hz, 1H), 6.90 (d, J=8.8 Hz, 1H), 6.74 (dd, J=3.2 and 1.6 Hz, 1H), 3.10 (t, J=7.2 Hz, 2H), 2.74 (t, J=7.2 Hz, 2H).

### EXAMPLE 117

### 6-(4-Bromo-3-methylphenyl)-3,4-dihydro-2(1H)-quinolinone

The procedure of example 112 was followed here, using 1,4-dibromo-2-methylbenzene in place of 1,4-dibromo-2-nitrobenzene, to give the title compound (13%) as a white solid. ¹H NMR (400 MHz, CDCl₃) δ 7.42 (d, J=8.1 Hz, 1H), 7.27 (d, J=2.3 Hz, 1H), 7.24 (m, 2H), 7.09 (m, 1H), 6.72 (d, J=7.5 Hz, 1H), 2.89 (t, J=7.4 Hz, 2H), 2.52 (t, J=7.8 Hz, 2H), 2.32 (s, 3H). MS (ES+) m/e 316 [M+H]⁺.

### EXAMPLE 118

### 6-(3-Amino-4-bromophenyl-3,4-dihydro-2(1H)-quinolinone

The procedure of example 112 was followed here, using 2,5-dibromoaniline in place of 1,4-dibromo-2-nitrobenzene, to give the title compound (12%) as a white solid. ¹H NMR (400 MHz, d₆-DMSO) δ 10.18 (s, 1H), 7.36 (m, 3H), 7.03 (d, J=2.3 Hz, 1H), 6.90 (d, J=8.3 Hz, 1H), 6.72 (dd, J=2.3, 8.3 Hz, 1H), 2.93 (t, J=7.4 Hz, 2H), 2.48 (t, J=7.1 Hz, 2H). MS (ES+) m/e 317 [M+H]⁺.

### EXAMPLE 119

### 8-(2,2-Difluorobenzo[1,3]dioxol-5-yl)-2,3-dihydro-5H-benzo[b][1,4]thiazepin-4-one

### a) 2,2-Difluoro-5-(3-fluoro-4-nitrophenyl)-1,3-benzodioxole

A slurry of 5-bromo-2,2-difluoro-1,3-benzodioxole (1.23 g, 5.2 mmol), 4-N—BOC-amino-3-fluorophenylboronic acid (1.58 g, 6.2 mmol), [1,1′-bis(diphenylphosphino)ferrocene]dichloropalladium(II)dichloromethane adduct (0.131 g, 0.16 mmol), and 2M aqueous sodium carbonate solution (7.8 mL, 15.6 mmol) in 1,4-dioxane (25.0 mL) was heated at reflux for 2.5 hours then cooled to ambient temperature. The reaction was dried over sodium sulfate then filtered and evaporated under reduced pressure to a residue. Purification by column chromatography on silica (0-65% gradient of ethyl acetate in hexanes) gave 1,1-dimethylethyl [4-(2,2-difluoro-1,3-benzodioxol-5-yl)-2-fluorophenyl]carbamate as a waxy white solid (54%).

A solution of 1,1-dimethylethyl [4-(2,2-difluoro-1,3-benzodioxol-5-yl)-2-fluorophenyl]carbamate (0.992 g, 2.70 mmol) in 1:1 trifluoroacetic acid and dichloromethane (20 mL) was stirred at ambient temperature for 1.5 hours then evaporated under reduced pressure. The resulting residue was treated with glacial acetic acid (20.0 mL) and sodium perborate tetrahydrate (2.08 g, 13.5 mmol) then heated at 75° C. for 3 hours. The reaction was cooled to ambient temperature then poured onto ice (120 g). After 1 hour, the slurry was filtered and the collected solids were rinsed with water then suction dried. Purification of the solids by silica chromatography (0 to 50% gradient of ethyl acetate in hexanes) gave the title compound (0.249 g, 31%) as a yellow solid. MS (ES) m/e 298 (M+H)⁺.

### b) 8-(2,2-Difluorobenzo[1,3]dioxol-5-yl)-2,3-dihydro-5H-benzo[b][1,4]thiazepin-4-one

The procedure of example 55 was followed here, using 2,2-difluoro-5-(3-fluoro-4-nitrophenyl)-1,3-benzodioxole in place of 3-fluoro-4-nitro-4′-(trifluoromethyl)biphenyl, to give the title compound (10%) as a tan solid. MS (ES) m/e 336 (M+H)⁺.

### EXAMPLE 120

### ±2-Oxo-6-[4-(trifluoromethyl)phenyl]-1,2,3,4-tetrahydro-3-quinolinecarboxamide

A solution of +ethyl 2-oxo-6-[4-(trifluoromethyl)phenyl]-1,2,3,4-tetrahydro-3-quinolinecarboxylate (example 104(b), 0.051 g, 0.140 mmol) in 7M methanolic ammonia (10 mL) was heated in a sealed tube at 80° C. for 8 h, then cooled and evaporated to dryness under reduced pressure. The residue was chromatographed (silica gel, 2-10% methanol/dichloromethane) and the product triturated (ether) to give the title compound (0.012 g, 26%) as a solid. ¹H NMR (400 MHz, D₆-DMSO) δ 10.43 (s, 1H), 7.86 (d, J=8.3 Hz, 2H), 7.78 (d, J=8.4 Hz, 2H), 7.62 (s, 1H), 7.56 (dd, J=8.2, 1.9 Hz, 1H), 7.51 (s, 1H), 7.13 (s, 1H), 6.97 (d, J=8.2 Hz, 1H), 3.42 (dd, J=9.4, 6.9 Hz, 1H), 3.20 (dd, J=16.1, 9.4 Hz, 1H), 3.11 (dd, J=16.1, 6.9 Hz, 1H).

### EXAMPLE 121

### 6-[3-(1H-Pyrrol-1-yl)-4-(trifluoromethyl)phenyl]-3,4-dihydro-2(1H)-quinolinone

A mixture of 6-[3-amino-4-(trifluoromethyl)phenyl]-3,4-dihydro-2(1H)-quinolinone (20 mg, 0.065 mmol) and dimethoxytetrahydrofuran (0.1 mL, 0.77 mmol) was heated under reflux in acetic acid (3.0 mL) for 20 minutes. The mixture was partitioned between ethyl acetate and sodium hydrogen carbonate solution and the aqueous was extracted with ethyl acetate. The combined extracts were washed with brine, dried and evaporated. The title compound was obtained as a solid from diethyl ether-hexane (15 mg, 65%). ¹H NMR (400 MHz, D₆-DMSO) δ 10.27 (s, 1H), 7.92 (s, 2H), 7.71 (s, 2H), 7.63 (dd, J=8.1 and 2.0 Hz, 1H), 7.02 (s, 2H), 6.96 (d, J=8.3 Hz, 1H), 6.26 (s, 1H), 2.96 (t, J=7.8 Hz, 2H), 2.50 (t, 2H, under solvent peak).

### EXAMPLE 122

### 6-[4-(Trifluoromethyl)-1-cyclohexen-1-yl]-3,4-dihydro-2(1H)-quinolinone

To a solution of (2-oxo-1,2,3,4-tetrahydro-6-quinolinyl)boronic acid (example 59a, 100 mg, 0.523 mmol) and tetrakis(triphenylphosphine)palladium (0) (30 mg, 0.026 mmol) in 2M aqueous potassium carbonate (2 mL) and dioxane (2 mL) was added 4-(trifluoromethyl)-1-cyclohexen-1-yl trifluoromethanesulfonate (312 mg, 1.047 mmol). The reaction mixture was heated at 105° C. for 2 h. After cooling to room temperature, ethyl acetate (40 mL) was added and the reaction mixture was filtered through a pad of Celite®. The organic layer was separated, washed with saturated sodium bicarbonate (30 mL) and brine (30 mL), dried over MgSO₄, filtered and evaporated down to residue. Purification by rp-HPLC gave the desired product (37 mg, 24%) as a white powder. MS (ES+) m/e 296 [M+H]⁺.

### EXAMPLE 123

### 6-[4-(1-Methylethyl-1-cyclohexen-1-yl]-3,4-dihydro-2(1H)-quinolinone

The title compound was prepared following the procedure described in example 122 except substituting 4-(1-methylethyl)-1-cyclohexen-1-yl trifluoromethanesulfonate for 4-(trifluoromethyl)-1-cyclohexen-1-yl trifluoromethanesulfonate. ¹H NMR (400 MHz, CDCl₃) δ ppm 8.54 (s, 1H) 7.18 (m, 2H) 6.73 (m, 1H) 6.06 (m, 1H) 2.92-3.00 (m, 2H) 2.64 (ddd, J=8.46, 6.69, 3.79 Hz, 2H) 2.35-2.45 (m, 2H) 2.22 (s, 1H) 1.93 (ddd, J=9.79, 4.99, 2.65 Hz, 2H) 1.47-1.58 (m, 1H) 1.29-1.40 (m, 2H) 0.93 (dd, J=6.57, 5.56 Hz, 6H). MS (ES+) m/e 270 [M+H]⁺.

### EXAMPLE 124

### ±6-[3,5-Difluoro-4-(trifluoromethyl)phenyl]-3-hydroxy-3,4-dihydro-2(1H)-quinolinone

5-bromo-1,3-difluoro-2-(trifluoromethyl)benzene (0.090 g, 0.344 mmol), bis(pinacolato)diboron (0.105 g, 0.413 mmol), potassium acetate (0.110 g, 1.12 mmol), in DMF (2.0 mL) were heated to 80° C. for 2.5 hours. The reaction mixture was cooled and ±6-bromo-3-hydroxy-3,4-dihydro-2(1H)-quinolinone (example 85b, 0.10 g, 0.413 mmol), 2M Na₂CO₃ in H₂O (0.97 mL), and [1,1′-bis(diphenylphosphino)ferrocene]dichloropalladium(II)dichloromethane adduct (0.02 g, 0.027 mmol) were added. The reaction was then heated to 80° C. for 12 hours. The mixture was cooled, diluted with ethyl acetate, and washed twice with water and twice with brine, dried over Na₂SO₄, filtered, and concentrated. The crude mixture was purified by reverse-phase HPLC (5%-100% CH₃CN/H₂O; 0.1% TFA, rt=6.49/12 min) to obtain the title compound (0.02 g, 0.058 mmol, 17%) as a white solid. ¹H NMR (400 MHz, CDCl₃) δ 10.37 (bs, 1H), 7.73 (m, 4H), 6.94 (d J=8.0 Hz, 1H), 5.60 (d, 1H), 4.17 (m, 1H), 4.11 (q, 1H), 2.9 (q, 1H). MS (ES+) m/e 344 [M+H]⁺.

### EXAMPLE 125

### ±3-(Hydroxymethyl)-6-[4-(trifluoromethyl)phenyl]-3,4-dihydro-2(1H)-quinolinone

Ethyl chloroformate (0.037 mL, 0.387 mmol) was injected dropwise into a stirred solution of ±2-oxo-6-[4-(trifluoromethyl)phenyl]-1,2,3,4-tetrahydro-3-quinolinecarboxylic acid (example 104, 0.118 g, 0.352 mmol) and triethylamine (0.054 mL, 0.387 mmol) in tetrahydrofuran (5 mL) at room temperature under argon. After 1 h, the mixture was filtered and the filtrate added slowly to a stirred solution of sodium borohydride (0.027 g, 0.704 mmol) in water (5 mL) at room temperature. After stirring 18 h, 0.1M aqueous hydrochloric acid (50 mL) was added and the mixture extracted with ethyl acetate. The extracts were washed with water and brine, dried (MgSO₄) and evaporated to dryness under reduced pressure. The residue was chromatographed (silica gel, 50-100% ethyl acetate/hexane) and the product triturated (ether) to give the title compound (0.016 g, 14%) as a cream solid. ¹H NMR (400 MHz, D₆-DMSO) δ 10.26 (s, 1H), 7.87 (d, J=8.3 Hz, 2H), 7.78 (d, J=8.4 Hz, 2H), 7.62 (s, 1H), 7.55 (dd, J=8.2, 2.0 Hz, 1H), 6.97 (d, J=8.2 Hz, 1H), 4.75 (t, J=5.4 Hz, 1H), 3.73 (m, 1H), 3.58 (m, 1H), 3.08 (dd, J=16.0, 6.2 Hz, 1H), 2.93 (dd, J=15.9, 10.5 Hz, 1H), 2.59 (m, 1H).

### EXAMPLE 126

### 7-(2,2-Difluorobenzo[1,3]dioxol-5-yl)-4H-benzo[1,4]oxazin-3-one

A solution of 2,2-difluoro-5-(3-fluoro-4-nitrophenyl)-1,3-benzodioxole (0.419 g, 1.41 mmol) and ethyl glycolate (0.221 g, 2.12 mmol) in anhydrous N,N-dimethylformamide (10.0 mL) was treated with 60% sodium hydride in mineral oil (0.099 g, 2.47 mmol) in one portion. The resulting red reaction was stirred for 2 hours then diluted with water (50 mL) and extracted into dichloromethane. The extracts were washed with saturated aqueous sodium bicarbonate then dried over magnesium sulfate. Evaporation under reduced pressure gave the crude product as a dark oil. This oil was taken into glacial acetic acid (25.0 mL) then treated with zinc powder (300 mg, 4.59 mmol). After stirring at ambient temperature for 24 hours, the reaction was filtered and the filtrate was evaporated under reduced pressure to a gum that was purified by preparative HPLC. The product was allowed to precipitate from the HPLC fractions overnight then was filtered, rinsed with water and vacuum dried to give the title compound as light tan plates. MS (ES) m/e 306 (M+H)⁺.

### EXAMPLE 127

### 4-(2-Oxo-1,2,3,4-tetrahydro-6-quinolinyl)benzonitrile

A mixture of 4-bromobenzonitrile (115 mg, 0.63 mmol), (2-oxo-1,2,3,4-tetrahydro-6-quinolinyl)boronic acid (150 mg, 0.63 mmol), tetrakis(triphenylphosphine)palladium (0) (30 mg, 0.025 mmol) and sodium carbonate (200 mg, 1.88 mmol) in water (3.0 mL) and dioxan (10.0 mL) was sealed under nitrogen in a pressure bottle and stirred at 10° C. overnight. The mixture was evaporated onto silica gel and chromatographed (silica gel, hexane to ethyl acetate) to give the title compound as a white solid (85 mg, 55%). ¹H NMR (400 MHz, D₆-DMSO) δ 10.27 (s, 1H), 7.99 (d, J=6.6 Hz, 2H), 7.84 (d, J=6.6 Hz, 2H), 7.62 (d, J=2.0 Hz, 1H), 7.57 (dd, J=8.3 and 2.2 Hz, 1H), 6.96 (d, J=8.0 Hz, 1H), 2.96 (t, J=7.3 Hz, 2H), 2.49 (t, 2H, under solvent peak).

### EXAMPLE 128

### 2-Fluoro-4-(2-oxo-1,2,3,4-tetrahydro-6-quinolinyl)benzonitrile

A mixture of 4-bromo-2-fluorobenzonitrile (126 mg, 0.63 mmol), (2-oxo-1,2,3,4-tetrahydro-6-quinolinyl)boronic acid (150 mg, 0.63 mmol), tetrakis(triphenylphosphine)palladium (0) (30 mg, 0.025 mmol) and sodium carbonate (200 mg, 1.88 mmol) in water (3.0 mL) and dioxane (10.0 mL) was sealed under nitrogen in a pressure bottle and stirred at 100° C. overnight. The mixture was evaporated onto silica gel and chromatographed (silica gel, hexane to ethyl acetate) to give the title compound as a white solid (70 mg, 42%). ¹H NMR (400 MHz, D₆-DMSO) δ 10.31 (s, 1H), 7.96 (t, J=8.0 Hz, 1H), 7.85 (d, J=11.3 Hz, 1H), 7.70-7.74 (m, 2H), 7.64 (d, J=8.3 Hz, 1H), 6.96 (d, J=8.1 Hz, 1H), 2.96 (t, J=7.0 Hz, 2H), 2.49 (t, 2H, under solvent peak).

### EXAMPLE 129

### ±3-Hydroxy-6-{4-[(trifluoromethyl)sulfonyl]phenyl}-3,4-dihydro-2(1H)-quinolinone

1-Bromo-4-[(trifluoromethyl)sulfonyl]benzene (PCT Int. Appl., 1994, WO9406783A1, 0.083 g, 0.338 mmol), bis(pinacolato)diboron (0.0944 g, 0.372 mmol), potassium acetate (0.0995 g, 1.01 mmol), in DMF (2.0 mL) was heated to 80° C. for 2.5 hours and then cooled and ±6-bromo-3-hydroxy-3,4-dihydro-2(1H)-quinolinone (example 85b, 0.090 g, 0.371 mmol), 2M Na₂CO₃ in H₂O (0.97 mL), and dichloro[1,1′-bis(diphenylphosphino)ferrocene]palladium(II)dichloromethane adduct (0.02 g, 0.027 mmol) were added. The reaction was then heated to 80° C. After 12 hours the reaction mixture was cooled, diluted with ethyl acetate, and washed twice with water and twice with brine, dried over Na₂SO₄, filtered, and concentrated. The crude mixture was purified by reverse-phase HPLC (5%-100% CH₃CN/H₂O; 0.1% TFA) to obtain the title compound (0.029 g, 23%) as a white solid. ¹H NMR (400 MHz, D₆-DMSO) δ 10.39 (s, 1H), 8.16 (d, J=8.6 Hz, 2H), 8.10 (d, J=8.7 Hz, 2H), 7.73 (s, 1H), 7.67 (dd, J=2.0, 6.2 Hz, 1H), 7.00 (d, J=8.3 Hz, 1H), 5.60 (d, 1H), 4.19 (m, 1H), 3.15 (dd, 1H), 2.95 (dd, 1H).

### EXAMPLE 130

### ±6-[3-Fluoro-4-(trifluoromethyl)phenyl]-3-hydroxy-3,4-dihydro-2(1H)-quinolinone

4-Bromo-2-fluoro-1-(trifluoromethyl)benzene (0.091 mL, 0.375 mmol), bis(pinacolato)diboron (0.10 g, 0.394 mmol), potassium acetate (0.11 g, 1.12 mmol), dichloro[1,1′-bis(diphenylphosphino)ferrocene]palladium(II)dichloromethane adduct (0.020 g, 0.027 mmol) in DMF (2.0 mL) were heated to 80° C. under nitrogen for 2.5 hours. After the reaction was cooled, dichloro[1′,1′-bis(diphenylphosphino)ferrocene]palladium(II)dichloromethane adduct (0.020 g, 0.027 mmol) was added followed by addition of ±6-bromo-3-hydroxy-3,4-dihydro-2(1H)-quinolinone (example 85b, 0.095 g, 0.392 mmol) and 2M Na₂CO₃ in water (0.98 mL). The reaction was heated to 80° C. After 12 hours the reaction mixture was cooled and diluted with ethyl acetate, then washed with water and brine, dried over Na₂SO₄, filtered, and concentrated under reduced pressure. The crude reaction mixture was purified using reverse-phase preparative HPLC (10% CH3CN/H2O to 100% CH3CN/H2O with 0.1% TFA) to give the title compound as a solid. ¹H NMR (400 MHz, D₆-DMSO) δ 10.34 (bs, 1H), 7.79 (m, 2H), 7.69 (m, 2H), 7.59 (d, J=8.6 Hz, 1H), 6.96 (d, J=8.3 Hz, 1H), 5.58 (bs, 1H), 4.18 (m, 1H), 3.13 (dd, 1H), 2.93 (dd, 1H).

### EXAMPLE 131

### Monopolar Spindle Formation Following Application of a KSP Inhibitor of the Invention

Human tumor cells Skov-3 (ovarian) were plated in 96-well plates at densities of 4,000 cells per well, allowed to adhere for 24 hours, and treated with various concentrations of test compounds of the present invention for 24 hours. Cells were fixed in 4% formaldehyde and stained with antitubulin antibodies (subsequently recognized using fluorescently-labeled secondary antibody) and Hoechst dye (which stains DNA).

Compounds of this class were found by visual inspection to cause cell cycle arrest in the prometaphase stage of mitosis, although results varied. Where cell cycle arrest was evident, DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles. Although most of the compounds that inhibited KSP activity biochemically did exhibit cell cycle arrest, for some, cell cycle arrest was not detected.

### EXAMPLE 132

### Inhibition of Cellular Proliferation in Tumor Cell Lines Treated with KSP Inhibitors of the Invention

Cells were plated in 96-well plates at densities from 1000-2500 cells/well of a 96-well plate and allowed to adhere/grow for 24 hours. They were then treated with various concentrations of compounds of the present invention for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CellTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this. Compounds of this class were found to inhibit cell proliferation in human ovarian tumor cell lines (SKOV-3), although results varied. Although most of the compounds that inhibited KSP activity biochemically did inhibit cell proliferation, for some compounds inhibition was relatively low or not detected.

A GI₅₀ was calculated by plotting the concentration of compound in μM vs the percentage of cell growth in treated wells. The GI₅₀ calculated for the compounds is the estimated concentration at which growth is inhibited by 50% compared to control, i.e., the concentration at which:

100×[(Treated₄₈−T₀)/(Control₄₈−T₀)]=50.

All concentrations of compounds are tested in duplicate and controls are averaged over 12 wells. A very similar 96-well plate layout and GI₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative methods.

Compounds of this class were found to inhibit cell proliferation, although GI₅₀ values varied. GI₅₀ values for the compounds tested ranged from about <78 nM to greater than the highest concentration tested. By this we mean that although most of the compounds that inhibited KSP activity biochemically did inhibit cell proliferation, for some, at the highest concentration tested (generally about 20 μM), cell growth was inhibited less than 50%. Many of the compounds have GI₅₀ values less than 10 μM, and many have GI₅₀ values less than 1 μM. Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful. However, preferably, compounds will have GI₅₀ values of less than 1 mM. More preferably, compounds will have GI₅₀ values of less than 20 μM. Even more preferably, compounds will have GI₅₀ values of less than 10 μM. Further reduction in GI₅₀ values may also be desirable, including compounds with GI₅₀ values of less than 1 μM. Some of the compounds of the invention inhibit cell proliferation with GI₅₀ values below 200 nM.

### EXAMPLE 133

### Calculation of IC₅₀

Measurement of a compound's IC₅₀ for KSP activity uses an ATPase assay. The following solutions are used: Solution 1 consists of 2 mM phosphoenolpyruvate potassium salt (Sigma P-7127), 0.03-1 mM ATP (Sigma A-3377), 1 mM DTT (Sigma D-9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT400301), and 1 mM EGTA (Sigma E3889). Solution 2 consists of 0.6 mM NADH (Sigma N8129), 0.2 mg/mL BSA (Sigma A7906), pyruvate kinase 7 U/mL, L-lactate dehydrogenase 10 U/mL (Sigma P0294), 50-100 nM KSP motor domain, 200 μg/mL microtubules, 1 mM DTT (Sigma D9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT4003-01), and 1 mM EGTA (Sigma E3889). Serial dilutions (8-12 two-fold dilutions) of the compound are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x the compound concentration.

The compounds of the invention exhibited a KSP IC₅₀ of 10 μM or less using an ATP concentration of 15 μM.

This application (including the description and claims) may be used as a basis for priority in respect of any subsequent application or claims. The claims of this or such subsequent application may be directed to any feature or combination of features described herein. They may take the form of product, composition, process, or use claims and may include, by way of example and without limitation the following claims.

We claim:

1. A compound of Formula (I): 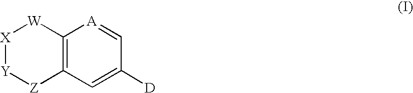
where
W is NR¹, O, CH₂, or CH(OH);
R¹ is H, C₁₋₄alkyl, C₁₋₄alkylaryl, CO₂Bu^{t}, CO₁₋₄alkyl, CH₂CONMe₂, or CO₂CH₂Ph;
X is C═O, C═S, C═NOH, SO₂, CH₂, or CH(OH), provided that, when W is CH(OH), X is not SO₂;
Y-Z is V—CHR²; where
V is O, NR³, or CHR⁴;
R² is H or C₁₋₄alkyl;
R³ is H, C₁₋₂alkylOH, or C₁₋₂alkyl; and
R⁴ is H, C₁₋₄alkyl, COSEt, NH₂, OH, NHCHO, NHCOC₁₋₄alkyl, NHSO₂C₁₋₄alkyl, CO₂H, CH₂OH, or CONH₂;
or Y-Z is V²═CR⁵, where
V² is N or CH; and
R⁵ is H, Me or NH₂;
or Y-Z is V³—U, where
V³ is CMe₂, CO or CHR⁴;
U is NR⁷, O, S, or SO₂;
R⁷ is H, CHO, or CH₂R⁸, and
R⁸ is H, CN, CO₂Me, CONH₂, CO₂H, or CH₂OH;
or Y-Z is CH═N;
A is N or CR¹⁰;
R¹⁰ is H, F, CO₂H, NH₂, or NO₂;
D is selected from: 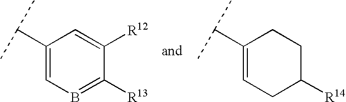
B is N or CR¹¹;
R¹¹ is H or F;
R¹² is H, halogen, Me, NH₂, NHAc, NO₂, CF₃, 1-pyrryl, or CH₂CN;
R¹³ is H, CF₃, CN, SO₂CF₃, SO₂NMe₂, SO₂C₁₋₃alkyl, SC₁₋₃alkyl, halogen, 1-indolyl, Prⁱ, Bu^{t}, NMe₂, or NO₂;
or R¹² and R¹³ taken together are OCF₂O;
provided that, when R¹³ is H, B and R¹² are CH and CF₃ respectively or CF and F respectively; and
R¹⁴ is CF₃ or C₂₋₅alkyl;
or a pharmaceutically acceptable derivative or solvate thereof.

2. A compound of claim 1 wherein W is NR¹ and R¹ is H.

3. A compound of claim 2 wherein:
X is CO, CS, or SO₂;
Y-Z is V—CHR² wherein
V is O, NR³ and R³ is H, or CHR⁴ and R⁴ is H, OH, or CO₂H;
Z is CHR² and R² is H;
A is CR¹⁰ and R¹⁰ is H or F, or A is N;
D is 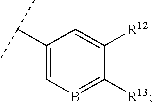
B is CR¹¹;
R¹² is H, F, Cl, CF₃, 1-pyrryl, or NO₂;
R¹³ is CF₃, Cl, Br, SO₂CF₃, Prⁱ, or Bu^{t}; or
R¹² and R¹³ together are OCF₂O.

4. A compound of claim 3 wherein X is C═O.

5. A compound of claim 4 wherein
V is CHR⁴ and R⁴ is H, OH, or CO₂H;
R¹² is H, F, CF₃, or NO₂;
R¹³ is CF₃ or SO₂CF₃;
or R¹² and R¹³ together are OCF₂O.

6. A compound of claim 2 wherein:
X is CO, CS, or SO₂;
Y-Z is V³—U wherein
V³ is CMe₂, CO or CHR⁴ and R⁴ is H or CO₂H;
U is O, S, or NR⁷ and R⁷ is H;
A is CR¹⁰ and R¹⁰ is H or F, or A is N;
D is 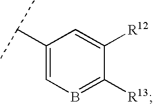
B is CR¹¹;
R¹² is H, F, Cl, CF₃, 1-pyrryl, or NO₂;
R¹³ is CF₃, Cl, Br, SO₂CF₃, Prⁱ, or Bu^{t}; or
R¹² and R¹³ together are OCF₂O.

7. A compound of claim 6 wherein X is C═O.

8. A compound of claim 7 wherein:
V³ is CHR⁴ and R⁴ is H or CO₂H;
U is S;
R¹² is H, F, CF₃, or NO₂;
R¹³ is CF₃ or SO₂CF₃;
or R¹² and R¹³ together are OCF₂O.

9. A compound of Formula (II): 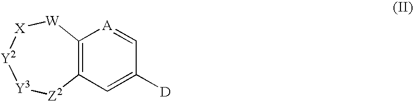
where
W is NR¹, O, CH₂, or CH(OH);
R¹ is H, C₁₋₄alkyl, C₁₋₄alkylaryl, CO₂Bu^{t}, CO₁₋₄alkyl, CH₂CONMe₂, or CO₂CH₂Ph;
X is C═O, C═S, C═NOH, SO₂, CH₂, or CH(OH), provided that, when W is CH(OH), X is not SO₂;
Y² is O, NR³, CHR⁴, or CMe₂;
Y³ is CH₂, O, S, or NH;
Z² is CHR², NR⁷, O, S, or SO₂;
or Y³-Z² taken together is N═CH when Y² is CHR⁴;
provided that, when Y² is O or NR³, Y³ is CH₂ and Z² is CHR²; and
provided that, when Y³ is O, S, or NH, Z² is CHR² and Y² is CHR⁴; and
provided that, when Z² is NR⁷, O, S, or SO₂, Y² is CHR⁴, or CMe₂ and Y³ is CH₂;
R² is H or C₁₋₄alkyl;
R³ is H, C₁₋₂alkylOH, or C₁₋₂alkyl;
R⁴ is H, C₁₋₄alkyl, COSEt, NH₂, OH, NHCHO, NHCOC₁₋₄alkyl, NHSO₂C₁₋₄alkyl, CO₂H, CH₂OH, or CONH₂;
R⁷ is H, CHO, or CH₂R⁸;
R⁸ is H, CN, CO₂Me, CONH₂, CO₂H, or CH₂OH;
A is N or CR¹⁰;
R¹⁰ is H, F, CO₂H, NH₂, or NO₂;
D is selected from: 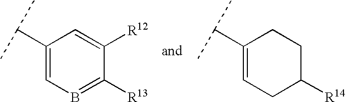
B is N or CR¹¹;
R¹¹ is H or F;
R¹² is H, halogen, Me, NH₂, NHAc, NO₂, CF₃, 1-pyrryl, or CH₂CN;
R¹³ is H, CF₃, CN, SO₂CF₃, SO₂NMe₂, SO₂C₁₋₃alkyl, SC₁₋₃alkyl, halogen, 1-indolyl, Prⁱ, Bu^{t}, NMe₂, or NO₂;
or R¹² and R¹³ taken together are OCF₂O;
provided that, when R¹³ is H, B and R¹² are CH and CF₃ respectively or CF and F respectively; and
R¹⁴ is CF₃ or C₂₋₅alkyl;
or a pharmaceutically acceptable derivative or solvate thereof.

10. A compound of claim 9 wherein W is NR¹ and R¹ is H.

11. A compound of claim 10 wherein:
X is CO, CS, or SO₂;
Y² is CHR⁴ and R⁴ is H, OH, or CO₂H;
Z² is CHR² and R² is H, or Z is O or S;
A is CR¹⁰ and R¹⁰ is H or F, or A is N;
D is 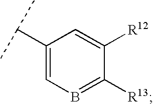
B is CR¹¹;
R¹² is H, F, Cl, CF₃, 1-pyrryl, or NO₂;
R¹³ is CF₃, Cl, Br, SO₂CF₃, Prⁱ, or Bu^{t};
or R¹² and R¹³ together are OCF₂O.

12. A compound of claim 11 wherein X is C═O and Y² is CH₂.

13. A compound of claim 12 wherein:
R¹² is H, F, CF₃, or NO₂;
R¹³ is CF₃ or SO₂CF₃;
or R¹² and R¹³ together are OCF₂O.

14. A compound of Formula (III): 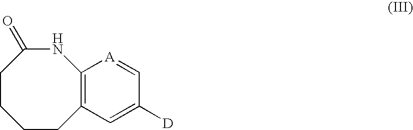
where
A is N or CR¹⁰;
R¹⁰ is H, F, CO₂H, NH₂, or NO₂;
D is selected from: 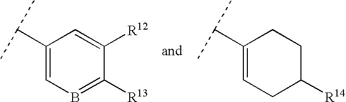
B is N or CR¹¹;
R¹¹ is H or F;
R¹² is H, halogen, Me, NH₂, NHAc, NO₂, CF₃, 1-pyrryl, or CH₂CN;
R¹³ is H, CF₃, CN, SO₂CF₃, SO₂NMe₂, SO₂C₁₋₃alkyl, SC₁₋₃alkyl, halogen, 1-indolyl, Prⁱ, Bu^{t}, NMe₂, or NO₂;
or R¹² and R¹³ taken together are OCF₂O;
provided that, when R¹³ is H, B and R¹² are CH and CF₃ respectively or CF and F respectively; and
R¹⁴ is CF₃ or C₂₋₅alkyl;
or a pharmaceutically acceptable derivative or solvate thereof.

15. A compound selected from:
6-[4-(trifluoromethyl)phenyl]-3,4-dihydro-1H-2,1,3-benzothiadiazine 2,2-dioxide
6-[4-(trifluoromethyl)phenyl]-1H-2,1,3-benzothiadiazine 2,2-dioxide
6-[4-(trifluoromethyl)phenyl]-3,4-dihydro-2(1H)-quinazolinone
1-(phenylmethyl)-6-[4-(trifluoromethyl)phenyl]-3,4-dihydro-2(1H)-quinazolinone
6-[4-(trifluoromethyl)phenyl]-3,4-dihydro-2(1H)-quinazolinethione
6-[4-(trifluoromethyl)phenyl]-1,4-dihydro-2H-3,1-benzoxazin-2-one
4-methyl-6-[4-(trifluoromethyl)phenyl]-3,4-dihydro-2(1H)-quinazolinone
7-[4-(Trifluoromethyl)phenyl]-3,4-dihydro-2(1H)-quinoxalinone
4-methyl-6-[4-(trifluoromethyl)phenyl]-2(1H)-quinazolinone
4-ethyl-6-[4-(trifluoromethyl)phenyl]-3,4-dihydro-2(1H)-quinazolinone
8-fluoro-6-[4-(trifluoromethyl)phenyl]-3,4-dihydro-2(1H)-quinazolinethione
6-(4-Trifluoromethyl-phenyl)-3,4-dihydro-1H-quinolin-2-one
6-[4-(Trifluoromethyl)phenyl]-3,4-dihydro-2(1H)-quinoxalinone
6-[4-(Trifluoromethyl)phenyl]-3,4-dihydro-2(1H)-quinolinethione
7-[4-(trifluoromethyl)phenyl]-2H-1,4-benzothiazin-3(4H)-one
7-[4-(trifluoromethyl)phenyl]-2H-1,4-benzoxazin-3(4H)-one
6-[4-(Trifluoromethyl)phenyl]-2(1H)-quinolinone
6-[4-(Trifluoromethyl)phenyl]-2(1H)-quinoxalinone
7-[4-(trifluoromethyl)phenyl]-2H-1,4-benzothiazin-3(4H)-one 1,1-dioxide
3-Oxo-7-[4-(trifluoromethyl)phenyl]-3,4-dihydro-1(2H)-quinoxalinecarbaldehyde
[3-Oxo-7-[4-(trifluoromethyl)phenyl]-3,4-dihydro-1(2H)-quinoxalinyl]acetonitrile
Methyl [3-oxo-7-[4-(trifluoromethyl)phenyl]-3,4-dihydro-1(2H)-quinoxalinyl]acetate
6-[4-(trifluoromethyl)phenyl]-3,4-dihydro-1,8-naphthyridin-2(1H)-one
[3-Oxo-7-[4-(trifluoromethyl)phenyl]-3,4-dihydro-1(2H)-quinoxalinyl]acetic acid
1-methyl-6-[4-(trifluoromethyl)phenyl]-3,4-dihydro-2(1H)-quinolinone
7-[4-(trifluoromethyl)phenyl]-1,3,4,5-tetrahydro-2H-1-benzazepin-2-one
3-(2-hydroxyethyl)-6-[4-(trifluoromethyl)phenyl]-3,4-dihydro-2(1H)-quinazolinone
6-[4-(Trifluoromethyl)phenyl]-1,2,3,4-tetrahydroquinoline
1,1-dimethylethyl 2-oxo-6-[4-(trifluoromethyl)phenyl]-3,4-dihydro-1(2H)-quinolinecarboxylate
S-ethyl 2-oxo-6-[4-(trifluoromethyl)phenyl]-1,2,3,4-tetrahydro-3-quinolinecarbothioate
6-[4-(trifluoromethyl)phenyl]-1H-4,2,1-benzoxathiazine 2,2-dioxide
6-[4-(trifluoromethyl)phenyl]-3,4-dihydro-2(1H)-naphthalenone
6-[4-(Trifluoromethyl)phenyl]-3,4-dihydro-1H-2,1-benzothiazine 2,2-dioxide
7-[4-(trifluoromethyl)phenyl]-2H-pyrido[3,2-b][1,4]oxazin-3(4H)-one
2-[3-oxo-7-[4-(trifluoromethyl)phenyl]-3,4-dihydro-1(2H)-quinoxalinyl]acetamide
7-[3-(trifluoromethyl)phenyl]-2H-1,4-benzothiazin-3(4H)-one
3,3-Dimethyl-6-[4-(trifluoromethyl)phenyl]-3,4-dihydro-2(1H)-quinoxalinone
1,1-dimethylethyl 3-oxo-7-[4-(trifluoromethyl)phenyl]-2,3-dihydro-4H-1,4-benzothiazine-4-carboxylate
6-(4-Fluoro-3-methyl-phenyl)-3,4-dihydro-1H-quinolin-2-one
4-(2-hydroxyethyl)-6-[4-(trifluoromethyl)phenyl]-3,4-dihydro-2(1H)-quinoxalinone
4-methyl-6-[4-(trifluoromethyl)phenyl]-3,4-dihydro-2(1H)-quinoxalinone
6-[4-(trifluoromethyl)phenyl]-3,4-dihydro-2(1H)-naphthalenone oxime
8-fluoro-6-[4-(trifluoromethyl)phenyl]-2(1H)-quinolinone
8-fluoro-6-[4-(trifluoromethyl)phenyl]-3,4-dihydro-2(1H)-quinolinone
6-(3,5-Difluoro-phenyl)-3,4-dihydro-1H-quinolin-2-one
6-(2,2-Difluoro-benzo[1,3]dioxol-5-yl)-3,4-dihydro-1H-quinolin-2-one
7-[4-(trifluoromethyl)phenyl]-1,3,4,5-tetrahydro-2H-1,4-benzodiazepin-2-one
6-(3-Fluoro-4-(trifluoromethyl)phenyl)-3,4-dihydro-1H-quinolin-2-one
7-(2,2-Difluoro-benzo[1,3]dioxol-5-yl)-1,3,4,5-tetrahydro-benzo[b]azepin-2-one
6-(4-Isopropyl-phenyl)-3,4-dihydro-1H-quinolin-2-one
6-(4-Trifluoromethanesulfonyl-phenyl)-3,4-dihydro-1H-quinolin-2-one
±6-[4-(trifluoromethyl)phenyl]-1,2,3,4-tetrahydro-2-naphthalenol
7-(2,2-Difluorobenzo[1,3]dioxol-5-yl)-4H-benzo[1,4]thiazin-3-one
3-methyl-6-[4-(trifluoromethyl)phenyl]-3,4-dihydro-2(1H)-quinazolinone
8-[4-(trifluoromethyl)phenyl]-2,3-dihydro-1,5-benzothiazepin-4(5H)-one
7-(4-Trifluoromethanesulfonyl-phenyl)-4H-benzo[1,4]thiazin-3-one
7-(3-Fluoro-4(trifluoromethyl)phenyl)-4H-benzo[1,4]thiazin-3-one
8-fluoro-6-[3-fluoro-4-(trifluoromethyl)phenyl]-3,4-dihydro-2(1H)-quinolinone
6-[3-nitro-4-(trifluoromethyl)phenyl]-3,4-dihydro-2(1H)-quinolinone
6-[3-chloro-4-(trifluoromethyl)phenyl]-3,4-dihydro-2(1H)-quinolinone
6-[6-(trifluoromethyl)-3-pyridinyl]-3,4-dihydro-2(1H)-quinolinone
N,N-dimethyl-4-(2-oxo-1,2,3,4-tetrahydro-6-quinolinyl)benzenesulfonamide
6-[4-(dimethylamino)phenyl]-3,4-dihydro-2(1H)-quinolinone
6-(4-tert-Butyl-phenyl)-3,4-dihydro-1H-quinolin-2-one
6-{4-[(1-methylethyl)thio]phenyl}-3,4-dihydro-2(1H)-quinolinone
6-[4-(trifluoromethyl)phenyl]-3,4-dihydro-2H-chromen-2-one
7-[4-(trifluoromethyl)phenyl]-1,3,4,5-tetrahydro-2H-1,3-benzodiazepin-2-one
6-[3-amino-4-(trifluoromethyl)phenyl]-3,4-dihydro-2(1H)-quinolinone
8-amino-6-[4-(trifluoromethyl)phenyl]-3,4-dihydro-2(1H)-quinolinone
7-(4-Trifluoromethanesulfonyl-phenyl)-1,3,4,5-tetrahydro-benzo[b]azepin-2-one
6-[4-(methylsulfonyl)phenyl]-3,4-dihydro-2(1H)-quinolinone
6-(4-nitrophenyl)-3,4-dihydro-2(1H)-quinolinone
4-Amino-6-[4-(trifluoromethyl)phenyl]-2(1H)-quinolinone
6-{4-[(1-methylethyl)sulfonyl]phenyl}-3,4-dihydro-2(1H)-quinolinone
6-[4-(trifluoromethyl)phenyl]-1,2,3,4-tetrahydro-1-naphthalenol
7-(3-Fluoro-4(trifluoromethyl)phenyl)-1,3,4,5-tetrahydro-benzo[b]azepin-2-one
8-nitro-6-[4-(trifluoromethyl)phenyl]-3,4-dihydro-2(1H)-quinolinone
7-[4-(trifluoromethyl)phenyl]-1,5-dihydro-4,1-benzothiazepin-2(3H)-one
3-amino-8-[4-(trifluoromethyl)phenyl]-2,3-dihydro-1,5-benzothiazepin-4(5H)-one trifluoroacetate
6-(4-bromophenyl)-3,4-dihydro-2(1H)-quinolinone
1-(3-methylbutanoyl)-6-[4-(trifluoromethyl)phenyl]-3,4-dihydro-2(1H)-quinolinone
N,N-dimethyl-2-[2-oxo-6-[4-(trifluoromethyl)phenyl]-3,4-dihydro-1(2H)-quinolinyl]acetamide
(3S)-3-amino-8-[4-(trifluoromethyl)phenyl]-2,3-dihydro-1,5-benzoxazepin-4(5H)-one trifluoroacetate
8-[4-(trifluoromethyl)phenyl]-3,4,5,6-tetrahydro-1-benzazocin-2(1H)-one
±3-Hydroxy-6-[4-(trifluoromethyl)phenyl]-3,4-dihydro-2(1H)-quinolinone
7-[4-(trifluoromethyl)phenyl]-1,3,4,5-tetrahydro-2H-1,5-benzodiazepin-2-one
1,1-dimethylethyl 4-oxo-8-[4-(trifluoromethyl)phenyl]-3,4-dihydro-1,5-benzothiazepine-5(2H)-carboxylate
N-[5-(2-oxo-1,2,3,4-tetrahydro-6-quinolinyl)-2-(trifluoromethyl)phenyl]acetamide
N-{(3S)-4-oxo-8-[4-(trifluoromethyl)phenyl]-2,3,4,5-tetrahydro-1,5-benzoxazepin-3-yl}acetamide
N-{(3S)-4-oxo-8-[4-(trifluoromethyl)phenyl]-2,3,4,5-tetrahydro-1,5-benzoxazepin-3-yl}methanesulfonamide
1-acetyl-6-[4-(trifluoromethyl)phenyl]-3,4-dihydro-2(1H)-quinolinone
7-[4-(trifluoromethyl)phenyl]-1,5-dihydro-4,1-benzoxazepin-2(3H)-one
phenylmethyl 2-oxo-6-[4-(trifluoromethyl)phenyl]-3,4-dihydro-1(2H)-quinolinecarboxylate
5-(3-methylbutanoyl)-8-[4-(trifluoromethyl)phenyl]-2,3-dihydro-1,5-benzothiazepin-4(5H)-one
6-[3,5-difluoro-4-(trifluoromethyl)phenyl]-3,4-dihydro-2(1H)-quinolinone
3-methyl-7-[4-(trifluoromethyl)phenyl]-1,3,4,5-tetrahydro-2H-1,5-benzodiazepin-2-one
(3R)-3-amino-8-[4-(trifluoromethyl)phenyl]-2,3-dihydro-1,5-benzoxazepin-4(5H)-one trifluoroacetate
7-[4-(Trifluoromethyl)phenyl]-1,3-dihydro-2H-1,4-benzodiazepin-2-one
8-[4-(trifluoromethyl)phenyl]-2,3-dihydro-1,5-benzoxazepin-4(5H)-one
8-[4-(trifluoromethyl)phenyl]-2,3-dihydro-1,5-benzothiazepin-4(5H)-one 1,1-dioxide
8-amino-6-[3-fluoro-4-(trifluoromethyl)phenyl]-3,4-dihydro-2(1H)-quinolinone
6-[4-(1,1-dimethylpropyl)-1-cyclohexen-1-yl]-3,4-dihydro-2(1H)-quinolinone
[5-(2-oxo-1,2,3,4-tetrahydro-6-quinolinyl)-2-(trifluoromethyl)phenyl]acetonitrile
2-Oxo-6-[4-(trifluoromethyl)phenyl]-1,2,3,4-tetrahydro-3-quinolinecarboxylic acid
(3S)-3-methyl-7-[4-(trifluoromethyl)phenyl]-1,3,4,5-tetrahydro-2H-1,4-benzodiazepin-2-one
2-Oxo-6-[4-(trifluoromethyl)phenyl]-1,2,3,4-tetrahydro-8-quinolinecarboxylic acid
2-{4-oxo-8-[4-(trifluoromethyl)phenyl]-2,3,4,5-tetrahydro-1H-1,5-benzodiazepin-1-yl}acetamide
(+)-3-Hydroxy-6-[4-(trifluoromethyl)phenyl]-3,4-dihydro-2(1H)-quinolinone
3-Hydroxy-6-[4-(trifluoromethyl)phenyl]-3,4-dihydro-2(1H)-quinolinone
6-[4-(1,1-dimethylethyl)-1-cyclohexen-1-yl]-3,4-dihydro-2(1H)-quinolinone
6-(4-ethyl-1-cyclohexen-1-yl)-3,4-dihydro-2(1H)-quinolinone
6-(4-bromo-3-nitrophenyl)-3,4-dihydro-2(1H)-quinolinone
6-(4-bromo-3-chlorophenyl)-3,4-dihydro-2(1H)-quinolinone
6-[4-bromo-3-(trifluoromethyl)phenyl]-3,4-dihydro-2(1H)-quinolinone
6-(4-chlorophenyl)-3,4-dihydro-2(1H)-quinolinone
6-[4-(1H-indol-1-yl)phenyl]-3,4-dihydro-2(1H)-quinolinone
6-(4-bromo-3-methylphenyl)-3,4-dihydro-2(1H)-quinolinone
6-(3-amino-4-bromophenyl)-3,4-dihydro-2(1H)-quinolinone
8-(2,2-Difluoro-benzo[1,3]dioxol-5-yl)-2,3-dihydro-5H-benzo[b][1,4]thiazepin-4-one
2-Oxo-6-[4-(trifluoromethyl)phenyl]-1,2,3,4-tetrahydro-3-quinolinecarboxamide
6-[3-(1H-pyrrol-1-yl)-4-(trifluoromethyl)phenyl]-3,4-dihydro-2(1H)-quinolinone
6-[4-(trifluoromethyl)-1-cyclohexen-1-yl]-3,4-dihydro-2(1H)-quinolinone
6-[4-(1-methylethyl)-1-cyclohexen-1-yl]-3,4-dihydro-2(1H)-quinolinone
±6-[3,5-difluoro-4-(trifluoromethyl)phenyl]-3-hydroxy-3,4-dihydro-2(1H)-quinolinone
±3-(Hydroxymethyl)-6-[4-(trifluoromethyl)phenyl]-3,4-dihydro-2(1H)-quinolinone
7-(2,2-Difluoro-benzo[1,3]dioxol-5-yl)-4H-benzo[1,4]oxazin-3-one
4-(2-oxo-1,2,3,4-tetrahydro-6-quinolinyl)benzonitrile
2-fluoro-4-(2-oxo-1,2,3,4-tetrahydro-6-quinolinyl)benzonitrile
±3-hydroxy-6-{4-[(trifluoromethyl)sulfonyl]phenyl}-3,4-dihydro-2(1H)-quinolinone
±6-[3-fluoro-4-(trifluoromethyl)phenyl]-3-hydroxy-3,4-dihydro-2(1H)-quinolinone;
and pharmaceutically acceptable derivatives and solvates thereof.

16. A compound according to claim 15, selected from:
6-(4-Trifluoromethyl-phenyl)-3,4-dihydro-1H-quinolin-2-one
7-[4-(trifluoromethyl)phenyl]-2H-1,4-benzothiazin-3(4H)-one
7-[4-(trifluoromethyl)phenyl]-2H-1,4-benzoxazin-3(4H)-one
7-[4-(trifluoromethyl)phenyl]-1,3,4,5-tetrahydro-2H-1-benzazepin-2-one
8-fluoro-6-[4-(trifluoromethyl)phenyl]-3,4-dihydro-2(1H)-quinolinone
6-(2,2-Difluoro-benzo[1,3]dioxol-5-yl)-3,4-dihydro-1H-quinolin-2-one
6-(3-Fluoro-4-(trifluoromethyl)phenyl)-3,4-dihydro-1H-quinolin-2-one
8-[4-(trifluoromethyl)phenyl]-2,3-dihydro-1,5-benzothiazepin-4(5H)-one
7-(4-Trifluoromethanesulfonyl-phenyl)-4H-benzo[1,4]thiazin-3-one
7-(3-Fluoro-4(trifluoromethyl)phenyl)-4H-benzo[1,4]thiazin-3-one
8-fluoro-6-[3-fluoro-4-(trifluoromethyl)phenyl]-3,4-dihydro-2(1H)-quinolinone
6-[3-nitro-4-(trifluoromethyl)phenyl]-3,4-dihydro-2(1H)-quinolinone
6-[3-chloro-4-(trifluoromethyl)phenyl]-3,4-dihydro-2(1H)-quinolinone
8-amino-6-[4-(trifluoromethyl)phenyl]-3,4-dihydro-2(1H)-quinolinone
7-(4-Trifluoromethanesulfonyl-phenyl)-1,3,4,5-tetrahydro-benzo[b]azepin-2-one
7-(3-Fluoro-4(trifluoromethyl)phenyl)-1,3,4,5-tetrahydro-benzo[b]azepin-2-one
7-[4-(trifluoromethyl)phenyl]-1,5-dihydro-4,1-benzothiazepin-2(3H)-one
±3-Hydroxy-6-[4-(trifluoromethyl)phenyl]-3,4-dihydro-2(1H)-quinolinone
7-[4-(trifluoromethyl)phenyl]-1,5-dihydro-4,1-benzoxazepin-2(3H)-one
6-[3,5-difluoro-4-(trifluoromethyl)phenyl]-3,4-dihydro-2(1H)-quinolinone
7-[4-(Trifluoromethyl)phenyl]-1,3-dihydro-2H-1,4-benzodiazepin-2-one
8-amino-6-[3-fluoro-4-(trifluoromethyl)phenyl]-3,4-dihydro-2(1H)-quinolinone
2-Oxo-6-[4-(trifluoromethyl)phenyl]-1,2,3,4-tetrahydro-3-quinolinecarboxylic acid
(+)-3-Hydroxy-6-[4-(trifluoromethyl)phenyl]-3,4-dihydro-2(1H)-quinolinone
6-[4-bromo-3-(trifluoromethyl)phenyl]-3,4-dihydro-2(1H)-quinolinone
6-[4-(trifluoromethyl)-1-cyclohexen-1-yl]-3,4-dihydro-2(1H)-quinolinone
±6-[3,5-difluoro-4-(trifluoromethyl)phenyl]-3-hydroxy-3,4-dihydro-2(1H)-quinolinone
±6-[3-fluoro-4-(trifluoromethyl)phenyl]-3-hydroxy-3,4-dihydro-2(1H)-quinolinone;
and pharmaceutically acceptable derivatives and solvates thereof.

17. A composition comprising a pharmaceutically acceptable excipient and a compound or derivative or solvate thereof of any one of claims 1-16.

18. A method of modulating KSP kinesin activity or of inhibiting KSP which comprises contacting said kinesin with an effective amount of a compound or derivative or solvate thereof, or a composition according to any one of claims 1-16.

19. A method for the treatment of a disease of proliferating cells comprising administering to a subject in need thereof a compound or derivative or solvate thereof, or a composition according to any one of claims 1-16.

20. A method according to claim 19 wherein said disease is selected from the group consisting of cancer, hyperplasias, restenosis, cardiac hypertrophy, immune disorders, fungal disorders and inflammation.
